(19) United States
(12) Patent Application Publication
(10) Pub. No.: US 2014/0186872 A1
(43) Pub. Date: Jul. 3, 2014

(54) BISACODYL AND ITS ANALOGUES AS DRUGS FOR USE IN THE TREATMENT OF CANCER

(71) Applicants: Marie Feve, Illkirch-Graffenstaden (FR); Maria Zeniou-Meyer, Strasbourg (FR); Jacques Haiech, Strasbourg (FR); Herve Chneiweiss, Paris (FR); Marie-Claude Kilhoffer, Strasbourg (FR); Samir Mameri, Saverne (FR); Marcel Hibert, Eschau (FR)

(72) Inventors: Marie Feve, Illkirch-Graffenstaden (FR); Maria Zeniou-Meyer, Strasbourg (FR); Jacques Haiech, Strasbourg (FR); Herve Chneiweiss, Paris (FR); Marie-Claude Kilhoffer, Strasbourg (FR); Samir Mameri, Saverne (FR); Marcel Hibert, Eschau (FR)

(73) Assignees: CENTRE NATIONAL DE LA RECHERCHE SCIENTIFIQUE- CNRS, Paris Cedex 16 (FR); UNIVERSITE DE STRASBOURG, Strasbourg (FR)

(21) Appl. No.: 14/124,303

(22) PCT Filed: Jun. 6, 2012
(86) PCT No.: PCT/IB2012/052861

§ 371 Date: Mar. 10, 2014

Related U.S. Application Data
(60) Provisional application No. 61/614,680, filed on Mar. 23, 2012.

(30) Foreign Application Priority Data
Jun. 6, 2011 (FR) 1154910

(51) Int. Cl. A61K 31/4402; C07D 213/30; C07D 213/34; C07D 213/16; C07D 213/26; G01N 33/50; C07F 7/18

(52) U.S. Cl. CPC A61K 31/4402; G01N 33/5011; C07D 213/30; C07F 7/1804; C07D 213/16; C07D 213/26; C07D 213/34
USPC 435/29; 546/342; 546/343; 546/14; 546/350; 546/346

(57) ABSTRACT

The present invention provides compounds having the formula A: (A) or pharmaceutically acceptable salt thereof, wherein W, R1, R2 and R5 are as defined in classes and subclasses herein, and pharmaceutical compositions thereof, as described generally and in subclasses herein, which compounds are useful as cytotoxic agents towards proliferating and/or quiescent cancer stem cells, and thus are useful, for example, for the treatment of cancer.

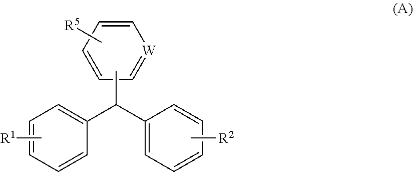

### PRIORITY

The present patent application claims priority to French Patent Application No FR 11/54910 filed on Jun. 6, 2011 and U.S. Provisional Application No. 61/614,680 filed on 23 Mar. 2012, the entire contents of each of which are hereby incorporated herein by reference.

### DESCRIPTION

1. Technical Field

The present invention relates to the field of the prevention and treatment of diseases involving abnormal cellular proliferation and/or loss of cell differentiation.

It relates more precisely to bisacodyl and its analogues as medicinal products intended for treating cancer. It also relates to pharmaceutical compositions comprising bisacodyl or an analogue thereof as medicinal products intended for treating cancer. These pharmaceutical compositions can notably be intended for preventing or treating diseases involving abnormal cellular proliferation, notably cancer.

The present invention notably finds application in treatments for cancer involving cancer stem cells, in particular quiescent.

In the following description, the references between square brackets [ ] refer to the list of references given at the end of the text.

2. Background

Cancer is a major cause of mortality and consequently is one of the most serious public health problems in the world today. In France, cancer is responsible for about 30% of deaths.

Today, a third of new cancer cases display multiple drug resistance (MDR) or are resistant to drug treatment. This resistance is a major problem from the therapeutic standpoint, but also from the psychological standpoint for the patients.

Malignant tumours are heterogeneous tissues consisting of cells that are more or less differentiated and cancer stem cells (CSCs), having properties of self-renewal and differentiation and considered to be the cells responsible for tumour development. The CSCs are particularly resistant to chemotherapy and radiotherapy and therefore appear to be involved in tumour recurrence after treatment by conventional radiotherapy or chemotherapies.

Thus, it is clear that the development of effective treatments involves targeting not only the cells constituting the tumour mass, but also the CSCs. These CSCs can oscillate between a proliferative state and a quiescent state, the equilibrium between the two states depending on the type of cancer and its environment.

These cancer stem cells have been identified in several types of tumours, notably in: gliomas and in particular glioblastomas as described in the documents: Patru, C., Romao, L., Varlet, P., Coulombel, L., Raponi, E., Cadusseau, J., Renault-Mihara, F., Thirant, C., Leonard, N., Berhneim, A., Mihalescu-Maingot, M., Haiech, J., Bieche, I., Moura-Neto, V., Daumas Duport, C., Junier, M. P., and Chneiweiss, H. “CD133, CD15/SSEA-1, C037 or side populations do not resume tumor-initiating properties of long-term cultured cancer stem cells from human malignant glio-neuronal tumors.” BMC Cancer 10, 66 [1] and Singh, S. K., Clarke, I. D., Terasaki, M., Bonn, V. E., Hawkins, C., Squire, J., and Dirks, P. B. (2003). Identification of a cancer stem cell in human brain tumors. Cancer Res 63, 5821-5828 [2], Thirant C, Bessette B, Varlet P, Puget S, Cadusseau J, Dos Reis Tavares S, Studler J M, Silvestre D C, Susini A, Villa C, Miguel C, Bogeas A, Surena A L, Dias-Morais A, Leonard N, Pflumio F, Bièche I, Boussin F D, Sainte-Rose C, Grill J, Daumas-Duport C, Chneiweiss H, Junier M P. Clinical relevance of tumor cells with stem-like properties in pediatric brain tumors. PLoS One. 2011 Jan. 28; 6(1):e16375 [3]; Galan-Moya E M, Le Guelte A, Lima-Fernandes E, Thirant C, Dwyer J, Bidere N, Couraud P O, Scott M, Junier M P, Chneiweiss H, Gavard J. Brain endothelial cells maintain glioblastoma stem-like cell expansion through the mTOR pathway. EMBO Report 2011, 12, 479-476; [4]; Silvestre D C, Pineda Marti J R, Hoffschir F, Studler J M, Mouthon M A, Pflumio F, Junier M P, Chneiweiss H, Boussin F D. Alternative Lengthening of Telomeres in Human Glioma Stem Cells. Stem Cells. 2011 Jan. 14 [5]; melanomas as described in the document: Schatton, T., Murphy, G. F., Frank, N. Y., Yamaura, K., Waaga-Gasser, A. M., Gasser, M., Zhan, Q., Jordan, S., Duncan, L. M., Weishaupt, C., Fuhlbrigge, R. C., Kupper, T. S., Sayegh, M. H., and Frank, M. H. (2008). “Identification of cells initiating human melanomas”. Nature 451, 345-349 [6], tumours of the haemato-lymphoid system as described in the documents: Reya, T., Morrison, S. J., Clarke, M. F., and Weissman, I. L. (2001) “Stem cells, cancer, and cancer stem cells.” Nature 414, 105-111 [7] and Rosen, J. M., and Jordan, C. T. (2009) “The increasing complexity of the cancer stem cell paradigm” Science 324, 1670-1673 [8], mammary tumours [8].

Owing to their properties of self-renewal and differentiation, these cancer stem cells initiate and guide the formation and growth of tumours [7][8].

The resistance of cancer stem cells to radiotherapy and to chemotherapy has been demonstrated. It has in fact been reported that these cells are most often resistant to existing therapies [8] and notably to temozolomide (TMZ) in the case of glioblastomas [1].

TMZ is unable to eradicate tumours, since reappearance or aggravation of tumours can be observed after stopping treatment, on average 2.9 months for a glioblastoma multiforme and 5.4 months for an anaplastic astrocytoma as described in the document: European Medicines Agency (2009) “European Public Assessment Report (EPAR) Temodal”—EPAR summary for the public [9].

Moreover, the use of temozolomide is associated with several undesirable effects, those observed most frequently being: nausea, vomiting, constipation, anorexia, alopecia, headaches, fatigue, convulsions, skin rash, neutropenia, lymphopenia, thrombocytopenia. This applies to most of the anticancer drugs used at present. Certain compounds used in cancer treatment, for example vinblastine, can also induce the development of drug resistant tumour cells.

Moreover, the majority of anticancer treatments target cells in active proliferation whereas one of the major properties of CSCs is their capacity for prolonged dormancy (or quiescence).

There is therefore a need for the development of compounds having improved properties for treating cancers. In particular, there is a real need to develop new anticancer compounds that are less toxic and have a minimum of side-effects, specifically targeting a type of cellular population to attack the cancer at its source and eliminate all of the cancerous cells. In particular, there is a need to develop anticancer compounds capable of affecting the viability of quiescent cancer stem cells. Specifically, there is a great need to develop compounds targeting both proliferating and quiescent tumor stem cells, for the treatment of cancer.

### DESCRIPTION

Because of their location, invasiveness and relative resistance to standard therapies, treating malignant brain tumors is challenging. This is especially true for glioblastoma, the most common and advanced grade of astrocytic tumors (1). Current glioblastoma treatments combine surgery to radiotherapy and chemotherapy with temozolomide (TMZ), a DNA alkylating agent (2). Despite this multiple therapeutic approach, median survival of glioblastoma patients rarely exceeds 2 years (3).

Glioblastomas are histopathologically heterogeneous with cells characterized by various degrees of proliferative ability, differentiation and/or invasiveness (4). In recent years, the cancer stem cell model was proposed to explain tumor heterogeneity (5) (6). Indeed, a subpopulation of malignant cancer stem cells with tumor-propagating capacity and self-renewal as well as differentiation ability to give rise to bulk populations of non tumorigenic cancer cells, was evidenced and characterized in hematopoietic malignancies (7, 8) and in solid tumors including brain (9-13), breast (14) and colon cancer (15-17) as well as melanoma (18, 19).

Cancer stem cells were also proposed to participate to tumor recurrences after treatment (20). Indeed, glioblastoma stem cells are more resistant to radiation-induced apoptosis through more efficient DNA repair responses (21) and were shown to be chemo-resistant through increased expression of drug transporters (22, 23). Finally, impaired functioning of apoptotic pathways was described in glioma stem-like cells (24).

The relative radio- and chemo-resistance of cancer stem cells, as well as their ability to favor angiogenesis and thus, tumor growth (25), led to a new paradigm in cancer therapy postulating that efficient cancer treatment should also target cancer stem cells either by killing them or by forcing them to acquire a more differentiated state which is more sensitive to conventional treatments (20). As a consequence, new strategies targeting cancer stem cells were developed. These include specific signaling pathway inhibition through, for example, the use of γ-secretase inhibitors to affect Notch signaling which has been extensively involved in cancer stem cell self-renewal and fate determination (26), or Akt inhibitors to affect EGFR (epidermal growth factor receptor)-mediated growth signaling through phosphoinositide 3-kinase (PI3K) which is critical to cancer stem cell physiopathology (27) (28). Alternatively, bone morphogenic proteins (BMPs) were used to induce cancer stem cell differentiation (29). Radio-resistance of cancer stem cells was reduced both by inhibitors of checkpoint kinases 1 and 2, participating in DNA repair processes (21) and through inhibition of the Notch signaling pathway which was also involved in this phenomenon (30). Moreover, antiangiogenic strategies were used to destroy the vascular niche of cancer stem cells, thus leading to their elimination (31-33) and miRNAs were shown to affect self-survival and infiltration properties of glioma stem cells thus inhibiting tumor development in vivo (34). Finally, chemical screens have led to the identification of specific inhibitors of breast cancer stem cells (35) and glioma stem-cell enriched cultures (36, 37).

It is noteworthy that all of the above studies concern the targeting of proliferating tumor stem cells despite increasing evidence arguing in favor of the existence of relatively quiescent cancer stem cells within the tumor bulk in vivo (38). Indeed, slowly proliferating cells with stem cell properties and tumor-initiation ability were identified in several solid tumors including ovarian, liver and breast cancer as well as in melanoma (39-41) (42). In addition, a slow-cycling stem cell subpopulation from pancreatic adenocarcinoma was shown to have increased tumorigenic and invasive potential compared to faster-cycling cells from these tumors (43).

More importantly, the quiescent state was proposed to contribute to the resistance of cancer stem cells to current chemotherapeutic agents. It was shown, for instance, that cancer stem cells of acute and chronic myeloid leukemia survive in the dormant G0 phase of the cell cycle after chemotherapy and that relapses and metastases of breast cancer often occur after long intervals suggesting the involvement of cancer stem cells in a deep dormant phase (44-46). Finally, several studies have reported the resistance of relatively quiescent cells from ovarian, breast and pancreatic tumors to conventional treatments (39, 43, 47).

Surprisingly, the inventors discovered that bisacodyl and its analogues have precisely this novel and major property of affecting the viability of quiescent cancer stem cells, and therefore of completely or partly solving the problems mentioned above.

### 1/Compounds

The present invention has precisely the aim of meeting this need by providing compounds having the following formula A:

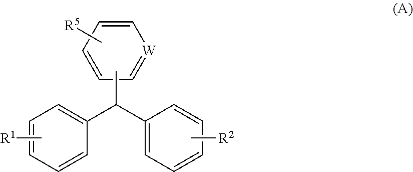

or a pharmaceutically acceptable salt thereof, wherein:
R¹ and R² represent independently —H; —OH; F; Cl; Br; I; —NR^{a}R^{b} where R^{a} and R^{b} represent independently H or a linear, branched or cyclic C₁₋₆ alkyl group and where R^{a} and R^{b} can form, together with the nitrogen atom to which they are attached, a heterocycle with 5 or 6 ring members; —OR; —C(O)—NH—R; —O—C(O)—R; —NH—C(O)—R; —NH—SO₂—R; —OSiR^{c}₃ where each occurrence of R^{c} represents, independently of the other occurrences of R^{c}, a linear, branched or cyclic C₁₋₆ alkyl group, —OSO₃⁻; —OPO₃²⁻; —OSO₃H; —OPO₃H₂; where each occurrence of R represents, independently of the other occurrences of R, a hydrogen atom or an optionally substituted linear, branched or cyclic C₁₋₆ alkyl, C₂₋₆ alkene, C₂₋₆ alkyne or C₁₋₆ haloalkyl group; and where at least one of the radicals R¹ and R² is different from H;
W represents C(—R³), N or N⁺(—R⁴) in which R³ represents H; —OH; F; Cl; Br; I; —NR^{a}R^{b} where R^{a} and R^{b} represent independently H or a linear, branched or cyclic C₁₋₆ alkyl group and where R^{a} and R^{b} can form, together with the nitrogen atom to which they are attached, a heterocycle with 5 or 6 ring members; —OR where R represents an optionally substituted linear, branched or cyclic C₁₋₆ alkyl, C₂₋₆ alkene, C₂₋₆ alkyne or C₁₋₆ haloalkyl group; or —C(O)OR^{d} where R^{d} represents H or a linear, branched or cyclic C₁₋₆ alkyl group; and R⁴ represents a linear, branched or cyclic C₁₋₆ alkyl group;
R⁵ represents a hydrogen atom; a linear or branched C₁₋₆ alkyl group; —OH; F; Cl; Br; I; —CF₃; —NO₂; —OR′ wherein R′ represents a hydrogen atom or an optionally substituted linear, branched or cyclic C₁₋₆ alkyl or C₁₋₆ haloalkyl group; or —NR^{c}R^{d} wherein R^{c} et R^{d} independently represent H, a linear, branched or cyclic C₁₋₆ alkyl group, R^{a} et R^{b} and where R^{c} and R^{d} can form, together with the nitrogen atom to which they are attached, a heterocycle with 5 or 6 ring members;
   for use as a medicinal product intended for treating cancer.

Advantageously, the medicinal product is intended for treating cancers containing cancer stem cells and tumour initiating cells.

For example, compounds of the invention may have the following structure:

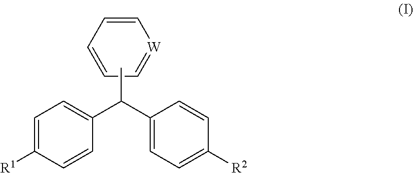

or a pharmaceutically acceptable salt thereof, in which W, R¹ and R² are as defined above.

In the compounds defined above, R¹ and R² may also independently represent an optionally substituted linear, branched or cyclic C₁₋₆alkyl, C₂₋₆alkenyl, C₂₋₆alkynyl or C₁₋₆haloalkyl radical. Advantageously, R¹ and R² may independently represent a linear, branched of cyclic C₁₋₆alkyl or C₁₋₆haloalkyl moiety.

The group W can be in ortho, meta, or para position relative to the point of linkage of the aromatic ring to the rest of the molecule.

For example, the group W can be in the ortho position, and the compound can have the following formula I^{A}:

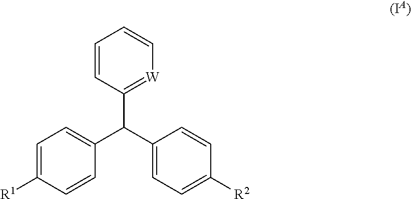

or a pharmaceutically acceptable salt thereof, in which the groups W, R¹ and R² are as defined above.

The group W can represent N, and the compound can have the following formula I^{B}:

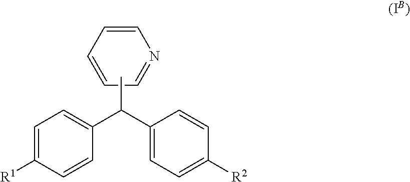

or a pharmaceutically acceptable salt thereof, in which the groups R¹ and R² are as defined above. The nitrogen atom can be in the ortho position, and the compound can have the following formula I^{C}:

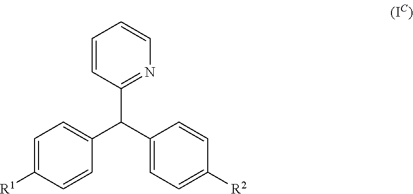

or a pharmaceutically acceptable salt thereof, in which the groups R¹ and R² are as defined above. Advantageously, at least one of R¹ or R² represents —OH; an optionally substituted linear, branched or cyclic C₁₋₆alkyl, C₂₋₆alkenyl, C₂₋₆alkynyl or C₁₋₆haloalkyl radical; —OR; —O—C(O)—R; —OSiR^{c}₃ wherein each occurrence of R^{c} independently represents a linear, branched or cyclic C₁₋₆alkyl radical; —OSO₃⁻; —OPO₃²⁻; —OSO₃H; —OPO₃H₂; —OPO₃R₂; wherein each occurrence of R independently represents a hydrogen atom, or an optionally substituted linear, branched or cyclic C₁₋₆alkyl, C₂₋₆alkenyl, C₂₋₆alkynyl or C₁₋₆haloalkyl radical; with the proviso that R¹ and R² may not be independently hydrogen, methyl, C1-C3 alkoxy, halogen, nitro, or trifluoromethyl.

The group W can represent C(—R³), and the compound can have the following formula I^{D}:

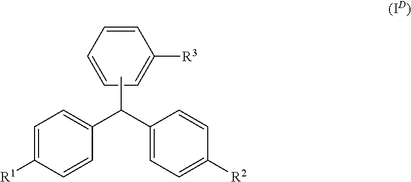

or a pharmaceutically acceptable salt thereof, in which the groups R¹, R² and R³ are as defined above. Group R³ can be in the ortho position, and the compound can have the following formula I^{E}:

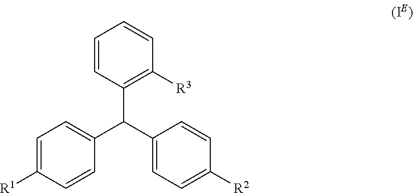

or a pharmaceutically acceptable salt thereof, in which the groups R¹, R² and R³ are as defined above.

The group W can represent N⁺(—R⁴), and the compound can have the following formula I^{F}:

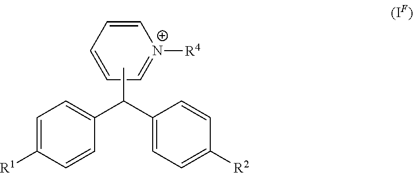

or a pharmaceutically acceptable salt thereof, in which the groups R¹, R² and R⁴ are as defined above. The group N⁺(—R⁴) can be in the ortho position, and the compound can have the following formula I^{G}:

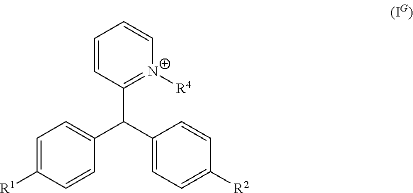

or a pharmaceutically acceptable salt thereof, in which the groups R¹, R² and R⁴ are as defined above. For example, the radical R⁴ can represent a methyl, an ethyl or a propyl. Advantageously, R⁴ can represent the methyl radical.

Advantageously, in each of the formulae I, I^{A} to I^{G} and each of the embodiments relating to these, R¹ and R² can represent independently —H; —OH; F; Cl; Br; I; —NR^{a}R^{b} where R^{a} and R^{b} represent independently H or a methyl, ethyl, n-propyl, iso-propyl, n-butyl, isobutyl, tert-butyl, n-pentyl, sec-pentyl, n-hexyl or sec-hexyl group and where R^{a} and R^{b} can form, together with the nitrogen atom to which they are attached, a pyrrolidinyl or piperidinyl group; —OR; —C(O)—NH—R; —O—C(O)—R; —NH—C(O)—R; —NH—SO₂—R; —OSiR^{c}₃ where each occurrence of R^{c} represents, independently of the other occurrences of R^{c}, a methyl, ethyl, n-propyl, iso-propyl, isobutyl or tert-butyl group; —OSO₃⁻; —OPO₃²⁻; —OSO₃H; —OPO₃H₂; where each occurrence of R represents, independently of the other occurrences of R, a hydrogen atom or a methyl, ethyl, propyl, isopropyl, isobutyl, tert-butyl, halomethyl, haloethyl, ethylenyl, allyl, or propynyl group; and where at least one of the radicals R¹ and R² is different from H. Advantageously, in each of the formulae I, I^{A} to I^{G} and each of the embodiments relating to these, R¹ and R² can also represent independently an optionally substituted linear, branched or cyclic C₁₋₆alkyl, C₂₋₆alkenyl, C₂₋₆alkynyl or C₁₋₆haloalkyl radical;

Advantageously, in each of the formulae I, I^{A} to I^{G} and each of the embodiments relating to these, R¹ or R² can represent independently —H; —OH; F; Cl; Br; I; —NH(R^{a}) where R^{a} represents a hydrogen atom or a methyl, ethyl, n-propyl, iso-propyl, n-butyl, isobutyl, tert-butyl, n-pentyl, sec-pentyl, n-hexyl or sec-hexyl group; —OR; —C(O)—NH—R; —O—C(O)—R; —NH—C(O)—R; —NH—SO₂—R; —OSiR^{c}₃ where each occurrence of R^{c} represents, independently of the other occurrences of R^{c}, a methyl, ethyl, n-propyl, iso-propyl, isobutyl or tert-butyl group; —OSO₃⁻; —OPO₃²⁻; —OSO₃H; —OPO₃H₂; where each occurrence of R represents, independently of the other occurrences of R, a hydrogen atom or a methyl, ethyl, propyl, isopropyl, isobutyl, tert-butyl, halomethyl, haloethyl, ethylenyl, allyl, or propynyl group; and where at least one of the radicals R¹ and R² is different from H.

Advantageously, in each of the formulae I, I^{A} to I^{G} and each of the embodiments relating to these, at least one of the radicals R¹ or R² can represent —H; —OH; —NH(R^{a}) where R^{a} represents a hydrogen atom or a methyl, ethyl, n-propyl, iso-propyl, n-butyl, isobutyl, tert-butyl, n-pentyl, sec-pentyl, n-hexyl or sec-hexyl group; —OR; —O—C(O)—R; —NH—C(O)—R; —NH—SO₂—R; —OSiR^{c}₃ where each occurrence of R^{c} represents, independently of the other occurrences of R^{c}, a methyl, ethyl, n-propyl, iso-propyl, isobutyl or tert-butyl group; —OSO₃⁻; —OPO₃²⁻; —OSO₃H; —OPO₃H₂; where each occurrence of R represents, independently of the other occurrences of R, a hydrogen atom or a methyl, ethyl, propyl, isopropyl, isobutyl, tert-butyl, halomethyl, haloethyl, ethylenyl, allyl, or propynyl group; and where at least one of the radicals R¹ and R² is different from H. Advantageously, in the compounds of formulae I, I^{A} to I^{G} and each of the embodiments relating to these, each occurrence of R may represent, independently of the other occurrences of R, a methyl, ethyl, propyl, isopropyl, isobutyl, tert-butyl, halomethyl, haloethyl, ethylenyl, allyl, or propynyl group.

Advantageously, in each of the formulae I, I^{A} to I^{G} and each of the embodiments relating to these, R¹ or R² can represent independently —H; —OH; F; Cl; Br; I; —NH₂; —NH(R^{a}) where R^{a} represents a methyl, ethyl, n-propyl, iso-propyl, n-butyl, isobutyl, tert-butyl, n-pentyl, sec-pentyl, n-hexyl or sec-hexyl group; —OCH₃; —O—CH₂—C≡CH, —O—CH₂—CH₂—CH₃, —C(CH₃)—CH₂—CH₃—CH₃; —O—C(O)—CH₃; —O—C(O)—CF₃; or —O—Si(CH₃)₂—C(CH₃)₃; and where at least one of the radicals R¹ and R² is different from H.

Advantageously, in each of the formulae I, I^{A} to I^{G} and each of the embodiments relating to these, R¹ or R² can represent independently —H; —OH; —NH(R^{a}); —OCH₃; —O—CH₂—C≡CH, —O—CH₂—CH₂—CH₃, —C(CH₃)—CH₂—CH₃—CH₃; —O—C(O)—CH₃; —O—C(O)—CF₃; or —O—Si(CH₃)₂—C(CH₃)₃; where R^{a} represents a methyl, ethyl, n-propyl, or iso-propyl group;
and where at least one of the radicals R¹ and R² is different from H.

In one embodiment, in each of the formulae I, I^{A} to I^{G} and each of the embodiments relating to these, R¹ and R² are different from —H, and can represent independently:
(i) —OH; F; Cl; Br; I; —NR^{a}R^{b} where R^{a} and R^{b} represent independently H or a methyl, ethyl, n-propyl, iso-propyl, n-butyl, isobutyl, tert-butyl, n-pentyl, sec-pentyl, n-hexyl or sec-hexyl group, and where R^{a} and R^{b} can form, together with the nitrogen atom to which they are attached, a pyrrolidinyl or piperidinyl group; —OR; —C(O)—NH—R; —O—C(O)—R; —NH—C(O)—R; —NH—SO₂—R; —OSiR^{c}₃ where each occurrence of R^{c} represents, independently of the other occurrences of R^{c}, a methyl, ethyl, n-propyl, iso-propyl, isobutyl or tert-butyl group; —OSO₃⁻; —OPO₃²⁻; —OSO₃H; —OPO₃H₂; where each occurrence of R represents, independently of the other occurrences of R, a hydrogen atom or a methyl, ethyl, propyl, isopropyl, isobutyl, tert-butyl, halomethyl, haloethyl, ethylenyl, allyl, or propynyl group; Advantageously, each occurrence of R may represent, independently of the other occurrences of R, a methyl, ethyl, propyl, isopropyl, isobutyl, tert-butyl, halomethyl, haloethyl, ethylenyl, allyl, or propynyl group;
(ii) —OH; F; Cl; Br; I; —NH(R^{a}) where R^{a} represents a hydrogen atom or a methyl, ethyl, n-propyl, iso-propyl, n-butyl, isobutyl, tert-butyl, n-pentyl, sec-pentyl, n-hexyl or sec-hexyl group; —OR; —C(O)—NH—R; —O—C(O)—R; —NH—C(O)—R; —NH—SO₂—R; —OSiR^{c}₃ where each occurrence of R^{c} represents, independently of the other occurrences of R^{c}, a methyl, ethyl, n-propyl, iso-propyl, isobutyl or tert-butyl group; —OSO₃⁻; —OPO₃²⁻; —OSO₃H; —OPO₃H₂; where each occurrence of R represents, independently of the other occurrences of R, a hydrogen atom or a methyl, ethyl, propyl, isopropyl, isobutyl, tert-butyl, halomethyl, haloethyl, ethylenyl, allyl, or propynyl group; Advantageously, each occurrence of R may represent, independently of the other occurrences of R, a methyl, ethyl, propyl, isopropyl, isobutyl, tert-butyl, halomethyl, haloethyl, ethylenyl, allyl, or propynyl group;
(iii) —OH; —NH(R^{a}) where R^{a} represents a hydrogen atom or a methyl, ethyl, n-propyl, iso-propyl, n-butyl, isobutyl, tert-butyl, n-pentyl, sec-pentyl, n-hexyl or sec-hexyl group; —OR; —O—C(O)—R; —NH—C(O)—R; —NH—SO₂—R; —OSiR^{c}₃ where each occurrence of R^{c} represents, independently of the other occurrences of R^{c}, a methyl, ethyl, n-propyl, iso-propyl, isobutyl or tert-butyl group; —OSO₃⁻; —OPO₃²⁻; —OSO₃H; —OPO₃H₂; where each occurrence of R represents, independently of the other occurrences of R, a hydrogen atom or a methyl, ethyl, propyl, isopropyl, isobutyl, tert-butyl, halomethyl, haloethyl, ethylenyl, allyl, or propynyl group; Advantageously, each occurrence of R may represent, independently of the other occurrences of R, a methyl, ethyl, propyl, isopropyl, isobutyl, tert-butyl, halomethyl, haloethyl, ethylenyl, allyl, or propynyl group;
(iv) —OH; F; Cl; Br; I; —NH₂; —NH(R^{a}) where R^{a} represents a methyl, ethyl, n-propyl, iso-propyl, n-butyl, isobutyl, tert-butyl, n-pentyl, sec-pentyl, n-hexyl or sec-hexyl group; —OCH₃; —O—CH₂—C≡CH, —O—CH₂—CH₂—CH₃, —C(CH₃)—CH₂—CH₃—CH₃; —O—C(O)—CH₃; —O—C(O)—CF₃; or —O—Si(CH₃)₂—C(CH₃)₃; or
(v) —OH; —NH(R^{a}); —OCH₃; —O—CH₂—C≡CH, —O—CH₂—CH₂—CH₃, —C(CH₃)—CH₂—CH₃—CH₃; —O—C(O)—CH₃; —O—C(O)—CF₃; or —O—Si(CH₃)₂—C(CH₃)₃; where R^{a} represents a methyl, ethyl, n-propyl, or iso-propyl group.

Advantageously, in each of the formulae I and I^{A} and each of the embodiments relating to these, W can represent C(—R³) in which R³ represents H; —OH; F; Cl; Br; I; —NR^{a}R^{b} where R^{a} and R^{b} represent independently H or a methyl, ethyl, n-propyl, iso-propyl, n-butyl, isobutyl, tert-butyl, n-pentyl, sec-pentyl, n-hexyl or sec-hexyl group and where R^{a} and R^{b} can form, together with the nitrogen atom to which they are attached, a pyrrolidinyl or piperidinyl group; —OR where R represents a hydrogen atom or a methyl, ethyl, propyl, isopropyl, isobutyl, tert-butyl, halomethyl, haloethyl, ethylenyl, allyl, or propynyl group; or —C(O)OR^{d} where R^{d} represents a hydrogen atom or a methyl, ethyl, propyl, isopropyl, isobutyl or tert-butyl group.

Advantageously, in each of the formulae I and I^{A} and each of the embodiments relating to these, W can represent C(—R³) in which R³ represents H; —OH; F; Cl; Br; I; —NH₂; —OCH₃; —C(O)OH or —C(O)OCH₃.

Advantageously, in each of the formulae I and I^{A} and each of the embodiments relating to these, W can represent CH.

Advantageously, the compounds can correspond to the following formula I^{H}.

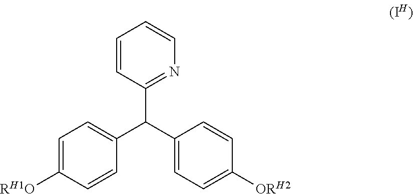

or a pharmaceutically acceptable salt thereof, in which R^{H1} and R^{H2} represent independently —H; —R; —C(O)—R; —SiR^{c}₃ where each occurrence of R^{c} represents, independently of the other occurrences of R^{c}, a linear, branched or cyclic C₁₋₆ alkyl group; —SO₃; —PO₃²⁻; —SO₃H; —PO₃H₂; where each occurrence of R represents, independently of the other occurrences of R, a hydrogen atom or an optionally substituted linear, branched or cyclic C₁₋₆ alkyl, C₂₋₆ alkene, C₂₋₆ alkyne or C₁₋₆ haloalkyl group. Advantageously, each occurrence of R may represent, independently of the other occurrences of R, an optionally substituted linear, branched or cyclic C₁₋₆ alkyl, C₂₋₆ alkene, C₂₋₆ alkyne or C₁₋₆ haloalkyl group.

Advantageously, in the compounds of formula I^{H} and each of the embodiments relating to these, R^{H1} and R^{H2} can represent independently —H; —R; —C(O)—R; —SiR^{c}₃ where each occurrence of R^{c} represents, independently of the other occurrences of R^{c}, a methyl, ethyl, n-propyl, iso-propyl, isobutyl or tert-butyl group; —SO₃⁻; —PO₃²⁻; —SO₃H; —PO₃H₂; where each occurrence of R represents, independently of the other occurrences of R, a hydrogen atom or a methyl, ethyl, propyl, isopropyl, isobutyl, tert-butyl, halomethyl, haloethyl, ethylenyl, allyl, or propynyl group. Advantageously, each occurrence of R may represent, independently of the other occurrences of R, a methyl, ethyl, propyl, isopropyl, isobutyl, tert-butyl, halomethyl, haloethyl, ethylenyl, allyl, or propynyl group.

Advantageously, in the compounds of formula I^{H} and each of the embodiments relating to these, R^{H1} and R^{H2} can represent independently —H; —R; —C(O)—R; —SiR^{c}₃ where each occurrence of R^{c} represents, independently of the other occurrences of R^{c} a methyl, ethyl, n-propyl, iso-propyl, isobutyl or tert-butyl group; —SO₃⁻; —PO₃²⁻; —SO₃H; —PO₃H₂; where each occurrence of R represents, independently of the other occurrences of R, a hydrogen atom or a methyl, ethyl, propyl, isopropyl, isobutyl, tert-butyl, halomethyl, haloethyl, ethylenyl, allyl, or propynyl group. Advantageously, in the compounds of formula I^{H} and each of the embodiments relating to these, each occurrence of R represents, independently of the other occurrences of R, a methyl, ethyl, propyl, isopropyl, isobutyl, tert-butyl, halomethyl, haloethyl, ethylenyl, allyl, or propynyl group.

Advantageously, in the compounds of formula I^{H} and each of the embodiments relating to these, R^{H1} and R^{H2} can represent independently —H; —CH₃; —CH₂—C≡CH, —CH₂—CH₂—CH₃, —C(O)—CH₃; —C(O)—CF₃; or —Si(CH₃)₂—C(CH₃)₃.

Advantageously, in the compounds of formula I^{H} and each of the embodiments relating to these, at least one of the radicals R^{H1} or R^{H2} can represent —H; —CH₃; —CH₂—C≡CH, —CH₂—CH₂—CH₃, —C(O)—CH₃; —C(O)—CF₃; or —Si(CH₃)₂—C(CH₃)₃.

Advantageously, the compounds can correspond to the following formula I^{J}.

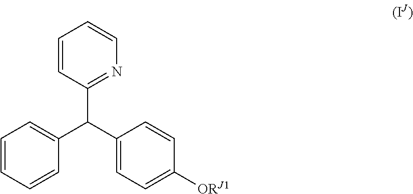

or a pharmaceutically acceptable salt thereof, in which R^{J1} can represent —H; —R; —C(O)—R; —SiR^{c}₃ where each occurrence of R^{c} represents, independently of the other occurrences of R^{c}, a linear, branched or cyclic C₁₋₆ alkyl group; —SO₃; —PO₃²⁻; —SO₃H;
—PO₃H₂; where each occurrence of R represents, independently of the other occurrences of R, a hydrogen atom or an optionally substituted linear, branched or cyclic C₁₋₆ alkyl, C₂₋₆ alkene, C₂₋₆ alkyne or C₁₋₆ haloalkyl group. Advantageously, in the compounds of formula I^{J} and each of the embodiments relating to these, each occurrence of R may represent, independently of the other occurrences of R, an optionally substituted linear, branched or cyclic C₁₋₆ alkyl, C₂₋₆ alkene, C₂₋₆ alkyne or C₁₋₆ haloalkyl group.

Advantageously, in the compounds of formula I^{J} and each of the embodiments relating to these, in which R^{J1} can represent —H; —R; —C(O)—R; —SiR^{c}₃ where each occurrence of R^{c} represents, independently of the other occurrences of R^{c}, a methyl, ethyl, n-propyl, iso-propyl, isobutyl or tert-butyl group; —SO₃; —PO₃²⁻; —SO₃H; —PO₃H₂; where each occurrence of R represents, independently of the other occurrences of R, a hydrogen atom or a methyl, ethyl, propyl, isopropyl, isobutyl, tert-butyl, halomethyl, haloethyl, ethylenyl, allyl, or propynyl group. Advantageously, in the compounds of formula I^{J} and each of the embodiments relating to these, each occurrence of R may represent, independently of the other occurrences of R, a methyl, ethyl, propyl, isopropyl, isobutyl, tert-butyl, halomethyl, haloethyl, ethylenyl, allyl, or propynyl group.

Advantageously, in the compounds of formula I^{J} and each of the embodiments relating to these, in which R^{J1} can represent —H; —R; —C(O)—R; —SiR^{c}₃ where each occurrence of R^{c} represents, independently of the other occurrences of R^{c}, a methyl, ethyl, n-propyl, iso-propyl, isobutyl or tert-butyl group; —SO₃⁻; —PO₃²⁻; —SO₃H;
—PO₃H₂; where each occurrence of R represents, independently of the other occurrences of R, a hydrogen atom or a methyl, ethyl, propyl, isopropyl, isobutyl, tert-butyl, halomethyl, haloethyl, ethylenyl, allyl, or propynyl group. Advantageously, in the compounds of formula I^{J} and each of the embodiments relating to these, each occurrence of R may represent, independently of the other occurrences of R, a methyl, ethyl, propyl, isopropyl, isobutyl, tert-butyl, halomethyl, haloethyl, ethylenyl, allyl, or propynyl group.

Advantageously, in the compounds of formula I^{J} and each of the embodiments relating to these, in which R^{J1} can represent —H; —CH₃; —CH₂—C≡CH, —CH₂—CH₂—CH₃, —C(O)—CH₃; —C(O)—CF₃; or —Si(CH₃)₂—C(CH₃)₃.

Advantageously, the compounds can correspond to the following formula I^{K}.

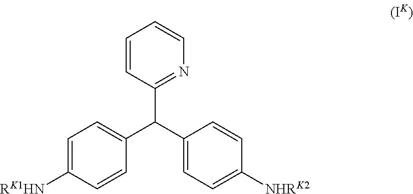

or a pharmaceutically acceptable salt thereof, in which R^{K1} and R^{K2} represent independently —H or a linear, branched or cyclic C₁₋₆ alkyl group.

Advantageously, in the compounds of formula I^{K} and each of the embodiments relating to these, R^{K1} and R^{K2} can represent independently H or a methyl, ethyl, n-propyl, iso-propyl, n-butyl, isobutyl, tert-butyl, n-pentyl, sec-pentyl, n-hexyl or sec-hexyl group.

Advantageously, in the compounds of formula I^{K} and each of the embodiments relating to these, R^{K1} and R^{K2} can represent independently H or a methyl, ethyl, propyl, or isopropyl group.

Advantageously, the compounds can correspond to the following formula I^{L}.

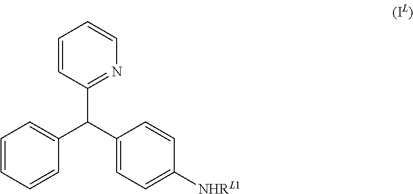

or a pharmaceutically acceptable salt thereof, in which R^{L1} represents —H or a linear, branched or cyclic C₁₋₆ alkyl group.

Advantageously, in the compounds of formula I^{L} and each of the embodiments relating to these, R^{L1} can represent —H or a methyl, ethyl, n-propyl, iso-propyl, n-butyl, isobutyl, tert-butyl, n-pentyl, sec-pentyl, n-hexyl or sec-hexyl group.

Advantageously, in the compounds of formula I^{L} and each of the embodiments relating to these, R^{L1} can represent —H or a methyl, ethyl, propyl, or isopropyl group.

Advantageously, compounds of formula (I) wherein:
W is N; and
R¹ and R² are independently hydrogen, methyl, C1-C3 alkoxy, halogen, nitro, or trifluoromethyl;
   are excluded.

Advantageously, compounds of formula (A) wherein:
W is N;
R¹ and R² are independently hydrogen, methyl, C1-C3 alkoxy, halogen, nitro, or trifluoromethyl; and
R⁵ is hydrogen or methyl;
   are excluded.

Advantageously, compounds of the invention may have the structure:

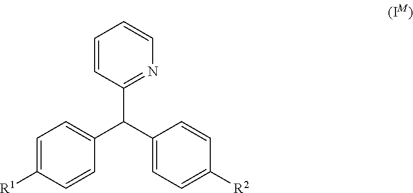

or pharmaceutically acceptable salt thereof;
wherein at least one of R¹ or R² represents —OH; —OR; —O—C(O)—R; —OSiR^{c}₃ wherein each occurrence of R^{c} independently represents a linear, branched or cyclic C₁₋₆alkyl radical; —OSO₃⁻; —OPO₃²⁻; —OSO₃H; —OPO₃H₂; —OPO₃R₂; wherein each occurrence of R independently represents a hydrogen atom, or an optionally substituted linear, branched or cyclic C₁₋₆alkyl, C₂₋₆alkenyl, C₂₋₆alkynyl or C₁₋₆haloalkyl radical. Advantageously, at least one of R¹ or R² may represent OH, or OR wherein R is a hydrolizable group. For example, the hydrolizable group may be a carboxylic ester, a sulphate group, a phosphate group or a —OSiR₃ moiety wherein R is as defined immediately above (independently for each occurrence of R). Advantageously, at least one of R¹ or R² may represent —OH, —O—C(O)—R; —OSiR^{c}₃ wherein each occurrence of R^{c} independently represents a linear, branched or cyclic C₁₋₆alkyl radical; —OSO₃; —OPO₃²; —OSO₃H; —OPO₃H₂; —OPO₃R₂; wherein each occurrence of R independently represents a hydrogen atom, or an optionally substituted linear, branched or cyclic C₁₋₆alkyl, C₂₋₆alkenyl, C₂₋₆alkynyl or C₁₋₆haloalkyl radical. Advantageously, in the compounds of formula I^{M} and each of the embodiments relating to these, each occurrence of R may represent, independently of the other occurrences of R, an optionally substituted linear, branched or cyclic C₁₋₆alkyl, C₂₋₆alkenyl, C₂₋₆alkynyl or C₁₋₆haloalkyl radical.

Advantageously, compounds of the invention may have one of the following structures:

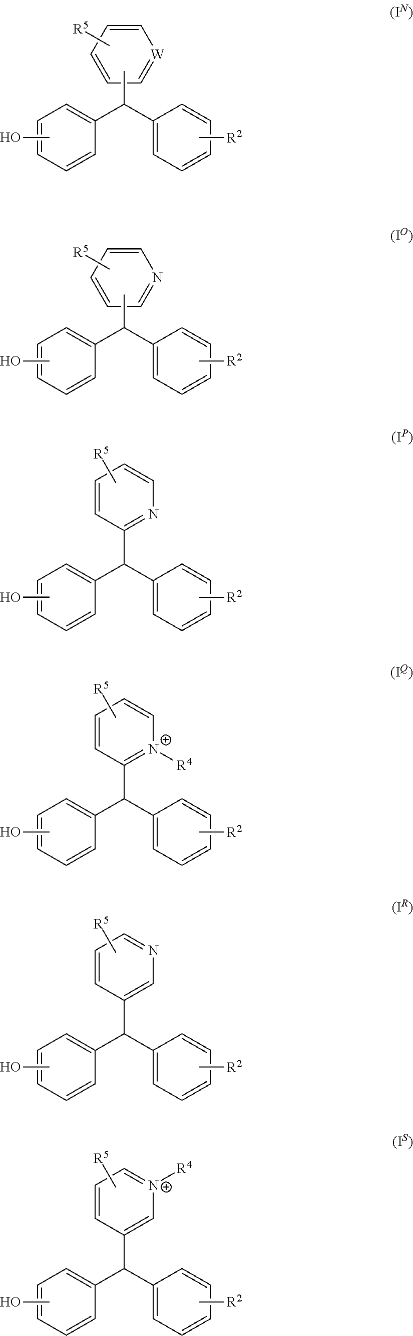

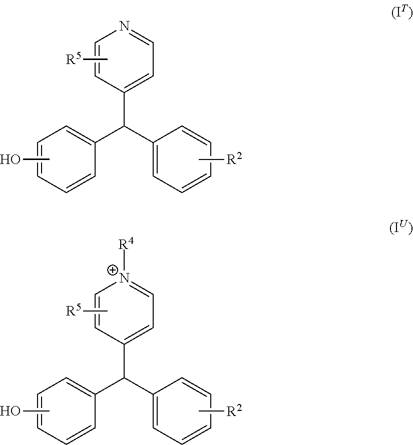

or pharmaceutically acceptable salt thereof;
wherein for each of the structures I^{N} to I^{U}, W, R², R⁴ and R⁵ are as defined above.
Advantageously, for each of the structures I^{N} to I^{U}:
the OH and R² radicals may independently be in ortho, meta or para position on their respective phenyl ring. Advantageously, the OH and R² radicals may each be in para position on their respective phenyl ring; Advantageously, the OH and R² radicals may each be in meta position on their respective phenyl ring; Advantageously, the OH and R² radicals may each be in ortho position on their respective phenyl ring;
R² may represent a halogen atom; a linear or branched C₁₋₆ alkyl group; —N(R)₂; —OH; —O—C₁₋₆haloalkyl; —O—C(O)—R; —OSiR^{c}₃ wherein each occurrence of R^{c} independently represents a linear, branched or cyclic C₁₋₆alkyl radical; —OSO₃⁻; —OPO₃²⁻; —OSO₃H; —OPO₃H₂; —OPO₃R₂; wherein each occurrence of R independently represents a hydrogen atom, or an optionally substituted linear, branched or cyclic C₁₋₆alkyl, C₂₋₆alkenyl, C₂₋₆alkynyl or C₁₋₆haloalkyl radical. Advantageously, R² may represent a halogen atom: —NH₂; —OH, —OCF₃; —O—C(O)—R; —OSO₃; —OPO₃²; —OSO₃H; —OPO₃H₂; —OPO₃R₂; wherein each occurrence of R independently represents a hydrogen atom, or an optionally substituted linear, branched or cyclic C₁₋₆alkyl radical. Advantageously, R² may represent Cl; Br; —NH₂; —OH, —OCF₃; —O—C(O)—R; —OSO₃⁻; —OPO₃²⁻; —OSO₃H; —OPO₃H₂; —OPO₃R₂; wherein each occurrence of R independently represents a hydrogen atom, or an optionally substituted linear, branched or cyclic C₁₋₆alkyl radical;
R⁴ may represent a linear, branched or cyclic C₁₋₆ alkyl group. Advantageously, R⁴ may represent methyl or ethyl; and
R⁵ may represent a hydrogen atom; a linear or branched C₁₋₆ alkyl group; —OH; F; Cl; Br; I; —CF₃; —NO₂; —OR′ wherein R′ represents a hydrogen atom or an optionally substituted linear, branched or cyclic C₁₋₆ alkyl or C₁₋₆ haloalkyl group; or —NR^{c}R^{d} wherein R^{c} et R^{d} independently represent H, a linear, branched or cyclic C₁₋₆ alkyl group, R^{a} et R^{b} and where R^{c} and R^{d} can form, together with the nitrogen atom to which they are attached, a heterocycle with 5 or 6 ring members. Advantageously, R⁵ may represent a hydrogen atom; —OH; F; Cl; Br; I; —CF₃; —NH₂; —OR′ wherein R′ represents methyl or ethyl. Advantageously, R⁵ may represent a hydrogen atom; Cl; Br; or —NH₂.

Advantageously, the compound according to the invention may have one of the following structures:

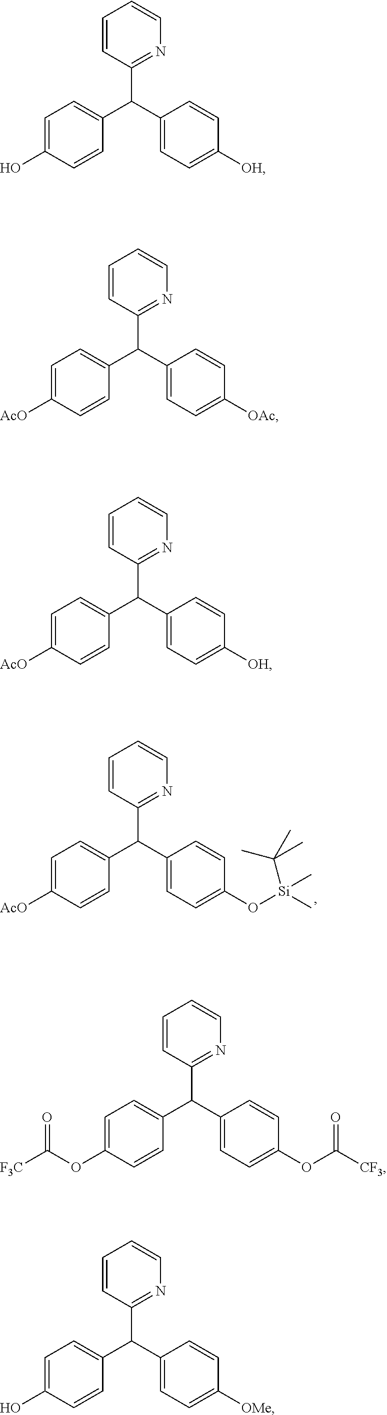 [SMILES: Oc1ccc(C(c2ccc(O)cc2)c2ccccn2)cc1 ; CC(=O)Oc1ccc(C(c2ccc(OC(C)=O)cc2)c2ccccn2)cc1 ; CC(=O)Oc1ccc(C(c2ccc(O)cc2)c2ccccn2)cc1 ; CC(=O)Oc1ccc(C(c2ccc(O[Si](C)(C)C(C)(C)C)cc2)c2ccccn2)cc1 ; O=C(Oc1ccc(C(c2ccc(OC(=O)C(F)(F)F)cc2)c2ccccn2)cc1)C(F)(F)F ; COc1ccc(C(c2ccc(O)cc2)c2ccccn2)cc1]

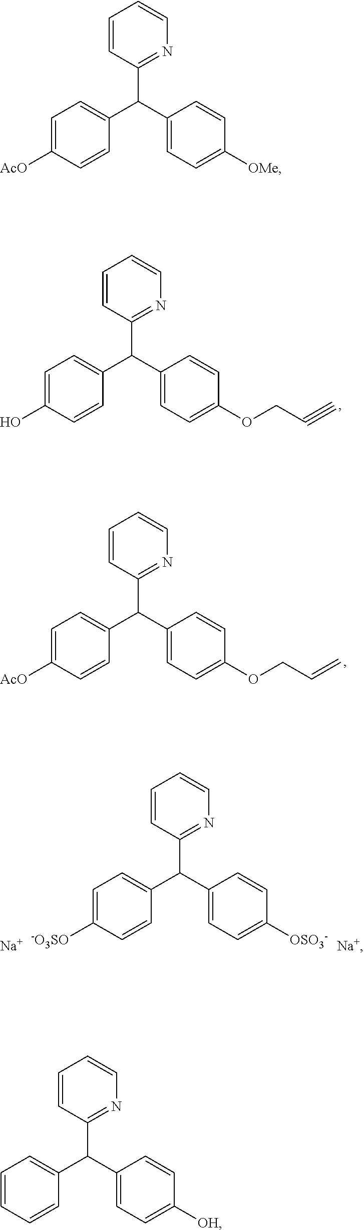

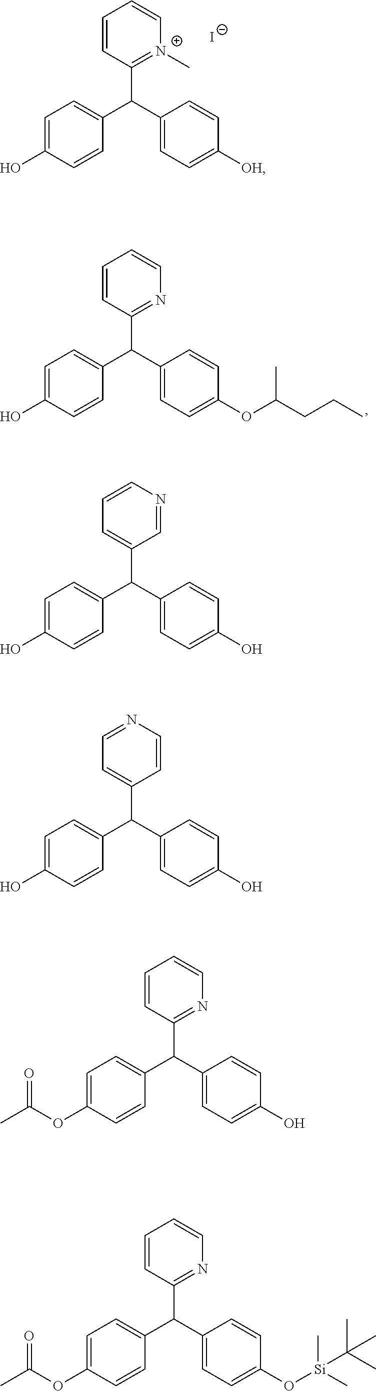

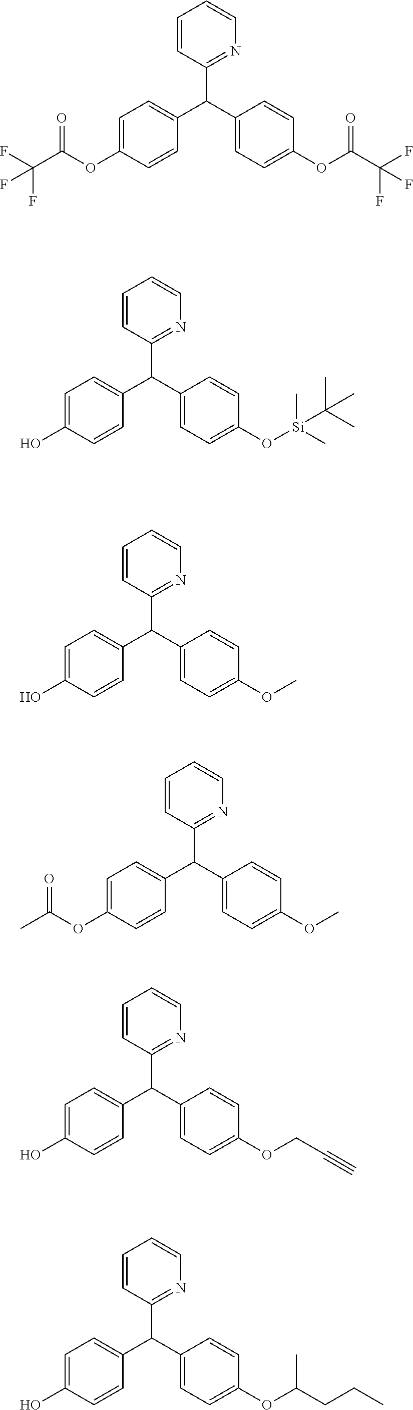

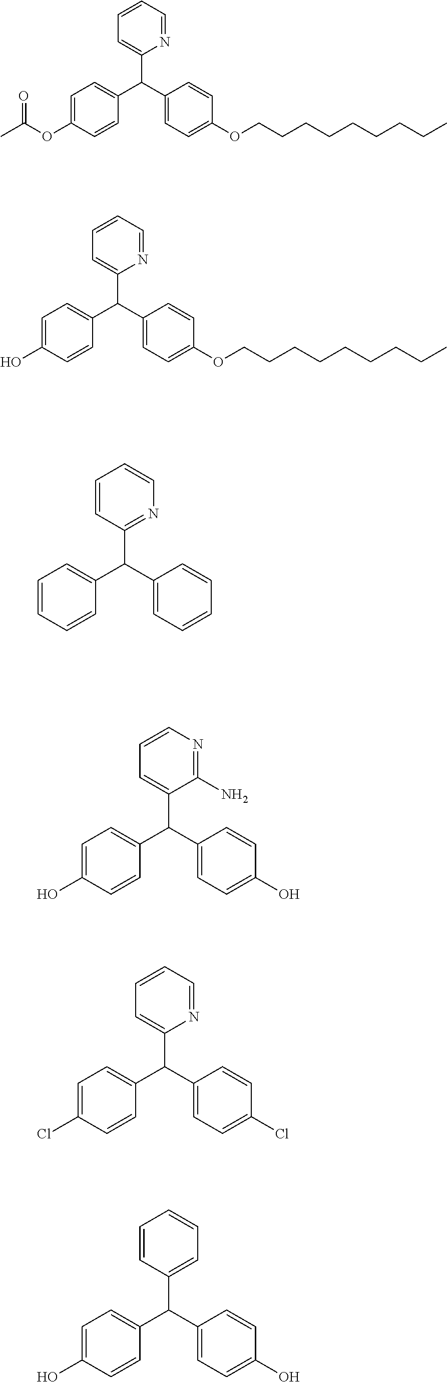 [SMILES: CCCCCCCCCOc1ccc(C(c2ccc(OC(C)=O)cc2)c2ccccn2)cc1 ; CCCCCCCCCOc1ccc(C(c2ccc(O)cc2)c2ccccn2)cc1 ; c1ccc(C(c2ccccc2)c2ccccn2)cc1 ; Nc1ncccc1C(c1ccc(O)cc1)c1ccc(O)cc1 ; Clc1ccc(C(c2ccc(Cl)cc2)c2ccccn2)cc1 ; Oc1ccc(C(c2ccccc2)c2ccc(O)cc2)cc1]

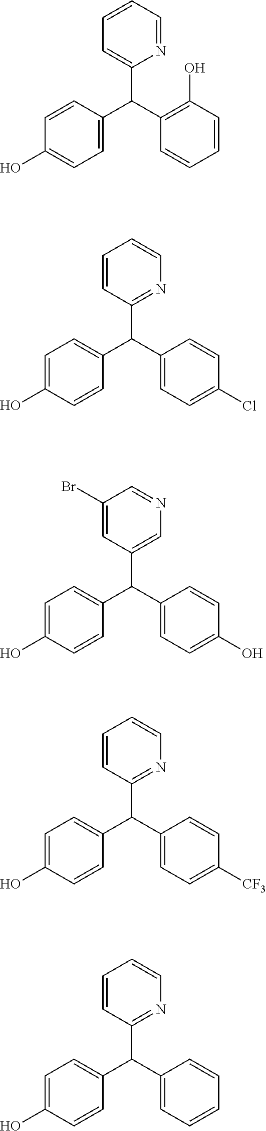 [SMILES: Oc1ccc(C(c2ccccn2)c2ccccc2O)cc1 ; Oc1ccc(C(c2ccc(Cl)cc2)c2ccccn2)cc1 ; Oc1ccc(C(c2ccc(O)cc2)c2cncc(Br)c2)cc1 ; Oc1ccc(C(c2ccc(C(F)(F)F)cc2)c2ccccn2)cc1 ; Oc1ccc(C(c2ccccc2)c2ccccn2)cc1]

or a pharmaceutically acceptable salt thereof.

Advantageously, the compound may be bisacodyl or its metabolite 4,4′-(dihydroxy-diphenyl)-(2-pyridyl)methane (DDPM).

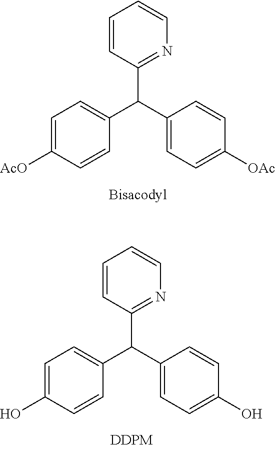 [SMILES: CC(=O)Oc1ccc(C(c2ccc(OC(C)=O)cc2)c2ccccn2)cc1 ; Oc1ccc(C(c2ccc(O)cc2)c2ccccn2)cc1]

Advantageously, compounds of the invention may be active in the acidic conditions found within tumors. Advantageously, compounds of the invention may exhibit differential activity at acidic pH versus basic pH. Advantageously, compounds of the invention may exhibit higher activity in acidic conditions, and may therefore be selective for action and/or exhibit a greater efficacy in cells characterized by an acidic microenvironment, such as cancer stem cells. In general, it is known that the intratumor microenvironment is on average more acidic than normal cells' microenvironment (Song et al., Cancer Drug Discovery and Development: Cancer Drug resistance”, Chapter 2, pp. 21-42 (2006) (72)). A pH exists within the tumor, with pH values that can be as low as 5.8-6.3.

The term “intratumor microenvironment” as used herein refers to a complex system of many cells, which all participate in tumor progression, including mesenchymal cells, endothelial cells and their precursors, pericytes, smooth-muscle cells, fibroblasts of various phenotypes, myofibroblasts, neutrophils and other granulocytes (eosinophils and basophils), mast cells, T, B and natural killer lymphocytes, and antigen-presenting cells such as macrophages and dendritic cells. The components of the intratumor microenvironment can be grouped into four categories: Cancer cells, Non-cancer cells, Secreted soluble factors, and Non-cellular solid material, including the extra-cellular matrix.

Advantageously, compounds of the invention may be active specifically in acidic conditions, that is at pH<7. The pH-dependent differential activity is an important feature as it allows to specifically target certain types of cells characterized by an acidic microenvironment. That is the case for cancer cells in general (intratumor microenvironment), and cancer stem cells more specifically.

The ability to selectively act on cancer stem cells is important because these cells are more resistant to conventional treatments than cancer cells that are more differentiated. This resistance can be increased when cancer stem cells are in quiescent state. A cancerous mass is considered as a kind of organoid that presents a cellular heterogeneity and plasticity. Specifically, all cancer cells within a tumor are not in the same state, and all are not in proliferation. Tumors contain cancer stem cells, which, depending on their microenvironment and diverse stimulations, can oscillate between a quiescent state and a proliferative state. One of the reasons that cancer stem cells are resistant to conventional cancer treatments is specifically because they can exist in a quiescent state, which allows them to be immune to drugs acting on cells in proliferation. Thus, these cancer stem cells in quiescent state, which remain intact and unaffected after conventional cancer treatments, are one underlying source/reason behind cancer recurrence (the tumor disappears, then reappears).

Another important aspect associated with the pH-dependent differential activity of compounds of the invention is the fact that the intratumor microenvironment is on average naturally acidic. This natural acidity creates an intratumor microenvironment that can protect cancer cells from certain drugs, notably those drugs that unstable and/or lose activity in acidic medium.

Advantageously, compounds of the invention may exhibit cytotoxic activity at the natural pH gradient existing in the intratumor microenvironment, for example at pH 5.0-6.9, for example 5.5-6.9, for example 5.8-6.9, for example 5.8-6.8, for example 6.0-6.8, for example about 6.6, or for example 5.8-6.3.

Advantageously, compounds of the invention remain cytotoxic to quiescent cancer stem cells.

In another aspect, there is provided a pharmaceutical composition for treating cancer, comprising a therapeutically effective amount of any one or more of the compounds described herein, or pharmaceutically acceptable derivative thereof, and a pharmaceutically acceptable carrier, adjuvant, or vehicle.

Advantageously, the compound may be in an amount to detectably exhibit cytotoxic activity towards proliferating and/or quiescent cancer stem cells.

Advantageously, the pharmaceutical composition may possess cytotoxicity to quiescent cancer stem cells.

Advantageously, these compositions optionally further comprise one or more additional therapeutic agents.

Advantageously, the pharmaceutical composition may additionally comprise a therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating destructive bone disorders, an agent for treating liver disease, an anti-viral agent, an agent for treating blood disorders, an agent for treating diabetes, or an agent for treating immunodeficiency disorders.

Advantageously, the additional therapeutic agent may be an anti-proliferative agent.

### DEFINITIONS

It is understood that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic, carbon and heteroatom substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment and prevention, for example of disorders, as described generally above. Examples of substituents include, but are not limited to alkyl; alkene, alkyne, cycloalkyl, cycloalkene, cycloalkyne, heteroalkyl; haloalkyl; aryl; heteroaryl; heterocycle; alkaryl; alkylheteroaryl; alkoxy; aryloxy; heteroalkoxy; heteroaryloxy; heteroalkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; — or a function -GR^{G1} in which G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, NR^{G2}C(═S)—, —SC(═S)NR^{G2}—, —NR^{G2}C(═S)S—, —NR^{G2}C(═S)NR^{G2}—, —SC(═NR^{G2})—, —C(═S)NR^{G2}—, —OC(═S)NR^{G2}—, —NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O— or —SO₂NR^{G2}—, where each occurrence of R^{G1}, R^{G2} and R^{G3} is, independently of the other occurrences of R^{G1}, a hydrogen atom; a halogen atom; or an optionally substituted linear, branched or cyclic alkyl, heteroalkyl, alkene or alkyne function; or an aryl, heteroaryl, heterocycle, alkaryl or alkylheteroaryl group in which the aryl, heteroaryl or heterocyclic radical is optionally substituted; or else, when G represents —NR^{G2}—, R^{G1} and R^{G2}, together with the nitrogen atom to which they are attached, form a heterocycle or a heteroaryl, optionally substituted.

Additional examples of generally applicable substituents are illustrated by the specific embodiments shown in the Examples that are described herein.

The term “stable”, as used herein, preferably refers to compounds which possess stability sufficient to allow manufacture and which maintain the integrity of the compound for a sufficient period of time to be characterized and detected, and preferably (but not necessarily) for a sufficient period of time to be useful for the medical purposes detailed herein. For purposes of the present description, “stable” compounds encompass pharmaceutically acceptable derivatives as defined below, such as pro-drugs, which exhibit sufficient stability to allow manufacture, and preferably storage and formulation, but are transformed (e.g., hydrolyzed) into a compound as otherwise described herein, or a metabolite or residue thereof, for example when administered to a subject, or manipulated/tested in in vitro assays, such as cell-based assays.

“Halo” or “halogen” as used herein denotes an atom selected from fluorine, chlorine, bromine and iodine.

The alkyl radicals can comprise from 1 to 18 carbon atoms, notably from 1 to 12 carbon atoms, and in particular from 1 to 6 carbon atoms.

The alkenyl radicals can comprise from 2 to 18 carbon atoms, notably from 2 to 12 carbon atoms, and in particular from 2 to 6 carbon atoms. They can moreover comprise one or more double bond(s).

The alkynyl radicals can comprise from 2 to 18 carbon atoms, notably from 2 to 12 carbon atoms, and in particular from 2 to 6 carbon atoms. They can moreover comprise one or more triple bond(s).

Unless stated otherwise, the alkyl, alkenyl and alkynyl radicals can be linear, branched or cyclic.

The term “heteroalkyl” denotes an alkyl radical in which at least one carbon atom in the main chain has been replaced with a heteroatom. Thus, a heteroalkyl denotes an alkyl radical comprising, in its main chain, at least one heteroatom selected from nitrogen, sulphur, phosphorus, silicon, oxygen or selenium atoms in place of a carbon atom. Thus, a C₁₋₆ heteroalkyl radical denotes a radical comprising 1 to 6 carbon atoms and at least one heteroatom selected from the nitrogen, sulphur, phosphorus, silicon, oxygen or selenium atoms.

The term “aryl” denotes a mono-, bi- or tricyclic hydrocarbon system comprising one, two or three rings satisfying Hückel's aromaticity rule. For example, an aryl radical can be a phenyl, naphthyl, tetrahydronaphthyl, indanyl, indenyl group and similar radicals. The aryl radicals can comprise from 6 to 14 carbon atoms and notably from 6 to 10 carbon atoms.

The term “heteroaryl” denotes an unsaturated heterocyclic system comprising at least one aromatic ring, and from 5 to 14 ring members, among which at least one group of the cyclic system is selected from S, O and N; zero, one or two ring members of the cyclic system are additional heteroatoms selected independently of one another from S, O and N; the remaining ring members of the cyclic system being carbon atoms; the heteroaryl radical being bound to the rest of the molecule via any one of the ring members of the cyclic system (whether it is a carbon atom or a heteroatom). For example, a heteroaryl radical can be a pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl radical, and similar radicals.

The aralkyl and alkaryl radicals can comprise from 7 to 25 carbon atoms, notably from 7 to 20 carbon atoms and in particular from 7 to 15 carbon atoms. Quite particularly the alkaryl radical can represent a benzyl.

The heteroaralkyl and alkylheteroaryl radicals can comprise from 7 to 25 carbon atoms, notably from 7 to 20 carbon atoms and in particular from 7 to 15 carbon atoms.

The term “heterocycle” denotes a mono- or polycyclic, saturated or unsaturated, non-aromatic cyclic system comprising 5 to 20 ring members, and optionally comprising one or more rings with 5 or 6 ring members having between 1 and 3 heteroatoms selected independently of one another from S, O, N, P, Se and Si in which (i) each ring with 5 ring members has from 0 to 2 double bonds, and each ring with 6 ring members has from 0 to 2 double bonds, (ii) the sulphur and/or nitrogen atoms are optionally oxidized, and (iii) the nitrogen atoms are optionally in the form of quaternary salt. For example, a heterocyclic radical can be a pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, or tetrahydrofuryl group.

A heterocycle comprises in its cyclic system, besides carbon atoms, at least one heteroatom, notably selected from oxygen, nitrogen, sulphur, phosphorus, selenium and silicon.

The term “amine” or “amino” denotes a radical corresponding to the formula —N(R)₂ in which each occurrence of R is, independently of one another, a hydrogen atom; an alkyl, heteroalkyl, alkene, alkyne, aryl, heteroaryl, aralkyl, alkaryl, heteroaralkyl, alkylheteroaryl radical, optionally substituted; or in which the groups R form, with the nitrogen atom to which they are attached, a heterocycle or heteroaryl, optionally substituted. The amine function can optionally be in the form of a quaternary amine salt.

As used herein, the term “isolated”, when applied to the compounds of the present invention, refers to such compounds that are (i) separated from at least some components with which they are associated in nature or when they are made and/or (ii) produced, prepared or manufactured by the hand of man.

As used herein, the term “treat” or “treatment” generally means that the compounds or compositions of the invention can be used in humans or animals in a therapeutic or prophylactic application with at least one attempt to diagnose the disease. For example, the compounds or compositions of the invention can delay, slow, inhibit, promote or induce one or more target biological processes implicated or associated with the disease to be treated. For example, the compounds or compositions of the invention can delay or slow the progression of the disease, or prevent it.

As used herein, the term “prevention” or “prevent” means that the compounds or compositions of the present invention are useful when they are administered to a patient who has not been diagnosed as possibly having the disease at the time of administration, but who is likely to develop the disease or has an increased risk of developing the disease. For example, the compounds or compositions of the invention can slow the development of symptoms of the disease, delay the appearance of the disease, or prevent the individual developing the disease. The term “prevention” or “prevent” also comprises the administration of the compounds or compositions of the invention to subjects who may be predisposed to the disease, based on family history, genetic or chromosomal abnormalities, and/or owing to the presence of one or more biological markers of the disease.

As used herein, “cancer stem cells” means cancer cells displaying certain properties of stem cells of the original tissue, but also a mesenchymal molecular profile. Typically, the cancer stem cells are capable of forming a tumour after grafting in the corresponding organ or ectotopically (a few cells is sufficient (just one ideally, less than 100 in practice)). For example, a graft of cancer stem cells of glioblastoma in the brain of immunodeficient mice led to a model where a minority population, with properties that are stable and different from the other tumour cells (designated in the literature “tumour initiating cells” or TIC), is at the origin of the tumour and of its resistance to treatments. The concept of TICs present in a small amount and situated at the peak of the hierarchy of the cells making up the tumour was proposed to be at the origin of leukaemias. TICs have now been isolated from several types of solid tumours including gliomas, which constitute the majority of primitive tumours of the central nervous system. Stricto sensu, the term “cancer stem cells” would refer to stem cells that become cancerous. However, it is conceivable that cancerous stem cells found in tumors may originate from another cell type which has become cancerous and has acquired stem cell properties. It is known that the intratumor microenvironment, and in particular hypoxia and the acidic environment of tumoral cells, favors the formation of stem cells. Because the cell type from which “cancer stem cells” originate is not known, scientists often refer to them as “cancer stem-like cells”. The terms “cancer stem cells”, “cancer initiating cells”, “cancer propagating cells”, and “cancer stem-like cells” are used interchangeably to mean the same thing. For purposes of the present description, the term “cancer stem cells” is meant to cover all types of cancer stem cells referred to above, independently of their origin, which all share a common functional definition: cancer cells that possess characteristics associated with normal stem cells, specifically the ability of self-renewal, differentiation into multiple cell types, and to induce tumors in xenografts (or xenotransplants).

For example, the reader can refer to publications [12] through [36] describing the isolation/identification of cancer stem cells from various tumours.

In the present text, the term “quiescent cells” refers to cells in the G0/G1 phase of the cell cycle for which the biological process of cell division has stopped for the time being. Quiescent cells are by definition arrested in the cell cycle and their metabolic needs are decreased. For example, they may be quiescent cancer cells.

The term “cancer associated with cancer stem cells in quiescent state”, as used herein, means any cancer in which cancer stem cells in quiescent state are present. For example, these include cancers such as brain, ovarian, liver, breast cancer and melanoma.

The term “treating”, as used herein generally means that the compounds of the invention can be used in humans or animals with at least a tentative diagnosis of disease. Advantageously, compounds of the invention will delay or slow the progression of the disease thereby giving the individual a longer life span.

The term “preventing” as used herein means that the compounds of the present invention are useful when administered to a patient who has not been diagnosed as possibly having the disease at the time of administration, but who would normally be expected to develop the disease or be at increased risk for the disease. The compounds of the invention will slow the development of disease symptoms, delay the onset of disease, or prevent the individual from developing the disease at all. Preventing also includes administration of the compounds of the invention to those individuals thought to be predisposed to the disease due to familial history, genetic or chromosomal abnormalities, and/or due to the presence of one or more biological markers for the disease.

As used herein the term “biological sample” includes, without limitation, cell cultures or extracts thereof; biopsied material obtained from an animal (e.g., mammal) or extracts thereof; and blood, saliva, urine, feces, semen, tears, or other body fluids or extracts thereof. For example, the term “biological sample” refers to any solid or fluid sample obtained from, excreted by or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). The biological sample can be in any form, including a solid material such as a tissue, cells, a cell pellet, a cell extract, cell homogenates, or cell fractions; or a biopsy, or a biological fluid. The biological fluid may be obtained from any site (e.g. blood, saliva (or a mouth wash containing buccal cells), tears, plasma, serum, urine, bile, cerebrospinal fluid, amniotic fluid, peritoneal fluid, and pleural fluid, or cells therefrom, aqueous or vitreous humor, or any bodily secretion), a transudate, an exudate (e.g. fluid obtained from an abscess or any other site of infection or inflammation), or fluid obtained from a joint (e.g. a normal joint or a joint affected by disease such as rheumatoid arthritis, osteoarthritis, gout or septic arthritis). The biological sample can be obtained from any organ or tissue (including a biopsy or autopsy specimen) or may comprise cells (whether primary cells or cultured cells) or medium conditioned by any cell, tissue or organ. Biological samples may also include sections of tissues such as frozen sections taken for histological purposes. Biological samples also include mixtures of biological molecules including proteins, lipids, carbohydrates and nucleic acids generated by partial or complete fractionation of cell or tissue homogenates. Although the sample is preferably taken from a human subject, biological samples may be from any animal, plant, bacteria, virus, yeast, etc. The term animal, as used herein, refers to humans as well as non-human animals, at any stage of development, including, for example, mammals, birds, reptiles, amphibians, fish, worms and single cells. Cell cultures and live tissue samples are considered to be pluralities of animals. Advantageously, the non-human animal may be a mammal (e.g., a rodent, a mouse, a rat, a rabbit, a monkey, a dog, a cat, a sheep, cattle, a primate, or a pig). An animal may be a transgenic animal or a human clone. If desired, the biological sample may be subjected to preliminary processing, including preliminary separation techniques.

The compounds described in the present text can have one or more asymmetric centres, and can therefore exist in various isomeric forms, for example as stereoisomers and/or diastereoisomers. For example, when R¹ and R² are different, the compounds of formula I can exist in the form:

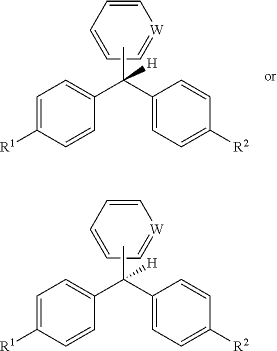

in which W, R¹ and R² are as defined above.

Thus, the compounds of the invention can be in the form of an enantiomer, diastereoisomer or geometric isomer, or can be in the form of a mixture of stereoisomers, for example a racemic mixture. The compounds described in the present text can be enantiopure compounds. The compounds described in the present text can be in the form of mixtures of stereoisomers or diastereoisomers.

Synthetic Overview

A person skilled in the art has at his disposal a well-established literature on the chemistry of bisacodyl that can be utilized, in combination with the information contained in the present text, to obtain instruction on the synthesis strategies, notably the protecting groups, and other materials and methods useful for synthesis of the compounds described in the present text.

The various references cited in the present text supply general information useful for preparation of the compounds according to the invention or of relevant intermediates.

For example, information can be found in Pala et al., (1968) Tetrahedron, 24(2), pp. 619-624 [11].

A synthesis strategy applied for preparing the condensation compounds according to the invention is illustrated by methods A, B, C and D described below.

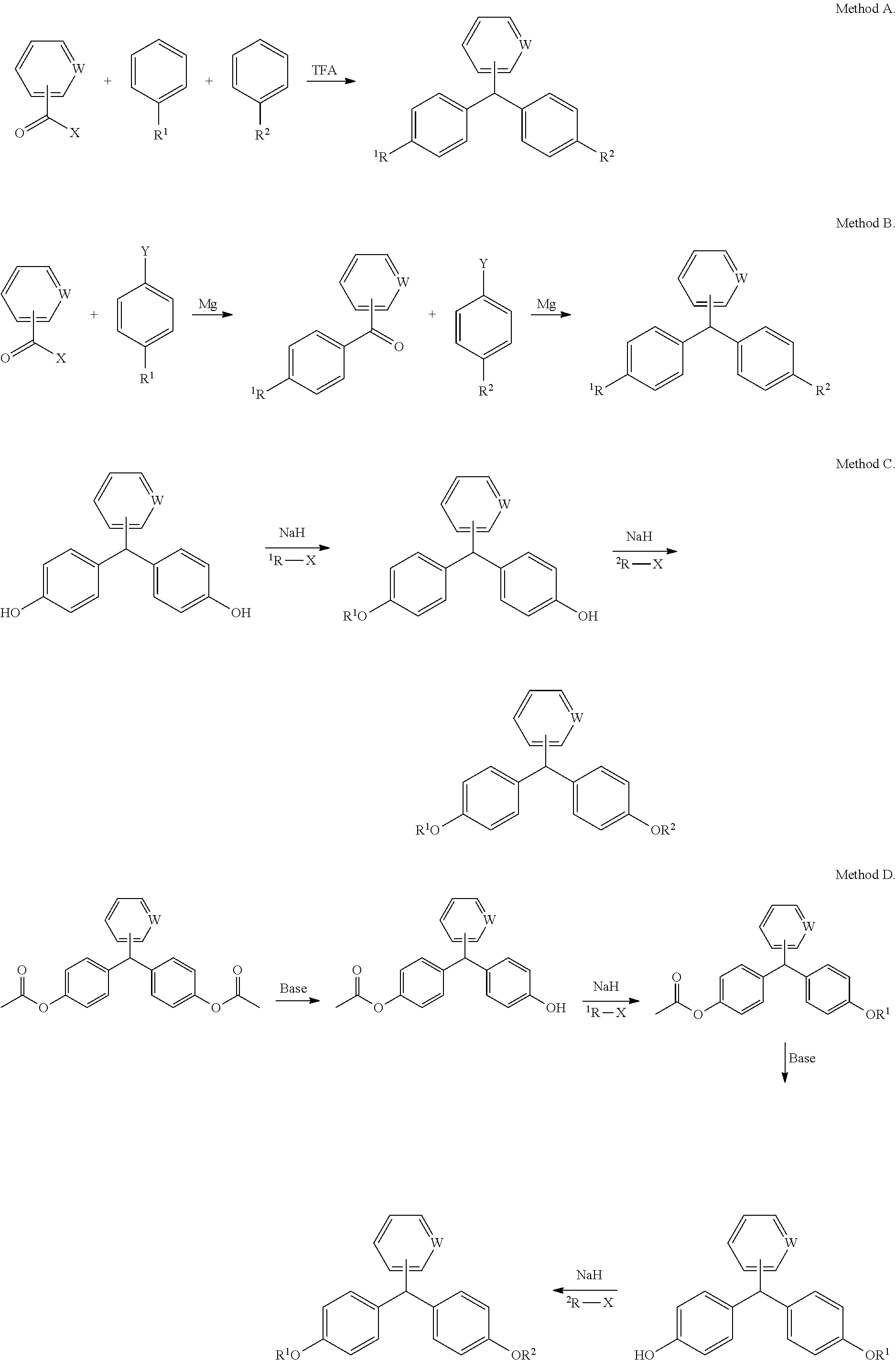

The above methods are illustrated in the Examples, notably in the section “Synthesis of the compounds”, under the heading “A. General methods of synthesis of the compounds according to the invention”.

Numerous suitable prodrug radicals, and information concerning the selection, synthesis and use thereof, are well known in the prior art. Examples of prodrug radicals of interest comprise, among others, the prodrug radicals that can be attached to groups containing a primary or secondary amine. For example, they may be prodrug radicals that can be attached to an —NH₂ group. The following examples of these prodrug radicals may be mentioned:

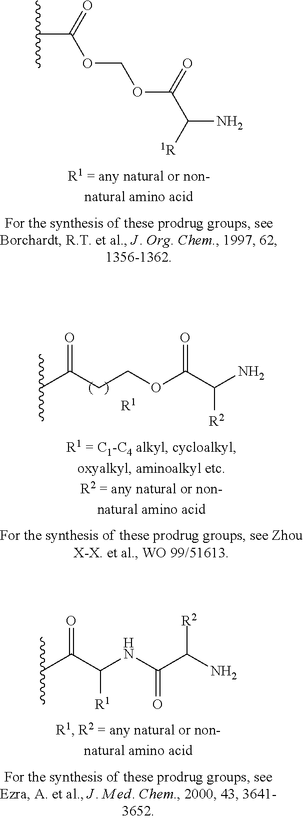

The ester, phosphate, and sulphate groups are also prodrug radicals. Thus, the compounds of formula I in which R¹ or R² represents —O—C(O)—R, —OSO₃⁻; —OPO₃²⁻; —OSO₃H; —OPO₃H₂, where R can represent a C₁₋₆ alkyl group, can serve as the basis for a compound according to the invention in the form of a prodrug.

The present invention includes any form of prodrugs of the compounds described in the present text. The examples of prodrug radicals described above are given for purposes of illustration and are non-limiting.

2/Pharmaceutical Compositions

According to another of its aspects, the invention also relates to a pharmaceutical composition comprising at least one compound or at least one pharmaceutically acceptable salt thereof as defined previously.

According to another aspect, the invention also relates to a compound according to the invention or a pharmaceutically acceptable salt thereof, as a pharmaceutical composition intended for treating cancer, regardless of its nature and its degree of anaplasia.

In another aspect, there is provided a pharmaceutical composition for treating cancer, comprising a therapeutically effective amount of any one or more of the compounds described herein, or pharmaceutically acceptable derivative thereof, and a pharmaceutically acceptable carrier, adjuvant, or vehicle.

Advantageously, the compound may be in an amount to detectably exhibit cytotoxic activity towards proliferating and/or quiescent cancer stem cells.

Advantageously, the pharmaceutical composition may possess cytotoxicity towards quiescent cancer stem cells.

Advantageously, these compositions optionally further comprise one or more additional therapeutic agents.

Advantageously, the pharmaceutical composition may additionally comprise a therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating destructive bone disorders, an agent for treating liver disease, an anti-viral agent, an agent for treating blood disorders, an agent for treating diabetes, or an agent for treating immunodeficiency disorders.

Advantageously, the additional therapeutic agent may be an anti-proliferative agent.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof.

“Pharmaceutically acceptable salts” means, in the sense of the present invention, salts suitable for pharmaceutical use. They may be salts which are, in a medical context, suitable for a use involving contact with tissues (human or animal) without causing notable toxicity, irritation or allergic response, and have a reasonable benefit/risk ratio. For example, “pharmaceutically acceptable salt” can be any non-toxic salt or a salt of an ester of a compound of the present invention which, when administered to a subject, is capable of supplying, directly or indirectly, a compound according to the present invention or an active metabolite or a residue of the latter. In the present text, “active metabolite or a residue of the latter” means a metabolite or a residue of the latter that also displays antitumour activity.

The pharmaceutically acceptable salts are well known, and can be obtained by techniques that are well known by a person skilled in the art. As an example, we may mention S. M. Berge et al., J. Pharmaceutical Sciences, 1977, 66, 1-19, which is incorporated herein by reference, and which describes pharmaceutically acceptable salts in detail.

The pharmaceutically acceptable salts of the compounds described in the present text comprise those derived from suitable organic acids and inorganic bases. Examples of pharmaceutically acceptable and non-toxic salts of acid addition include of salts of an amino group formed with inorganic acids such as hydrochloric acid, hydrobromic acid, phosphoric acid, sulphuric acid and perchloric acid or with organic acids such as acetic acid, oxalic acid, maleic acid, tartaric acid, citric acid, succinic acid or malonic acid or using other methods used in the art, such as ion exchange. Other pharmaceutically acceptable salts comprise adipate, alginate, ascorbate, aspartate, benzenesulphonate, benzoate, bisulphate, borate, butyrate, camphorate, camphorsulphonate, citrate, cyclopentanepropionate, digluconate, dodecylsulphate, ethanesulphonate, formate, fumarate, glucoheptonate, glycerophosphate, gluconate, hemisulphate, heptanoate, hexanoate, hydriodide, 2-hydroxy-ethanesulphonate, lactobionate, lactate, laurate, lauryl sulphate, malate, maleate, malonate, methanesulphonate, 2-naphthalene, nicotinate, nitrate, oleate, oxalate, palmitate, palmoate, pectinate, persulphate, 3-phenylpropionate, phosphate, picrate, pivalate, propionate, stearate, succinate, sulphate, tartrate, thiocyanate, p-toluenesulphonate, undecanoate, valerate salts, etc.

The pharmaceutically acceptable salts derived from suitable bases comprise alkali metal, alkaline-earth, and ammonium salts. Quaternary salts of any basic group containing a nitrogen atom present in the compounds described in the present text are also included. Products that are soluble or dispersible in water or oil can be obtained by said quaternization of basic groups containing a nitrogen atom. As examples of alkali metal or alkaline-earth salts we may mention sodium, lithium, potassium, calcium, magnesium, etc. Moreover, the pharmaceutically acceptable salts comprise, if applicable, the non-toxic cations of ammonium, of quaternary ammonium, and of amine formed with counter-ions such as a halide, a hydroxide, a carboxylate, a sulphate, a phosphate, a nitrate, an alkylsulphonate or arylsulphonate group.

Advantageously, the compound according to the invention or at least one pharmaceutically acceptable salt thereof can be present in the pharmaceutical composition in an amount in the range from 1 to 400 mg per unit dose, and in particular from 10 to 40 mg.

Advantageously, the pharmaceutical composition can comprise an amount of at least one compound or of at least one pharmaceutically acceptable salt thereof in the range from 1 to 100 mg, in particular from 10 to 40 mg.

The pharmaceutical composition can further comprise a pharmaceutically acceptable carrier.

“Pharmaceutically acceptable carrier” means, in the sense of the present invention, a substance that is suitable for use in a pharmaceutical product.

Thus, the pharmaceutically acceptable compositions of the present invention can further comprise a pharmaceutically acceptable carrier, additive, or vehicle, which, as is to be understood in the present text, comprises any solvent, diluent, or other liquid vehicle, dispersing or suspending agent, surfactant, isotonic agent, thickener or emulsifier, preservative, solid binder, lubricant and others, that is suitable for the particular dosage form required. Remington's Pharmaceutical Sciences, twentieth edition, E W Martin (Mack Publishing Co., Easton, Pa., 2000) describes various carriers used in the formulation of pharmaceutically acceptable compositions and known techniques for preparing them. Any conventional pharmaceutical vehicle can be used in the context of the present invention, unless it is incompatible with the compounds of the invention, for example if it produces an undesirable biological effect or else if it interacts adversely with another component of the pharmaceutical composition. Some examples of materials that can serve as pharmaceutically acceptable vehicles comprise, but are not limited to, ion exchangers, alumina, aluminium stearate, lecithin, serum proteins such as human serum albumin, buffer substances such as phosphates, glycine, sorbic acid or potassium sorbate, mixtures of partial glycerides of saturated vegetable fatty acids, water, salts or electrolytes, such as protamine sulphate, disodium phosphate, potassium hydrogen phosphate, sodium chloride, zinc salts, colloidal silica, magnesium trisilicate, polyvinylpyrrolidone, polyacrylates, waxes, ethylene-polyoxypropylene polymers, wool fat, sugars such as lactose, glucose and sucrose; starches such as maize starch and potato starch, cellulose and its derivatives such as sodium carboxymethylcellulose, ethylcellulose and cellulose acetate; tragacanth in powder form, malt; gelatin; talc; excipients such as cocoa butter and waxes for suppositories; oils such as peanut oil, cottonseed oil, safflower oil, sesame oil, olive oil, maize oil and soya oil; glycols such as propylene glycol or polyethylene glycol, esters such as ethyl oleate and ethyl laurate; agar; buffers such as magnesium hydroxide and aluminium hydroxide, alginic acid, apyrogenic water; isotonic saline, Ringer solution, ethyl alcohol, and phosphate buffer solutions, as well as other compatible, non-toxic lubricants, such as sodium lauryl sulphate and magnesium stearate, as well as colorants, stripping agents, coating agents, sweeteners, flavourings and perfumes, preservatives and antioxidants.

The pharmaceutical composition can comprise a content of pharmaceutically acceptable carrier in the range from 5 to 99 wt. %, notably from 10 to 90 wt. %, and in particular from 20 to 75 wt. % relative to the total weight of the composition.

The pharmaceutical compositions according to the invention can be in various forms, notably in a form selected from the group comprising tablets, capsules, coated tablets, syrups, suspensions, solutions, powders, granules, emulsions, microspheres and injectable solutions and solid lipid nanoparticles.

These various forms can be obtained by techniques that are well known by a person skilled in the art.

Quite particularly the formulations suitable for administration by the parenteral route, the pharmaceutically acceptable vehicles suitable for this route of administration and the corresponding techniques for formulation and administration can be carried out according to methods that are well known by a person skilled in the art, in particular those described in the manual Remington's Pharmaceutical Sciences (Mack Publishing Co., Easton, Pa., 20th edition, 2000).

The liquid dosage forms for oral administration comprise, but are not limited to, emulsions, microemulsions, solutions, suspensions, syrups and elixirs. In addition to the active compounds, the liquid pharmaceutical forms can contain inert diluents commonly used in the art such as, for example, water or other solvents, solubilizers and emulsifiers such as ethyl alcohol, isopropyl alcohol, ethyl carbonate, ethyl acetate, benzyl alcohol, benzyl benzoate, propylene glycol, 1,3-butylene, dimethylformamide, oils (in particular cottonseed oil, peanut oil, maize oil, wheat germ oil, olive oil, castor oil, and sesame oil), glycerol, tetrahydrofurfuryl alcohol, polyethylene glycols and sorbitan fatty acids, and mixtures thereof. Besides the inert diluents, the oral compositions can also comprise additives such as wetting, emulsifying and suspending agents, sweeteners, flavourings and perfumes.

The injectable preparations, for example aqueous or oily injectable sterile suspensions, can be formulated according to methods known in this field, using dispersants or wetting agents and suspending agents. The injectable sterile preparation can also be an injectable sterile solution, suspension or emulsion, in a non-toxic diluent or solvent that is acceptable for administration by the parenteral route, such as a solution in 1,3-butanediol for example. Among the acceptable vehicles and solvents that can be used we may mention water, Ringer solution, and an isotonic solution of sodium chloride. Moreover, the sterile fixed oils are used conventionally as solvent or suspending medium. For this purpose, any mild fixed oil can be used including the synthetic mono- and diglycerides. Moreover, fatty acids such as oleic acid can be used in the preparation of injectable products.

The injectable formulations can be sterilized, for example by filtration through a bacteria-retaining filter, or by incorporation of sterilizing agents in the form of solid sterile compositions that can be dissolved or dispersed in sterile water or any other injectable sterile medium before use.

In order to prolong the effect of a compound according to the present invention, it may be desirable to slow the absorption of the compound counting from subcutaneous or intramuscular injection. This can be achieved by using a liquid suspension of crystalline or amorphous material with low solubility in water. The degree of absorption of the compound then depends on its dissolution rate, which in its turn may depend on the size of the crystals and on the crystalline form. Moreover, prolonged absorption of a compound administered parenterally can be achieved by dissolving or suspending the compound in an oily vehicle. Injectable forms can be produced by forming microencapsulated matrices of the compound in biodegradable polymers, such as polylactide-polyglycolide. The rate of release of the compound can be controlled depending on the ratio of the compound to the polymer, and the nature of the particular polymer used. Examples of other biodegradable polymers comprise poly(ortho-esters) and poly(anhydrides). Injectable forms can also be made by trapping the compound in liposomes or microemulsions that are compatible with living tissues.

The solid dosage forms for oral administration comprise capsules, tablets, pills, powders and granules. In these solid dosage forms, the active compound can be mixed with at least one inert, pharmaceutically acceptable excipient or carrier such as sodium citrate or dicalcium phosphate and/or a) fillers or extenders such as starches, lactose, sucrose, glucose, mannitol and silicic acid, b) binders such as, for example, carboxymethylcellulose, alginates, gelatin, polyvinylpyrrolidone, sucrose, and gum arabic, c) humectants such as glycerin, d) disintegrants such as agar-agar, calcium carbonate, potato starch or tapioca starch, alginic acid, certain silicates, and sodium carbonate, e) agents for delaying dissolution, such as paraffin, f) accelerators of absorption such as quaternary ammonium compounds, g) wetting agents, such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulphate, and mixtures thereof. In the case of capsules, tablets, or pills, the dosage form can also comprise buffers.

Similar solid compositions can also be used as fillers in soft or hard gelatin capsules using excipients such as lactose as well as high molecular weight polyethylene glycols, etc. The solid dosage forms tablets, coated tablets, capsules, pills, and granules can be prepared with coatings or shells such as enteric coatings and other coatings well known in the pharmaceutical field. The compositions can optionally contain opacifiers and can also be formulated so that they release the active principle(s) only, or preferably, in a certain section of the intestinal tract, optionally in a sustained manner. Examples of coating compositions that can be used comprise polymeric substances and waxes.

The dosage forms for topical or transdermal administration of a compound according to the present invention comprise ointments, pastes, creams, lotions, gels, powders, solutions, aerosols, products to be inhaled, patches or dissolvable microneedles. The active principle can be mixed in sterile conditions with a pharmaceutically acceptable vehicle and any preservative or buffer that may be necessary.

Moreover, the use of transdermal devices, which have the advantage of permitting controlled release of a compound to the organism, comes within the scope of the present invention. These pharmaceutical forms can be produced by dissolving or distributing the compound in a suitable medium. Agents that improve absorption can also be used for increasing the flow of the compound through the skin. The rate can be controlled either by using a membrane for controlling the rate of absorption or by dispersing the compound in a polymer matrix or a gel.

According to another aspect, the pharmaceutically acceptable compounds or compositions of the present invention can be used in combination therapy, i.e. the pharmaceutically acceptable compounds or compositions can be administered at the same time, before, or after, one or more other desired therapeutic agents or medical procedures. The particular combination of therapies (therapeutic agents or medical procedures) to be used in a combination therapy can take into account the compatibility of the therapy and/or of the desired medical procedure, and the therapeutic effect to be achieved. The combined therapies can aim at an effect for the same disease (for example, a compound according to the invention can be administered in combination with another agent used for treating cancer), or they can aim at different effects (for example, control of side-effects).

For example, other therapies, chemotherapeutic agents or antiproliferative agents can be combined or associated with the compounds of the present invention for treating proliferative diseases and cancer. Thus, the compositions according to the invention can further comprise an anticancer active principle different from the compound as defined in the present text.

Examples of treatments or anticancer agents that can be used in combination or in association with the compounds or compositions according to the present invention comprise surgery, radiotherapy (for example, gamma rays, neutron radiotherapy, electron-beam radiotherapy, proton beam therapy, curietherapy and systemic radioactive isotopes, to mention just a few), endocrinology, biological response modifiers (interferons, interleukins and tumour necrosis factor (TNF) to name just a few), hyperthermia and cryotherapy, agents that aim to attenuate side-effects (for example, corticoids, folic acid or derivatives, antiemetics) and other chemotherapeutic drugs, including, but not limited to, alkylating agents (mechlorethamine, chlorambucil, cyclophosphamide, melphalan, ifosfamide, dacarbazine, procarbazine, temozolomide (TMZ), busulfan), antimetabolites (methotrexate), antagonists of purines and pyrimidine antagonists (6-mercaptopurine, 5-fluorouracil, cytarabine, gemcitabine, tegafur, ftorafur, thioguanine, fludarabine), spindle poisons (vinblastine, vincristine, vinorelbine, paclitaxel, docetacel), podophyllotoxins (etoposide, irinotecan, topotecan), antibiotics (doxorubicin, bleomycin, mitomycin), nitrosoureas (carmustine, lomustine, fotemustine), inorganic ions (cisplatin, carboplatin, oxaliplatin), enzymes (asparaginase), proteasome inhibitor (bortézomib) and hormones (tamoxifen, leuprolide, flutamide and megestrol), Gleevec™, Adriamycin, dexamethasone and cyclophosphamide and monoclonal antibodies (bevacizumab, cetuximab, trastuzumab). For a more complete discussion of the latest cancer therapies, see The Merck Manual, seventeenth edition, 1999, the entire contents of which are incorporated by reference in the present text. See also the Internet site of the National Cancer Institute (NCI) (www.nci.nih.gov) and of the Food and Drug Administration (FDA) to obtain a list of medicinal products approved by the FDA in oncology (www.fda.gov/cder/cancer/druglistframe—See appendix).

Advantageously, compounds of the invention may be used in combination with the following drugs: temozolomide (TMZ), dacarbazine, fotemustine, docetaxel, oxaliplatin, cisplatin, Gemcitabine, 5-Fluorouracile, epirubicine, irinotecan.

The dose of each drug used in combination will be decided by the attending physician within the scope of sound medical judgment, and will advantageously be within conventional standards of care. The specific effective dose level for any particular subject will depend upon a variety of factors including the type of cancer being treated and its severity; the activity of the specific drug employed; the specific composition employed; the age, body weight, general health, sex and diet of the subject; the time of administration, route of administration, and rate of excretion of the specific compound employed; the duration of the treatment; drugs used in combination or coincidental with the specific compound employed, and like factors well known in the medical arts.

Advantageously, the pharmaceutical composition according to the invention can further comprise at least one anticancer agent selected from the group comprising:
Inhibitors of nucleic acid biosynthesis such as
   Antimetabolites: purine analogues (6-mercaptopurine, fludarabine, cladribine, pentostatin), pyrimidine analogues (cytarabine, 5-fluorouracil, gemcitabine), folic acid analogues (methotrexate, raltitrexed);
   Inhibitors of ribonucleotide reductase (hydroxycarbamide);
   Inhibitors of DNA topoisomerase I (irinotecan, topotecan);
   Inhibitors of DNA topoisomerase II (etoposide);
Substances reacting with DNA such as
   Intercalating substances such as anthracyclines (daunorubicin, doxorubicin, epirubicin, ibarubicin, pirarubicin), or anthracenediones (mitoxantrone);
   Electrophilic agents such as bifunctional alkylating agents (chlormethine, cyclophosphamide, ifosfamide, melphalan, chlorambucil, busulphan), nitro-ureas (carmustine, fotemustine, streptozocin), mitomycin C, platinum derivatives (carboplatin, cisplatin, oxaliplatin), related compounds (procarbazine, dacarbazine);
   Cleaving agents such as bleomycin;
substances interacting with proteins such as tubulin (alkaloid from periwinkle (vinblastine, vincristine, vindesine, vinorelbine), taxanes (paclitaxel, docetaxel) or asparagine (L asparagine);
inhibitors of angiogenesis such as angiostatin, endostatin, genistein, staurosporine, avastin and thalidomide;
antiproliferative agents such as N-acetyl-D-sphingosine, aloe-emodin, apigenin, berberine chloride, emodin, hydroxycholesterol and rapamycin;
agents inhibiting DNA synthesis such as amethopterin, cytosine β-D-arabinofuranoside, 5-fluoro-5-deoxyuridine, ganciclovir, hydroxyurea, mercaptopurine and thioguanine;
enzyme inhibitors such as DL-aminoglutethimide, apicidin, 2′,4′,3,4-tetrahydroxychalcone, camptothecin, deguelin, depudecin, doxycycline, etoposide, formestane, fostriecin, hispidin, indomethacin, mevinolin, oxamflatin, roscovitine, trichostatin and tyrphostin AG.

Advantageously, the pharmaceutical composition according to the invention can further comprise at least one anticancer agent selected from the group comprising: electrophilic agents (alkylating agents, nitroso-ureas, hydroxyurea, platinum derivatives), intercalating agents, cleaving agents, antimetabolites, enzyme inhibitors (inhibitors of topoisomerases, ribonucleotide reductases, tyrosine kinases, farnesyl transferases), integrin receptor inhibitors, monoclonal antibodies, agents acting on the mitotic spindle, inhibitors of histone deacetylases (HDACs), inhibitors of Akt signalling, Notch signalling, Sonic Hedgehog signalling. They can be for example temozolomide, carmustine, cisplatin, carboplatin, topotecan, camptothecin, etoposide, cediranib, erlotinib, gefinitib, glivec, Hydrea, cilengitide, cetuximab, bevacizumab, taxol, vincristine. Advantageously, one or more anticancer agents can be combined with the compound according to the invention.

“Anticancer agent” (also called “antitumour agent” or “antineoplastic agent”) means, in the sense of the present invention, a cytotoxic compound that selectively destroys transformed cells and makes it possible to treat, prevent and/or reduce the severity of a cancer.

The pharmaceutical composition can comprise the compound according to the invention and the anticancer agent in a molar ratio in the range from 10⁴/1 to 1/10⁴, for example from 10³/1 to 1/10³, for example from 10²/1 to 1/10², for example from 10/1 to 1/10.

Other examples of agents with which the compounds according to the present invention can also be combined comprise, without being limited to, any therapeutic agent used for alleviating or treating the side-effects of anticancer treatments (chemoprotection). For example they can be therapeutic agents acting on:
haematological toxicities:
   reduction of the duration of neutropenia and reduction of infectious complications (haematopoietic growth factors, Granocyte®, Neupogen®, Leucomax®), cytoprotective (amifostine);
   correction and prevention of anaemias (recombinant erythropoietin);
gastrointestinal toxicities such as nausea, vomiting, stimulation of the vomiting centre, anticipation (antiemetics), mucositis, stomatitis, transit disorders (diarrhoea, constipation);
renal toxicities such as tubular precipitation and tubular necrosis;
bladder toxicities such as haemorrhagic cystitis;
dermatological toxicities such as alopecia, nail fragility, hyperpigmentation;
neurotoxicities such as disorders of sensitivity, hyporeflexia, constipation, ototoxicity, epileptogenic effects, secretion of antidiuretic hormone, cerebellar disorders;
allergic reactions such as anaphylactic shock;
extravasation (vesicant agents);
chronic toxicities such as:
   myelotoxicity (secondary leukaemias);
   cardiac toxicities (cardiac insufficiency);
   hepatic toxicities (cytolysis) (amifostine);
   neurotoxicities (cortical atrophy);
   pulmonary toxicities (pulmonary fibroses);
   toxicities affecting fertility and gonadal functions (oligo-azoospermine, amenorrhoea).

They may, moreover, be therapeutic agents used in hormonotherapy, for instance hormonotherapy of prostate cancer such as administration of oestrogens (diethylstilbestrol, fosfestrol) or of antiandrogens (flutamide, nilitamide, bicalutamide, cyproterone acetate); hormonotherapy of breast cancer such as administration of progestational agents, administration of Gn-RH analogues, inhibition of biosynthesis of adrenal steroids (formestane, aminoglutethimide, anastrozole, letrozole) or administration of anti-oestrogens (tamoxifen); or hormonotherapy of digestive endocrine tumours such as administration of somatostatin analogues (octreotide, lanreotide).

Other examples of agents with which the compounds according to the present invention can also be combined comprise, but are not limited to: treatments for Alzheimer's disease, such as Aricept® and Excelon®; treatments for Parkinson's disease such as L-DOPA/carbidopa, entacapone, ropinirole, pramipexole, bromocriptine, pergolide, trihexyphenidyl, and amantadine; agents for treating multiple sclerosis (MS), such as interferon beta (for example, Avonex® and Rebif), Copaxone®, and mitoxantrone; treatments for asthma such as albuterol and Singulair®; agents for treating schizophrenia such as Zyprexa®, Risperdal®, Seroquel®, and haloperidol; anti-inflammatory agents such as corticosteroids, anti-TNF, IL-1 RA, azathioprine, cyclophosphamide, and sulfasalazine; immunomodulators and immunosuppressants such as ciclosporin, tacrolimus, rapamycin, mycophenolate, interferons, corticoids, cyclophosphamide, azathioprine, and sulfasalazine; neurotrophic factors such as acetylcholinesterase inhibitors, MAO (monoamine oxidase) inhibitors, interferons, anticonvulsants, ion channel inhibitors, riluzole, and anti-parkinsonian agents; agents for treating cardiovascular diseases such as beta-blockers, angiotensin-converting enzyme inhibitors (ACE inhibitors), diuretics, nitrates, calcium inhibitors and statins; agents for treating liver diseases such as corticosteroids, cholestyramine, interferons and antiviral agents; agents for treating blood disorders such as corticosteroids, antileukaemia agents, and growth factors; and agents for treating immune system disorders such as gamma-globulins.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The pharmaceutically acceptable compounds or compositions according to the present invention can also be used in compositions for coating implantable medical devices, such as prostheses, artificial valves, vascular grafts, stents and catheters. Thus, according to another aspect, the present invention relates to a composition for coating an implantable device comprising a compound of the present invention as described in the present text and a support suitable for coating said implantable device.

According to yet another aspect, the present invention relates to an implantable device coated with a composition comprising a compound of the present invention as described in the present text and a support suitable for coating said implantable device.

Vascular stents, for example, can be used for treating restenosis (narrowing of blood vessel walls again after a wound). However, patients using stents or other implantable devices risk the formation of clots or platelet activation. These undesirable effects can be avoided or attenuated by using a device previously coated with a pharmaceutically acceptable composition comprising a compound according to the present invention. Suitable coatings and the general preparation of coated implantable devices are described for example in U.S. Pat. Nos. 6,099,562; 5,886,026; and 5,304,121. The coatings are generally of biocompatible polymer materials such as a hydrogel polymer, polymethyldisiloxane, polycaprolactone, polyethylene glycol, polylactic acid, vinyl and ethylene acetate, and mixtures thereof. The coatings can optionally be covered in addition with a suitable finishing layer of fluorosilicone, polysaccharides, polyethylene glycol, phospholipids or a combination thereof for endowing the composition with characteristics of controlled release.

### 3/Uses

According to another aspect of the invention, the compounds and compositions described in the present text can be used as medicinal products intended for treating cancer, whatever its nature and its degree of anaplasia.

According to another aspect, the invention also relates to the use of a compound or a pharmaceutically acceptable salt thereof as defined above for manufacturing a pharmaceutical composition intended for treating cancers, whatever their nature and their degree of anaplasia.

According to another aspect, the invention also relates to the use of a compound or a pharmaceutically acceptable salt thereof as defined above for manufacturing a medicinal product intended for treating cancers, whatever their nature and their degree of anaplasia.

According to another aspect, the invention also relates to a compound or a pharmaceutically acceptable salt thereof as defined above for use in the treatment of cancers, whatever their nature and their degree of anaplasia. For example, they can be melanomas, carcinomas, sarcomas, fibrosarcomas, leukaemias, lymphomas, neuroblastomas, medulloblastomas, glioblastomas, astrocytomas, angioblastomas, meningiomas, retinoblastomas, prolactinomas, macrobulimia, leiomyo sarcomas, mesotheliomas, choriocarcinomas, phaeochromocytomas, myelomas, polycythaemias, angio sarcomas, extraskeletal chondrosarcomas, haemangiosarcomas, osteosarcomas, chondrosarcomas, and generally melanomas, carcinomas, sarcomas, fibrosarcomas and leukaemias.

According to another aspect, the present invention relates to a method for treating, preventing or reducing the severity of a cancer, said method comprising the administration of an effective amount of a pharmaceutically acceptable compound or composition according to the invention to an affected subject.

Advantageously, an “effective amount” of the compound or pharmaceutically acceptable composition is that amount effective for exhibiting cytotoxicity towards proliferating and/or quiescent cancer stem cells. The compounds and compositions, according to the method of the present invention, may be administered using any amount and any route of administration effective for treating cancer. The exact amount required will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

The compounds and compositions, according to the method of the present invention, can be administered using any dosage and any route of administration effective for treating, preventing or reducing the severity of cancer. The exact dosage required varies from one subject to another, depending on the species (human or animal), the subject's age and general condition, the severity of the disease, the particular compound, its method of administration, etc.

The compounds of the invention are preferably formulated as a unit dose for ease of administration and uniformity of dose. In the present text, the expression “unit dose” refers to a physically separate unit of the compound suitable for the subject to be treated. It will be understood, however, that decisions relating to the total daily dose of the compounds or compositions according to the present invention are to be made by the treating doctor.

The effective dosage for a particular subject will depend on various factors, including the cancer treated and the severity of the disease; the activity of the specific compound used, the specific composition used, the subject's age, body weight, general state of health, sex and diet, the duration of administration, the route of administration, the rate of excretion of the specific compound used, the duration of the treatment, the medicinal products used in combination or in association with the specific compound used, and other similar factors that are well known in the medical field.

As used herein, the term “subject” denotes an animal, preferably a mammal, preferably a human being at any age.

The pharmaceutically acceptable compounds or compositions according to the present invention can be administered to humans and other animals by the oral, rectal, parenteral, intracisternal, vaginal, intraperitoneal, topical (e.g. as powders, ointments or drops), or buccal route, as a spray by the oral or nasal route, or similar, depending on the severity and the type of cancer treated. For example, the pharmaceutically acceptable compounds or compositions according to the invention can be administered orally or parenterally at doses from 0.01 mg/kg to about 50 mg/kg and preferably from 1 mg/kg to about 25 mg/kg of the subject's body weight per day, one or more times daily, to obtain the desired therapeutic effect.

As examples of cancers that can be treated according to the invention, we may mention pancreatic cancer, oro-pharyngeal cancers, stomach cancer, oesophageal cancer, colon and rectal cancer, brain cancer, notably gliomas, ovarian cancer, liver cancer, kidney cancer, larynx cancer, thyroid cancer, lung cancer, bone cancer, multiple myelomas, mesotheliomas and melanomas, skin cancer, breast cancer, prostate cancer, bladder cancer, uterine cancer, testicular cancer, non-Hodgkin lymphomas, leukaemia, Hodgkin's disease, cancer of the tongue, duodenal cancer, bronchial cancer, pancreatic cancer and soft tissue cancers, as well as the metastatic secondary localizations of the aforementioned cancers such as in the lung, liver or breast.

Advantageously, the compound may be administered in association or in combination with at least one other therapeutic agent, notably at least one anticancer agent different from the compound according to the invention. The reader may refer on this point to the description of combined therapies given above in section 2/“Pharmaceutical compositions”. Not all the variants described in section 2/are reproduced here, but it is to be understood that each of the aforementioned variants is applicable mutatis mutandis to the present embodiment.

Thus, advantageously, the compound according to the invention can be administered in association or in combination with at least one other anticancer agent selected from the group comprising: electrophilic agents (alkylating agents, nitroso-ureas, hydroxyurea, platinum derivatives), intercalating agents, cleaving agents, antimetabolites, enzyme inhibitors (inhibitors of topoisomerases, ribonucleotide reductase, tyrosine kinases, farnesyl transferases), integrin receptor inhibitors, monoclonal antibodies, agents acting on the mitotic spindle, inhibitors of histone deacetylases (HDACs), inhibitors of Akt signalling, Notch signalling, Sonic Hedgehog signalling. They can be for example temozolomide, carmustine, cisplatin, carboplatin, topotecan, camptothecine, etoposide, cediranib, erlotinib, gefinitib, glivec, Hydrea, cilengitidine, cetuximab, bevacizumab, taxol, vincristine. Advantageously, one or more anticancer agents can be combined with the compound according to the invention.

“Associated” or “in association” means that the compound according to the invention and the anticancer agent can be administered simultaneously, separately or spread out over time.

The pharmaceutical compositions according to the invention can be administered by various routes.

As examples of routes of administration, we may mention the oral, rectal, cutaneous, pulmonary, nasal, sublingual route, the parenteral route notably intradermal, subcutaneous, intramuscular, intravenous, intraarterial, intraspinal, intraarticular, intrapleural, intraperitoneal, ocular, inhalation, transdermal, epidural, intrabronchial, intrabursal, intracameral, intracardiac, intracerebral, intracavernous, intracerebroventricular, intracisternal, intragastric, intralesional, intralymphatic, intraosseous, intraspinal, intrathecal, intratracheal, intraduodenal, intratympanic, intraurethral, intrauterine, intravaginal, intravesical, intravitreal, sublabial, rectal, subconjunctival, retrobulbar, intratumoral in particular subconjunctival or retrobulbar, routes.

The pharmaceutical compositions according to the invention can be administered one or more times or with continuous release.

The pharmaceutical composition according to the invention can be administered in one or more daily doses, in particular in 1 to 3 daily doses.

Advantageously, the compound can be administered in an amount in the range from 0.1 to 6 mg per day and per kg.

Currently, no treatment exists that can eradicate cancer stem cells.

Thus, according to one aspect, the invention proposes for the first time a use of bisacodyl and analogues thereof in the treatment of cancers, optionally in association/combination with therapeutic agents and/or existing therapeutic protocols, in particular of cancers having cancer stem cells such as human glioblastomas and melanomas. The use of the compounds according to the present invention therefore constitutes an anticancer treatment having a considerable impact in the medical field since it makes it possible to prevent recurrences of cancers after treatment.

The results presented in the present text, notably in the Examples, in fact very clearly validate the proof of concept, namely that bisacodyl and its analogues are very promising candidates in the treatment of tumours having CSCs capable of going into a quiescent state, notably glioblastomas. Since they target the CSCs, the compounds according to the invention should also make it possible to prevent recurrences of cancers after treatment, which constitutes a major advance in the field of cancer treatment.

According to the present invention, the inventive compounds may be assayed in any of the available assays known in the art for identifying compounds having cytotoxic activity towards proliferating and/or quiescent cancer stem cells. For example, the assay may be cellular or non-cellular, in vivo or in vitro, high- or low-throughput format, etc.

In yet another aspect, there is provided a method of exhibiting cytotoxic activity towards proliferating and/or quiescent cancer stem cells in:

a subject; or
a biological sample;
which method comprises administering to said subject, or contacting said biological sample with:
a composition as described herein; or
a compound as described herein.

Advantageously, the invention provides compounds and compositions as described herein for use in exhibiting cytotoxic activity towards proliferating and/or quiescent cancer stem cells, for example in a subject or a biological sample.

Advantageously, there is provided a method of treating primary mammalian tumor sites and/or metastatic sites in a subject, comprising administering to said subject an effective cytotoxic amount of:

a composition as described herein; or
a compound as described herein.

Advantageously, the invention provides compounds and compositions as described herein for use in treating primary mammalian tumor sites and/or metastatic sites in a subject.

Advantageously, there is provided a method of treating chemo- and/or radio-resistant cancer in a subject, comprising administering to said subject an effective cytotoxic amount of:

a composition as described herein; or
a compound as described herein.

Advantageously, the invention provides compounds and compositions as described herein for use in treating chemo- and/or radio-resistant cancer in a subject.

Advantageously, there is provided a method of preventing or lessening the recurrence of cancer in a subject, comprising administering to said subject an effective cytotoxic amount of:

a composition as described herein; or
a compound as described herein.

Advantageously, the invention provides compounds and compositions as described herein for use in preventing or lessening the recurrence of cancer in a subject.

Advantageously, there is provided a method of treating a cancer in a subject, comprising administering to said subject an effective cytotoxic amount of:

a composition as described herein; or
a compound as described herein;
wherein said cancer is an aggressive cancer. Advantageously, the aggressive cancer may be associated with a greater occurrence of cancer stem cells than other less aggressive cancers.

Advantageously, the invention provides compounds and compositions as described herein for use in treating a cancer in a subject, wherein said cancer is an aggressive cancer. Advantageously, the aggressive cancer may be associated with a greater occurrence of cancer stem cells than other less aggressive cancers.

Advantageously, there is provided a method of preventing cancer in a subject genetically predisposed to cancer, comprising administering to said subject an effective cytotoxic amount of:

a composition as described herein; or
a compound as described herein;
wherein said cancer is associated with cancer stem cells in quiescent state.

Advantageously, the invention provides compounds and compositions as described herein for use in preventing cancer in a subject genetically predisposed to cancer, wherein said cancer is associated with cancer stem cells in quiescent state.

Advantageously, there is provided a screening method for a compound having cytotoxic activity towards cancer stem cells, comprising the steps of: (a) providing cancer stem cells; (b) contacting the cells with a test compound; (c) assessing the cytotoxicity of the test compound to the cells. Advantageously, the cancer stem cells may be proliferating cancer stem cells. Advantageously, the cancer stem cells may be quiescent cancer stem cells. Advantageously, the method may be a high-throughput screening method.

Advantageously, there is provided a screening method for a compound having cytotoxic activity towards quiescent cancer stem cells, comprising the steps of: (a) providing quiescent cancer stem cells; (b) contacting the cells with a test compound; (c) assessing the cytotoxicity of the test compound to the cells. Advantageously, step (a) may comprise providing a culture of cancer stem cells and adjusting the pH of the culture medium to a value<7. For example, the pH may be adjusted to 5.0-6.9, for example 5.5-6.9, for example 5.5-6.7, for example 6.0-6.6. Advantageously, the pH of the culture medium may be adjusted to the desired pH by maintaining cancer stem cells in the same culture medium for a prolonged period of time sufficient for the pH to decrease naturally to an acidic value (the cells consume glucose and release acids, in particular lactic acid and carbonic acid). Advantageously, the pH of the culture medium may be adjusted to the desired pH with a solution of HCl. Advantageously, the pH of the culture medium may be adjusted to the desired pH with a solution of sodium acetate. Advantageously, the pH of the culture medium may be adjusted to the desired pH with a solution of lactic acid. Advantageously, the method may be a high-throughput screening method.

Advantageously, the step of determining the cytotoxicity may comprise measuring the cell ATP-levels.

Advantageously, the step of determining the cytotoxicity may comprise comparing the cell ATP-levels between test-compound-treated and untreated proliferating and/or quiescent cancer stem cells.

The term “measurably inhibit”, as used herein means a measurable change in cytotoxic activity between a sample comprising said composition and proliferating and/or quiescent cancer stem cells and an equivalent sample comprising proliferating and/or quiescent cancer stem cells in the absence of said composition.

Another aspect of the invention relates to exhibiting cytotoxic activity towards proliferating and/or quiescent cancer stem cells in a biological sample or a patient, which method comprises administering to the patient, or contacting said biological sample with any one or more of the compounds described herein or a composition comprising said compound. The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Exhibiting cytotoxic activity towards proliferating and/or quiescent cancer stem cells in a biological sample is useful for a variety of purposes that are known to one of skill in the art. Examples of such purposes include, but are not limited to, blood transfusion, organ-transplantation, biological specimen storage, and biological assays.

### Kits of Parts

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### EQUIVALENTS

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

Other features and advantages will also become apparent to a person skilled in the art on reading the following examples, which are given for purposes of illustration and are non-limiting, referring to the appended drawings.

### BRIEF DESCRIPTION OF THE DRAWINGS

FIG. 1 is a schematic representation of the 96-well plate for the ATP Glo assay where:

[symbol]

represents the positive control with 5×10⁻⁵ M of terfenadine (with 1% of DMSO) [symbol]
represents the negative control with 1% of DMSO
◯ represents the other wells containing a test molecule at a given concentration (with 1% of DMSO)

FIG. 2 is a graph showing the effect of bisacodyl (at 50 μM) on TG1 cancer stem cells, quiescent (Q) and proliferating (P) isolated from a patient, expressed as a percentage of survival relative to cells in the same conditions but in the absence of bisacodyl (SC (%). For screening I (Cl), the test was conducted on a sample. Screening II (C2) shows the result obtained in an experiment conducted independently (n=2).

FIG. 3a is a graph showing the effect of compound 1 (bisacodyl) on survival of TG1 cancer stem cells (SC (%)), quiescent (Q) and proliferating (P) isolated from a glioblastoma of a patient 1 as a function of the concentration of compound 1 in μM, expressed as a percentage relative to cells in the same conditions but in the absence of compound 1.

FIG. 3b is a graph showing the effect of compound 1 (bisacodyl) on survival of cancer stem OB1 cells (SC (%)), quiescent (Q) and proliferating (P) isolated from a glioblastoma of a patient 2 as a function of the concentration of compound 1 in μM, expressed as a percentage relative to cells in the same conditions but in the absence of compound 1.

FIG. 3c is a graph showing the effect of compound 1 (bisacodyl) on survival of cancer stem TG16 cells (SC (%)), quiescent (Q) and proliferating (P) isolated from a glioblastoma of a patient 3 as a function of the concentration of compound 1 in μM, expressed as a percentage relative to cells in the same conditions but in the absence of compound 1.

FIG. 3d is a graph showing the effect of compound 1 (bisacodyl) on survival of U-87 MG cells (SC (%)), as a function of the concentration of compound 1 in μM, expressed as a percentage relative to cells in the same conditions but in the absence of compound 1.

FIG. 3e is a graph showing the effect of compound 1 (bisacodyl) on survival of fetal neural stem f-NSC cells (SC (%)), as a function of the concentration of compound 1 in μM, expressed as a percentage relative to cells in the same conditions but in the absence of compound 1.

FIG. 3f is a graph showing the effect of compound 1 (bisacodyl) on survival of HA cells of human astrocytes (SC (%)), as a function of the concentration of compound 1 in μM, expressed as a percentage relative to cells in the same conditions but in the absence of compound 1.

FIG. 3g is a graph showing the effect of compound 1 (bisacodyl) on survival of cells HEK293 (SC (%)), as a function of the concentration of compound 1 in μM, expressed as a percentage relative to cells in the same conditions but in the absence of compound 1.

FIG. 4a is a graph showing the effect of compound 2 (DDPM) on survival of TG1 cancer stem cells (SC (%)), quiescent (Q) and proliferating (P) isolated from a glioblastoma of a patient 1 as a function of the concentration of compound 2 in μM, expressed as a percentage relative to cells in the same conditions but in the absence of compound 2.

FIG. 4b is a graph showing the effect of compound 2 (DDPM) on survival of cancer stem OB1 cells (SC (%)), quiescent (Q) and proliferating (P) isolated from a glioblastoma of a patient 2 as a function of the concentration of compound 2 in μM, expressed as a percentage relative to cells in the same conditions but in the absence of compound 2.

FIG. 4c is a graph showing the effect of compound 2 (DDPM) on survival of U-87 MG cells (SC (%)), as a function of the concentration of compound 2 in μM, expressed as a percentage relative to cells in the same conditions but in the absence of compound 2.

FIG. 4d is a graph showing the effect of compound 2 (DDPM) on survival of fetal neural stem f-NSC cells (SC (%)), as a function of the concentration of compound 2 in μM, expressed as a percentage relative to cells in the same conditions but in the absence of compound 2.

FIG. 4e is a graph showing the effect of compound 2 (DDPM) on survival of HA cells of human astrocytes (SC (%)), as a function of the concentration of compound 2 in μM, expressed as a percentage relative to cells in the same conditions but in the absence of compound 2.

FIG. 5a is a graph showing the effect of compound 3 on survival of TG1 cancer stem cells (SC (%)), quiescent (Q) and proliferating (P) isolated from a glioblastoma of a patient 1 as a function of the concentration of compound 3 in μM, expressed as a percentage relative to cells in the same conditions but in the absence of compound 3.

FIG. 5b is a graph showing the effect of compound 3 on survival of fetal neural stem f-NSC cells (SC (%)), as a function of the concentration of compound 3 in μM, expressed as a percentage relative to cells in the same conditions but in the absence of compound 3.

FIG. 6a is a graph showing the effect of compound 5 on survival of TG1 cancer stem cells (SC (%)), quiescent (Q) and proliferating (P) isolated from a glioblastoma of a patient 1 as a function of the concentration of compound 5 in μM, expressed as a percentage relative to cells in the same conditions but in the absence of compound 5.

FIG. 6b is a graph showing the effect of compound 5 on survival of fetal neural stem f-NSC cells (SC (%)), as a function of the concentration of compound 5 in μM, expressed as a percentage relative to cells in the same conditions but in the absence of compound 5.

FIG. 7a is a graph showing the effect of compound 6 on survival of TG1 cancer stem cells (SC (%)), quiescent (Q) and proliferating (P) isolated from a glioblastoma of a patient 1 as a function of the concentration of compound 6 in μM, expressed as a percentage relative to cells in the same conditions but in the absence of compound 6.

FIG. 7b is a graph showing the effect of compound 6 on survival of fetal neural stem f-NSC cells (SC (%)), as a function of the concentration of compound 6 in μM, expressed as a percentage relative to cells in the same conditions but in the absence of compound 6.

FIG. 8a is a graph showing the effect of compound 7 on survival of TG1 cancer stem cells (SC (%)), quiescent (Q) and proliferating (P) isolated from a glioblastoma of a patient 1 as a function of the concentration of compound 7 in μM, expressed as a percentage relative to cells in the same conditions but in the absence of compound 7.

FIG. 8b is a graph showing the effect of compound 7 on survival of fetal neural stem f-NSC cells (SC (%)), as a function of the concentration of compound 7 in μM, expressed as a percentage relative to cells in the same conditions but in the absence of compound 7.

FIG. 9a is a graph showing the effect of compound 9 on survival of TG1 cancer stem cells (SC (%)), quiescent (Q) and proliferating (P) isolated from a glioblastoma of a patient 1 as a function of the concentration of compound 9 in μM, expressed as a percentage relative to cells in the same conditions but in the absence of compound 9.

FIG. 9b is a graph showing the effect of compound 9 on survival of fetal neural stem f-NSC cells (SC (%)), as a function of the concentration of compound 9 in μM, expressed as a percentage relative to cells in the same conditions but in the absence of compound 9.

FIG. 10. Non-proliferating viable quiescent glioblastoma stem cells can be obtained in vitro by maintaining cells in culture without medium renewal. (A) In the absence of serum, proliferating glioblastoma stem cells grow as neurospheres in culture. (B) Non-proliferating quiescent glioblastoma stem cells are generated in vitro by leaving cells in culture without medium change for 9-16 days. These cells are morphologically similar to proliferating cells although neurospheres could be more easily dissociated Also when considering the whole culture plate, the spheres are less numerous. Several bigger neurospheres were also observed. Scale bars, 100 μm. (C-D) Proliferation and survival curves of TG1 glioblastoma stem cells grown in culture for 1-16 days without growth-medium renewal. Cells going through the S phase were identified through EdU (5-ethynyl-2′-deoxyuridine) incorporation and cell viability was estimated by the percentage of unstained cells in the presence of 7-AAD (7-aminoactinomycin D). Bars denote the standard error (n=3).

FIG. 11. The Prestwick chemical library was screened using the ATP-Glo cell based assay. (A) Schematic representation of the assay design and protocol. (B) Results of the primary screen are represented as histograms of the ATP level (expressed as a percentage of control) obtained for the compounds of the Prestwick chemical library screened against proliferating (black bars) and quiescent (open bars) TG1 glioblastoma stem cells. Molecules producing a ATP level signal that exceeded 200% are not shown. An enlargement of the zone of results for compounds with a ATP level of less than 65% is also given. A compound was considered as a hit if it reduced the ATP level to less than 5% (compounds on the left of the black dotted line) or if it produced a luminescent signal, reflecting ATP levels in a well that was lower than the mean signal of negative control wells minus 5 times the standard deviation from this value. The number of molecules selected according to the second criteria is indicated on the top of each bar. (C) 86 compounds reducing the ATP level and 16 molecules increasing the ATP level in the corresponding well were further tested in a secondary screen (5 μM and 50 μM). Results of the secondary screen (50 μM) are represented as in B. Hit selection for quiescent cells was as for the primary screen whereas, for proliferating cells, compounds were validated if they produced an ATP signal that was lower than the mean signal of negative controls minus 3 times the standard deviation from this value.

FIG. 12. Identification and validation of potent glioblastoma stem cell specific compounds. (A and B) Chemical structures and dose-response curves of selected compounds (Suloctidil (left), Zuclopenthixol HCl (middle) and bisacodyl (right)) with representative activity profiles on proliferating (▪) and quiescent (▴) TG1 cells. The fitted sigmoidal logistic curve (see Materials and methods section) to ATP-Glo cell survival assay readings is shown on each plot. Values represent the mean and standard deviation of three independent experiments (n=3). (B) Dose-response curves of Suloctidil (left), Zuclopenthixol HCl (middle) and bisacodyl (right) on TG1 glioblastoma stem cells (▪ for proliferating cells, ▴ for quiescent cells), human primary astrocytes (⋄) and human fetal neural stem cells (♦). Curves are fitted as in (A). Plotted values are from three independent experiments (n=3). (C) Graphical presentation of the activity of the three selected compounds (expressed as 1/Efficient concentration leading to 50% change in activity and indicated as 1/EC50) on: proliferating (P) and quiescent (Q) glioblastoma stem like cells derived from three patients (TG1, TG16 and OB1), human primary astrocytes (HA cells), human fetal neural stem cells (f-NSC), a human embryonic kidney cell line (HEK 293) and the U-87 MG glioblastoma cell line. EC50 values for each compound and cell type were the mean of EC50 values from fitted dose-response curves to ATP-Glo cell viability assay readings from three independent experiments (n=3). Given that the maximum effect of bisacodyl on HA cells is a 30% reduction of ATP levels that is not due to cell death (see FIG. 13B and observed using trypan blue staining (data not shown)), the corresponding EC50 value was not taken into account. (TG1 cells are also referred to as “TG01 cells”)

FIG. 13. Effect of the culture medium on the activity of Bisacodyl and its metabolite DDPM on glioblastoma stem cells. (A) Stability of Bisacodyl in proliferating (Δ) and quiescent (□) glioblastoma stem cell culture medium. Similar experiments were performed for DDPM in proliferating (▴) and quiescent (▪) conditioned culture medium. (B-C) Plots of dose-response curves of DDPM on the viability of proliferating (B) or quiescent (C) glioblastoma stem cells. The 24-hour treatment with this compound was performed either in freshly prepared (▴) or in quiescent conditioned culture medium (▪). Plotted values and bars are, respectively, the mean and standard deviation of cell viability readings obtained in two independent experiments (n=2).

FIG. 14. The cytotoxic activity of DDPM is pH-dependent (A) pH measurements of the culture medium of glioblastoma stem cells maintained without growth factor renewal for 1-16 days. The values plotted represent the mean and standard deviation from 4 independent experiments (n=4) (B) Histograms of DDPM cytotoxic activity (ATP-Glo cell survival readings) on proliferating (black bars) and quiescent (grey bars) glioblastoma stem cells. Cells were treated with the selected compound (10 μM) for 24 h in freshly prepared culture medium at pH values varying from 7.4 to 6. In this experiment, pH was adjusted with a 1M HCl solution. Given results and standard deviations are from four independent experiments (n=4) (C) Histograms of DDPM cytotoxic activity (ATP-Glo cell survival readings) on proliferating (black bars) and quiescent (grey bars) glioblastoma stem cells. Cells were treated with the selected compound (100 μM) for 24 h in freshly prepared culture medium at pH values varying from 7.0 to 6.4. In this experiment, pH was adjusted with a 0.1M sodium acetate buffer at pH=4 solution. Given results and standard deviations are from 1 experiment (n=1) (D) Dose-response curves of DDPM were performed on the U-87 MG glioblastoma cell line in its culture medium at pH=7.4 (♦), at pH=6.2 adjusted with a 1M HCl solution (⋄) and at pH=6.2 adjusted with a 0.1M sodium acetate buffer (pH=4) solution (Δ). Results are from at least two independent experiments (n=2) (E) Dose-response curves of DDPM were performed on human primary astrocytes (HA cells) in their culture medium at pH=7.4 (♦), at pH=6.2 adjusted with a 1M HCl solution (⋄) and at pH=6.2 adjusted with a 0.1M sodium acetate buffer (pH=4) solution (Δ). Results are from at least two independent experiments (n=2).

FIG. 15. pH-dependent stimulation of apoptosis by DDPM in glioblastoma stem cells (A-B) Histograms of fluorescent signal (485-510 nm) intensities reflecting caspase 3/7 activity following a 24 h treatment of proliferating (A) or quiescent (B) glioblastoma stem cells with increasing concentrations of DDPM. Compound treatment was performed in freshly prepared culture medium at pH=7.3 (black bars) or at pH=6.4 (open bars). Staurosporine (SSP) (1 μM) and culture medium alone (medium) were used as positive and negative controls, respectively. Results are from 2 independent experiments. Error bars denote the standard error from mean values.

FIG. 16. (A) Effect of a 24 h exposure of TG1 glioblastoma cancer stem cells to TMZ (▪) or to DPPM in the absence (♦) and presence () of 60 μM of TMZ in NS34 culture medium at pH 7.35; (B) Effect of a 72 h exposure of TG1 glioblastoma cancer stem cells to TMZ (▪) or to DPPM in the absence (♦) and presence () of 60 μM of TMZ in NS34 culture medium at pH 7.35; (C) Effect of a 24 h exposure of TG1 glioblastoma cancer stem cells to TMZ (▪) or to DPPM in the absence (♦) and presence () of 60 μM of TMZ in NS34 culture medium at pH 6.2; and (D) Effect of a 72 h exposure of TG1 glioblastoma cancer stem cells to TMZ (▪) or to DPPM in the absence (♦) and presence () of 60 μM of TMZ in NS34 culture medium at pH 6.28.

### EXEMPLIFICATION

The compounds of this invention and their preparation can be understood further by the examples that illustrate some of the processes by which these compounds are prepared or used. It will be appreciated, however, that these examples do not limit the invention. Variations of the invention, now known or further developed, are considered to fall within the scope of the present invention as described herein and as hereinafter claimed.

The following abbreviations are used in the examples.
7-AAD=7-amino actinomycin D
EtOAc=ethyl acetate
BSA=bovine serum albumin
CaCl₂=calcium chloride
CaO=calcium oxide
TLC=preparative thin-layer chromatography
CH₂Cl₂=dichloromethane
DMEM=Dulbecco's Modified Eagle's Medium (culture medium)
DMAP=4-dimethylaminopyridine
DMF=dimethylformamide
DMSO=dimethylsulphoxide
eq=equivalent
EdU=5-ethynyl-2′-deoxyuridine
Et₂O=diethyl ether
EtOH=ethanol
KOH=potassium hydroxide
HCl=hydrochloric acid
LiOH=lithium hydroxide
mL=millilitre
Na=sodium
NaH=sodium hydride
Na₂CO₃=sodium carbonate
NaHCO₃=sodium bicarbonate
P₂O₅=phosphorus pentoxide
PBS=phosphate-buffered saline
THF=tetrahydrofuran
TMS=tetramethylsilane

All the chemical compounds used are obtained from Aldrich, Fluka or Acros and are of standard quality.

The solvents are distilled before use.

General Information

The reactions were carried out with magnetic stirring and under an argon atmosphere in flame-dried glassware, unless stated otherwise.

CH₂Cl₂ was dried over CaCl₂ and distilled on P₂O₅.

Tetrahydrofuran (THF) (Aldrich, 34865) was predried on KOH and distilled on Na/benzophenone.

Dimethylformamide (DMF) (Aldrich, 270547) was stirred together with KOH (Aldrich, P1767) and distilled on CaO (Aldrich, 208159) and stored on molecular sieve (Aldrich, 208590, 4 Angstrom, 4-8 mesh beads).

Preparative thin-layer chromatography (TLC) was carried out on silica gel plates (Merck, 60F-254).

Flash chromatography was performed on a column (Merck, 60F-254) with 40 to 63 μm of SiO₂.

The ¹H NMR and ¹⁹F NMR analyses (Brucker UltraShield Plus) were carried out at room temperature (23° C.) at 300 MHz using the residues of the deuterated solvent for calibration as internal standard. The chemical shifts were recorded in parts per million (δ, ppm) in CDCl₃, CD₃OD, or (CD₃)₂CO. The multiplicities are given as the integration and coupling constant (J) in hertz (Hz) with s=singlet, brs=broad singlet, d=doublet, t=triplet, q=quadruplet, o=octuplet and m=multiplet.

### Synthesis of the Compounds

### A. General Methods of Synthesis of the Compounds According to the Invention

### Method A: Reactions Catalysed by Triflic Acid

### Method A.

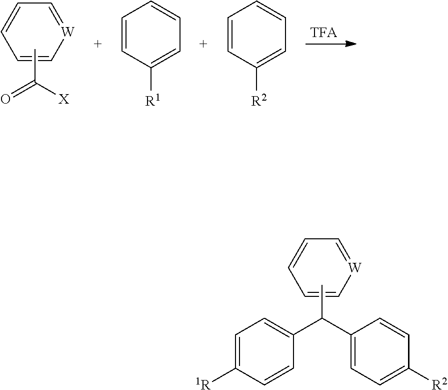

3 mL of triflic acid (TFA) was added to 1 mmol of pyridinecarboxaldehyde (W═N, X═H) and 1.0 mL of aryl compounds (Ph-R¹ and Ph-R²).

After the starting aldehyde had been consumed, the mixture was poured onto ice.

The solution was neutralized with aqueous NaOH solution (15% M), and the products were extracted with CHCl₃.

The organic phase was then washed with water, brine, and then dried on cotton.

Vacuum concentration was carried out to obtain the crude products, which were then purified by recrystallization or by column chromatography.

### Method B: Reactions of Organic Magnesium Compounds (Grignard)

### Method B.

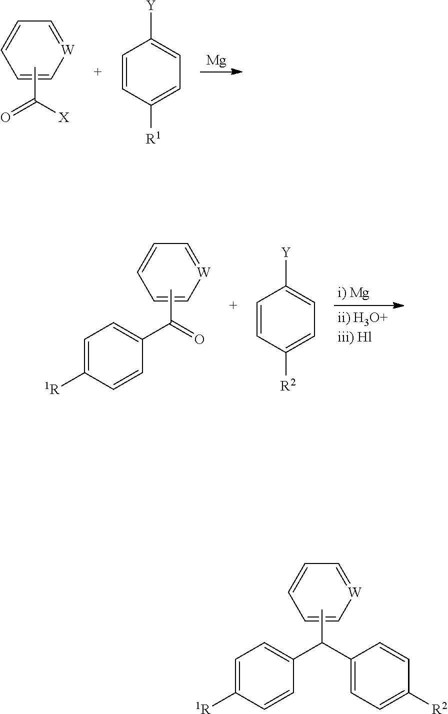

### Symmetric Compounds

With stirring, aryl bromide (Y═Br) in Et₂O (1 M) was added to a suspension of metallic magnesium in the same solvent.

After the end of addition, a solution of the pyridine carboxyester (X═OEt, W═N) in Et₂O (0.5 eq) was added dropwise to the reaction mixture. After hydrolysis, the alcohol is reduced by HI and the final compound purified by chromatography.

### Dissymmetric Compounds

With stirring, aryl-R¹ bromide in Et₂O (0.2 M) was added to a suspension of metallic magnesium in the same solvent.

After the end of addition, a solution of pyridine carboxyester (X═OEt, W═N) in Et₂O (1 eq) was added dropwise to the reaction mixture.

The mixture thus obtained was added slowly to a suspension of aryl-R² bromide and metallic magnesium in Et₂O (1 eq).

The unreacted magnesium was treated (“quenched”) with water, at a temperature of 0° C.

The organic extracts were then washed with water and dried on cotton.

Vacuum concentration was carried out to obtain the crude products. The alcohol was then reduced by HI and the final compound was then purified by recrystallization or by column chromatography.

### Method C: Selective O-Alkylation Reactions

### Method C.

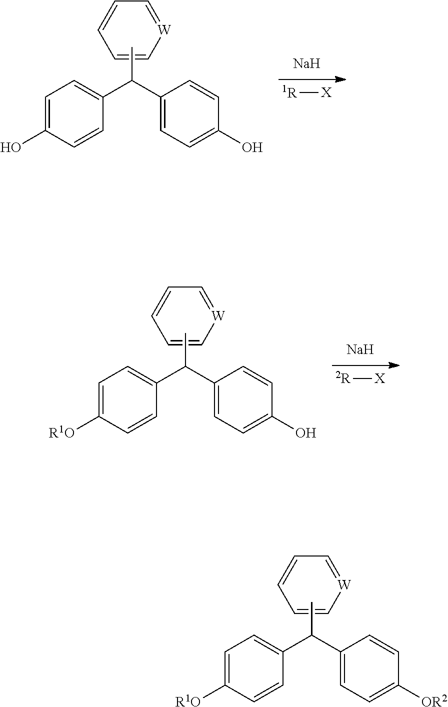

The bisphenol derivative, prepared by method A or B, was dissolved in DMF (0.2 M).

NaH (1 eq) was then added at 0° C. and the resultant suspension was stirred for 5 min.

Alkyl-R¹ bromide (1 eq) was added and the mixture was stirred further at room temperature (22° C.) until the alkyl bromide had been consumed completely.

After cooling to 0° C., NaH (1 eq) was added and the resultant suspension was stirred at room temperature for 10 min before adding alkyl-R² bromide (1 eq).

The resultant solution was stirred until exhaustion of the starting triarylmethane derivative.

Then 35 mL of a 15% aqueous solution of NH₄Cl was added, followed by 60 mL of CH₂Cl₂.

The organic phase was washed with 10 mL of a 10% aqueous solution of LiCl, water (30 mL) and dried on cotton. The crude concentrated product was purified by column chromatography.

### Method D: Stepwise Deprotection and O-Alkylation Reactions

### Method D.

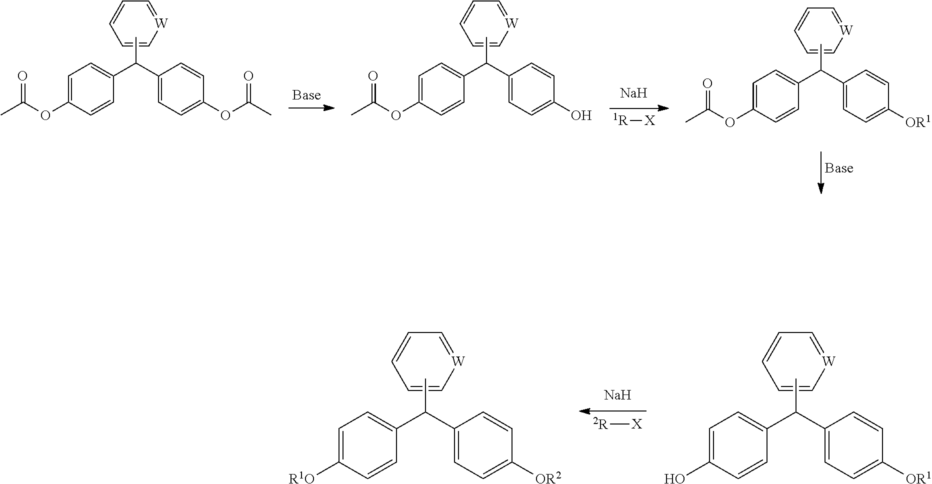

The symmetric esters result from the acetylation of the bisphenol prepared using method A or B.

As a general rule, acetylation was carried out at room temperature (22° C.) by reaction of bisphenol (0.2 M) and acetic anhydride in CH₂Cl₂ (0.5 M) in the presence of a catalytic amount of DMAP (4-dimethylaminopyridine, 20 mol. %).

Selective hydrolysis is obtained using LiOH (1 eq; 0.2 M) as base in a water/1,2-diethoxyethane mixture (20/5).

For the O-alkylation reactions, see method C.

### Example 1

### Specific Examples of Synthesis of the Compounds According to the Invention

### Example 1a

Bisacodyl CAS: 603-50-9 (also denoted Compound 1 (or GSC-001) in the present text) can be bought from Sigma (ref.: B1390). It was synthesized from the corresponding bisphenol (example 1b) prepared by method A or B. This compound satisfies the criteria of purity of said commercial product and of effective and selective activity on cancer stem cells.

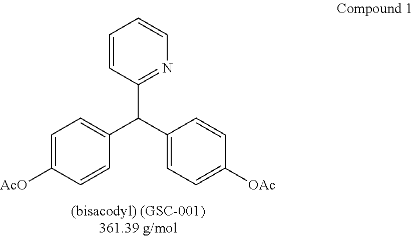 [SMILES: CC(=O)Oc1ccc(C(c2ccc(OC(C)=O)cc2)c2ccccn2)cc1]

### Example 1b

### 4,4′-(Pyridin-2-ylmethylene)diphenol or DDPM (Compound 2 (GSC-002))

4,4′-Dihydroxy-diphenyl-(2-pyridyl)-methane (DDPM, CASE 603-41-8) (also denoted compound 2 (or GSC-002)) was either obtained from Sigma (ref.: 5517631-1EA) or was synthesized by method A or B or prepared from compound 1 (GSC-001) as described below.

The four molecules give the same results.

10 g of (pyridin-2-ylmethylene)bis(4,1-phenylene)diacetate (27.67 mmol; 1 eq) was treated by adding 110 mL of a solution of KOH containing 10% of EtOH.

The mixture was stirred overnight at room temperature (22° C.).

The solvent was evaporated under vacuum to give a solid, which was re-dissolved in 100 mL of EtOAc. The organic phase was acidified with HCl (1 mol/l) until the pH was between 1 and 2, then alkalized with a saturated solution of Na₂CO₃ until the pH was between 8 and 9, and washed 3 times with 10 mL of brine (saturated NaCl solution), and 3 more times with 15 mL of water. The organic phase was then dried on cotton to give a white solid, which was washed with cold CH₂Cl₂ (50 mL) and/or purified by flash chromatography (CH₂Cl₂/EtOAc: 60/40) to give 7.65 g of compound 2 (GSC-002) in the form of a white crystalline solid.

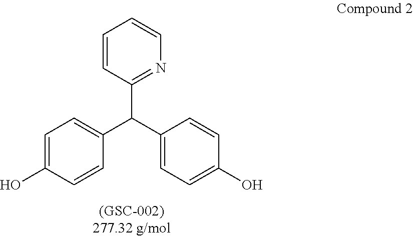 [SMILES: Oc1ccc(C(c2ccc(O)cc2)c2ccccn2)cc1]

### Characteristics of Compound 2 (GSC-002)

All the analysis results correspond to those of a commercial source.

### Example 1c (Method D)

### 4-((4-Hydroxyphenyl)(pyridin-2-yl)methyl)phenyl acetate (Compound 3 (GSC-006))

1 g of (pyridin-2-ylmethylene)bis(4,1-phenylene)diacetate (2.76 mmol; 1 eq) was dissolved in 30 mL of 1,2-dimethoxyethane and treated by adding a solution of 86 mg of LiOH (3.59 mmol; 1.3 eq) in 15 mL of water.

The mixture was stirred for one hour at room temperature (22° C.).

The solvents were evaporated under vacuum to give a solid, which was re-dissolved in 35 mL of EtOAc. The organic phase was washed once with 15 mL of brine, and 3 times with 20 mL of water and then dried on cotton. Final purification was carried out by flash chromatography (CH₂Cl₂/EtOAc: 100/0 to 80/20) to give, at a yield of 46%, 405 mg of racemic compound 3 (GSC-006) in the form of a white crystalline solid.

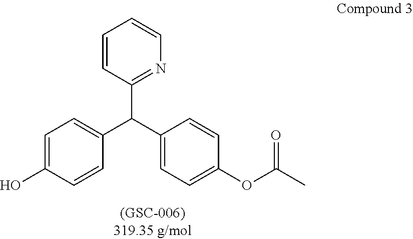 [SMILES: CC(=O)Oc1ccc(C(c2ccc(O)cc2)c2ccccn2)cc1]

### Characteristics of Compound 3 (GSC-006)

¹H NMR (300 MHz, CDCl₃) δ 2.29 (s, 3H), 5.31 (s, 1H), 6.51 (d, 2H, J=8.4), 6.81 (d, 2H, J=8.4), 7.01 (d, 2H, J=8.1), 7.07 (d, 1H, J=7.8), 7.16 (d, 2H, J=8.4), 7.21 (d, 1H, J=6.6), 7.66 (t, 1H, J=7.7), 7.80 (brs, 1H), 8.59 (d, 1H, J=4.8).

### Example 1d

### 2-(bis(4-Hydroxyphenyl)methyl)-1-methylpyridin-1-ium (Compound 4 (GSC-010))

100 mg of 4,4′-(pyridin-2-ylmethylene)diphenol (360.6 μmol; 1 eq) was dissolved in 5 mL of acetone. 1 mL of CH₃I was added to the solution and the mixture was left to stand for 2 hours at room temperature (22° C.).

The solvent was then evaporated under vacuum to give an orange-coloured oil. The oil was washed with 10 mL of pentane and 20 mL of Et₂O to give, at a yield of 83%, 150 mg of compound 4 (GSC-010) in the form of a pale orange crystalline salt.

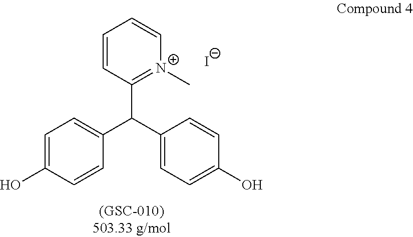 [SMILES: C[n+]1ccccc1C(c1ccc(O)cc1)c1ccc(O)cc1 ; [I-]]

### Characteristics of Compound 4 (GSC-010)

¹H NMR (300 MHz, CD₃OD) (s, 1H) δ 4.26 (s, 3H), 6.04, 6.80-6.84 (m, 2H), 6.82 (d, 2H, J=8.7), 6.92-6.97 (m, 2H), 6.95 (d, 2H, J=8.4), 7.55 (dd, 1H, J=8.25, 1.4), 7.93 (td, 1H, J=7.7, 1.5), 8.44 (td, 1H, J=9.1, 1.3), 8.88 (d, 1H, J=5.7).

### Example 1e

### (Pyridin-2-ylmethylene)bis(4,1-phenylene)bis(2,2,2-trifluoroacetate) (Compound 5 (GSC-012))

50 mg of 4,4′-(pyridin-2-ylmethylene)diphenol (180.3 μmol; 1 eq) was dissolved in 9 mL of CH₂Cl₂ and treated by adding a solution of 45 mg of DMAP (368.3 μmol; 1.9 eq) and 50 μl of trifluoroacetic anhydride (360.6 μmol; 1.9 eq).

The mixture was stirred for one hour at room temperature (22° C.).

Evaporation of the reaction mixture under vacuum gave an orange-coloured oil, which was re-dissolved in 25 mL of CH₂Cl₂.

The organic phase was washed 3 times with 25 mL of water and dried on cotton, to give, at a quantitative yield above 99%, 84 mg of fluorinated compound 5 (GSC-012).

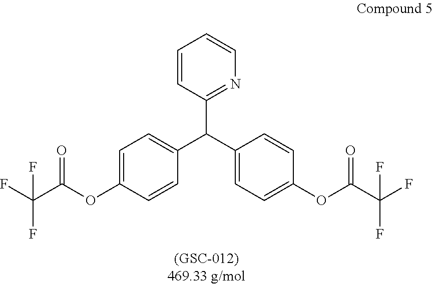 [SMILES: O=C(Oc1ccc(C(c2ccc(OC(=O)C(F)(F)F)cc2)c2ccccn2)cc1)C(F)(F)F]

### Characteristics of Compound 5 (GSC-012)

¹H NMR (300 MHz, acetone-d6) δ 5.85 (s, 1H), 6.79 (d, 4H, J=8.7), 7.05 (d, 4H, J=8.1), 7.49 (d, 1H, J=8.1), 7.61 (td, 1H, J=6.3, 0.6), 8.15 (td, 1H, J=7.7, 1.3), 8.72 (d, 1H, J=4.2).

¹⁹F NMR (300 MHz, acetone-d6) δ −74.8 (s, 6F).

### Example 1f (Method C)

### 4-((4-Methoxyphenyl)(pyridin-2-yl)methyl)phenol (Compound 6 (GSC-018))

100 mg of 4,4′-(pyridin-2-ylmethylene)diphenol (360.6 μmol; 1 eq) was dissolved in 25 mL of DMF.

NaH (60% in mineral oil; 16 mg; 400.1 μmol; 1 eq) was added in small fractions to the reaction mixture at 0° C.

After stirring for 7 minutes at 0° C., 22.5 μl of CH₃I (360.6 μmol; 1 eq) was added.

The resultant violet solution was stirred for a further 3 hours at room temperature (22° C.) before being treated (“quenched”) with 15 mL of water.

30 mL of CH₂Cl₂ was added to the solution.

The organic phase was washed with 20 mL of a saturated solution of NaHCO₃, as well as with an aqueous solution composed of 1 g of LiCl in 20 mL of water, and finally 3 times with 20 mL of water, and finally dried on cotton.

The final concentrated product obtained was purified by flash chromatography (CH₂Cl₂/EtOAc: 100/0 to 85/15) to give, at a yield of 76%, 80 mg of racemic compound 6 (GSC-018) in the form of an orange-coloured oil.

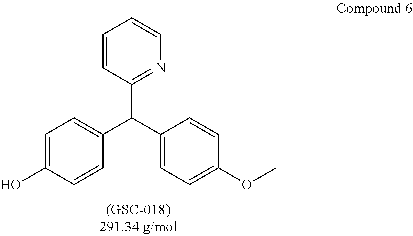 [SMILES: COc1ccc(C(c2ccc(O)cc2)c2ccccn2)cc1]

### Characteristics of Compound 6 (GSC-018)

¹H NMR (300 MHz, CDCl₃) δ 3.79 (s, 3H), 5.61 (s, 1H), 6.58 (dd, 2H, J=8.4, 2.3), 6.81-6.90 (m, 5H), 7.04-7.11 (m, 3H), 7.17 (dd, 1H, J=5.1, 0.9), 7.64 (td, 1H, J=7.7, 1.7), 8.59 (dd, 1H, J=4.8, 0.9).

### Example 1g (Method C or D)

### 4-((4-Methoxyphenyl)(pyridin-2-yl)methyl)phenyl acetate (Compound 7 (GSC-019))

Route A.

75 mg of 4-((4-hydroxyphenyl)(pyridin-2-yl)methyl)phenyl acetate (235.6 μmol; 1 eq) was dissolved in 5 mL of DMF.

NaH (60% in mineral oil; 10.1 mg; 251.9 μmol; 1.1 eq) was added in small fractions to the reaction mixture at 0° C.

After 5 minutes of stirring at this temperature, the green mixture was left to stand for 15 minutes at room temperature (22° C.), then 20 μl of CH₃I (325 μmol; 1.4 eq) was added dropwise.

The resultant blue solution was treated (“quenched”) with 6 mL of a saturated aqueous solution of NH₄Cl.

The yellow solution was extracted with 20 mL of CH₂Cl₂.

The organic phase was washed twice with an aqueous solution composed of 1 g of LiCl in 20 mL of water and 3 times with 25 mL of water, and finally dried on cotton.

The final concentrated product obtained was purified by flash chromatography (CH₂Cl₂/EtOAc: 100/0 to 90/10) to give, at a yield of 68%, 53 mg of chiral compound 7 (GSC-019) in the form of a pale orange-coloured oil.

Route B.

20 mg of compound 6 (GSC-018) (68.65 μmol; 1 eq) was dissolved in 3 mL of CH₂Cl₂ and treated by adding 16 mg of DMAP (131 μmol; 1.9 eq) and 12 μl of acetic anhydride (131 μmol; 1.9 eq).

The mixture was stirred for 2 hours at room temperature (22° C.).

15 mL of CH₂Cl₂ was added.

The organic phase was washed twice with 10 mL of water and dried on cotton to give, at a yield of 99%, 22 mg of compound 7 (GSC-019).

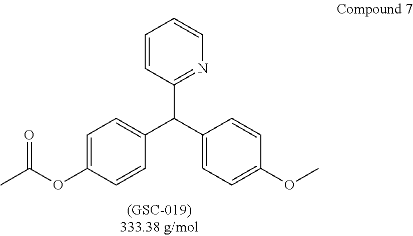 [SMILES: COc1ccc(C(c2ccc(OC(C)=O)cc2)c2ccccn2)cc1]

### Characteristics of Compound 7 (GSC-019)

¹H NMR (300 MHz, CDCl₃) δ 2.28 (s, 3H), 3.79 (s, 3H), 5.63 (s, 1H), 6.84 (d, 2H, J=8.4), 7.02 (d, 2H, J=8.4), 7.1 (d, 2H, J=8.4), 7.17 (d, 2H, J=7.8), 7.18 (d, 2H, J=7.8), 7.61 (t, 1H, J=7.7), 8.60 (dd, 1H, J=5.0, 1.3).

### Example 1h (Method D)

### 4-((4-(Allyloxy)phenyl)(pyridin-2-yl)methyl)phenyl acetate (Compound 8 (GSC-027))

100 mg of 4-((4-hydroxyphenyl)(pyridin-2-yl)methyl)phenyl acetate (314.1 μmol; 1 eq) was dissolved in 5 mL of DMF.

NaH (60% in mineral oil; 13.8 mg; 345.5 μmol; 1.1 eq) was added in small fractions to the reaction mixture at 0° C.

The mixture obtained was left to stand for 15 minutes at room temperature (22° C.) before adding 60 μl of allyl bromide (693.3 μmol; 2.2 eq).

The solution was stirred again for 30 minutes and treated (“quenched”) with 10 mL of water.

25 mL of CH₂Cl₂ was added.

The organic phase was washed twice with an aqueous solution composed of 1 g of LiCl in 25 mL of water and 3 times with 25 mL of water, and finally dried on cotton.

The final concentrated product obtained was purified by flash chromatography (CH₂Cl₂/EtOAc: 100/0 to 96/4) to give, at a yield of 63%, 70 mg of racemic compound 8 (GSC-027) in the form of an orange-coloured oil.

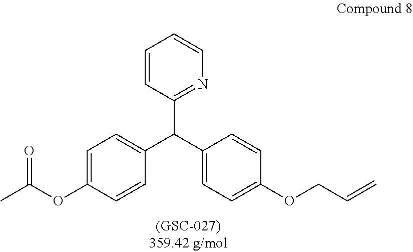 [SMILES: C=CCOc1ccc(C(c2ccc(OC(C)=O)cc2)c2ccccn2)cc1]

### Characteristics of Compound 8 (GSC-027)

¹H NMR (300 MHz, CDCl₃) δ 2.88 (s, 3H), 4.52 (d, 2H, J=5.1), 5.28 (dd, 1H, J=10.7, 1.1), 5.41 (dd, 1H, 17.2, 1.1), 5.63 (s, 1H), 5.98-6.12 (o, 1H), 6.86 (d, 2H, J=8.7), 7.02 (d, 2H, J=8.4), 7.06-7.14 (m, 4H), 7.17 (d, 2H, J=8.4), 7.61 (td, 1H, J=7.7, 1.4), 8.60 (dd, 1H, J=4.5, 0.8).

### Example 1i (Method C)

### 4-((4-(Prop-2-yn-1-yloxy)phenyl)(pyridin-2-yl)methyl)phenol (Compound 9 (GSC-028))

100 mg of 4,4′-(pyridin-2-ylmethylene)diphenol (360.6 μmol; 1 eq) was dissolved in 10 mL of DMF.

NaH (60% in mineral oil; 11 mg; 274.8 μmol; 1.2 eq) was added in small fractions to the reaction mixture at 0° C.

The mixture obtained was stirred for more than 15 minutes until the solution reached room temperature (22° C.).

Next, a solution of 80% of propargyl bromide (39 μL; 360.6 μmol; 1 eq) in toluene was added via a syringe and the contents were stirred for 2 hours at ambient temperature of 22° C.

The reaction mixture thus obtained was cooled to 0° C. and treated (“quenched”) with 10 mL of water.

25 mL of EtOAc was added.

The organic phase was washed 4 times with an aqueous solution composed of 1 g of LiCl in 20 mL of water and 4 times with 25 mL of water, and finally dried on cotton.

The final concentrated product obtained was purified by flash chromatography (CH₂Cl₂/EtOAc: 100/0 to 97/3) to give, at a yield of 49%, 56 mg of racemic compound 9 (GSC-028) in the form of a white solid.

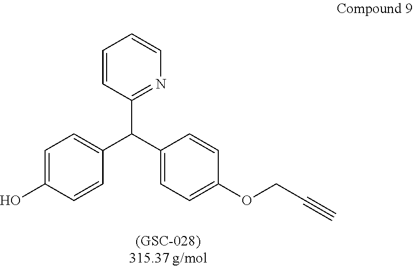 [SMILES: C#CCOc1ccc(C(c2ccc(O)cc2)c2ccccn2)cc1]

### Characteristics of Compound 9 (GSC-028)

¹H NMR (300 MHz, CDCl₃) δ 2.51 (t, 1H, J=2.0), 4.66 (d, 2H, J=2.4), 5.62 (s, 1H), 6.50 (d, 2H, J=8.1), 6.80 (d, 2H, J=8.4), 6.91 (d, 2H, J=8.4), 7.06 (d, 1H, J=8.0), 7.09 (d, 2H, J=8.7), 7.19 (t, 1H, J=6.0), 7.66 (td, 1H, J=7.6, 0.9), 8.03 (brs, 1H), 8.59 (dd, 1H, J=4.8, 0.6).

### Example 1j (Method C)

### 4-((4-(Pentan-2-yloxy)phenyl)(pyridin-2-yl)methyl)phenol (Compound 10 (GSC-029))

100 mg of 4,4′-(pyridin-2-ylmethylene)diphenol (100 mg; 360.6 μmol; 1 eq) was dissolved in 10 mL of DMF.

NaH (60% in mineral oil; 11 mg; 274.8 μmol; 1.2 eq) was added in small fractions to the reaction mixture at 0° C.

The mixture obtained was stirred until the solution reached room temperature (22° C.).

Next, a solution of 50 μl of 2-bromopentane (405.2 μmol; 1.1 eq) was added via a syringe and the contents were stirred overnight at room temperature (22° C.).

The reaction mixture thus obtained was cooled to 0° C. and treated (“quenched”) with 6 mL of water.

20 mL of CH₂Cl₂ was added.

The organic phase was washed twice with an aqueous solution composed of 1 g of LiCl in 20 mL of water and 6 times with 25 mL of water, and finally dried on cotton.

The final concentrated product obtained was purified by flash chromatography (CH₂Cl₂/EtOAc: 100/0 to 97/3) to give, at a yield of 59%, 102 mg of racemic compound 10 (GSC-029) in the form of a white solid.

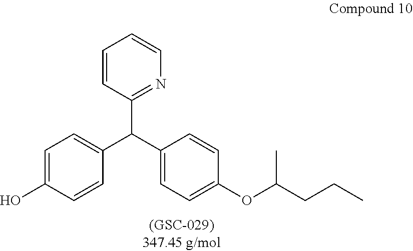 [SMILES: CCCC(C)Oc1ccc(C(c2ccc(O)cc2)c2ccccn2)cc1]

### Characteristics of Compound 10 (GSC-029)

¹H NMR (300 MHz, CDCl₃) δ 0.93 (t, 3H, J=6.9), 1.28 (d, 3H, J=6.0), 1.32-1.59 (m, 2H), 1.60-1.80 (m, 2H), 4.29-4.35 (m, 1H), 5.60 (s, 1H), 6.55 (dd, 2H, J=8.4, 0.6), 6.81 (d, 2H, J=8.7), 6.85 (d, 2H, J=8.4), 7.03 (s, 1H), 7.07 (d, 2H, J=7.8), 7.17 (t, 2H, J=6.0), 7.64 (t, 1H, J=7.5), 8.59 (dd, 1H, J=4.1, 0.8).

### Example 1k

### 4-((4-tert-Butyldimethylsilyl)(pyridin-2-yl)methyl)phenyl acetate (Compound 11 (GSC-009))

100 mg (313.1 μmol; 1 eq) of compound 3 (GSC-006) was dissolved in 3 mL of CH₂Cl₂, and treated by adding 38.8 mg of imidazole (570 μmol; 1.8 eq). 133 mg of TBDMSCl (882.4 μmol; 2.8 eq) was then added, and the resultant mixture was stirred for 4 hours at room temperature (22° C.). 20 mL of CH₂Cl₂ was added. The organic phase was washed with water (3×10 mL), dried on cotton, and concentrated under vacuum. After purification by flash chromatography (CH₂Cl₂/EtOAc: 100/0 to 80/20), racemic compound 11 (GSC-009) is obtained in the form of a white solid (117 mg, 86%).

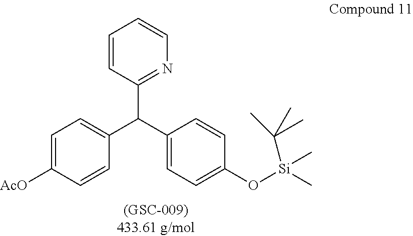 [SMILES: CC(=O)Oc1ccc(C(c2ccc(O[Si](C)(C)C(C)(C)C)cc2)c2ccccn2)cc1]

### Characteristics of Compound 11 (GSC-007)

¹H NMR (300 MHz, CDCl₃) δ 0.19 (s, 6H), 0.98 (s, 9H), 2.29 (s, 3H), 5.63 (s, 1H), 6.77 (d, 2H, J=8.7), 7.01 (d, 4H, J=8.7), 7.08 (d, 1H, J=8.1), 7.12-7.15 (m, 1H), 7.16 (d, 2H, J=8.7), 7.61 (td, 1H, J=7.6, J=1.7), 8.60 (d, 1H, J=4.8).

### Example 11

### 4-(Phenyl(pyridin-2-yl)methyl)phenol (Compound 12)

Compound 12 can be prepared according to the aforementioned Method B (Grignard):

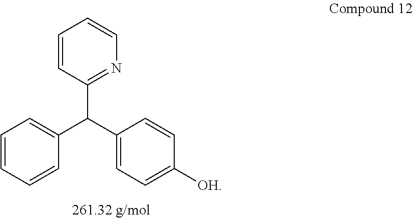 [SMILES: Oc1ccc(C(c2ccccc2)c2ccccn2)cc1]

### Example 1m

### 2-Benzhydrylpyridine (Compound 13)

Compound 13 was prepared by the aforementioned Method A (reactions catalysed by triflic acid):

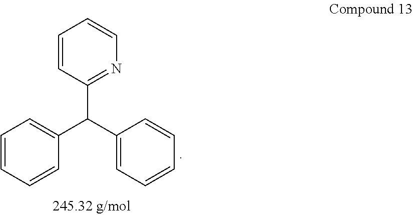 [SMILES: c1ccc(C(c2ccccc2)c2ccccn2)cc1]

### Example 1n

### 2-(bis(4-Chlorophenyl)methyl)pyridine (Compound 14)

Compound 14 was prepared by the aforementioned Method A (reactions catalysed by triflic acid):

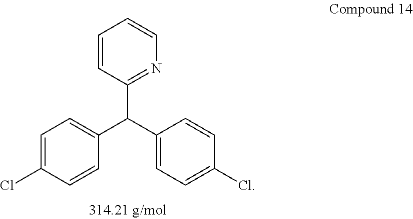 [SMILES: Clc1ccc(C(c2ccc(Cl)cc2)c2ccccn2)cc1]

### Example 1o

### 4,4′-(Pyridin-2-ylmethylene)bis(4,1-phenylene)sodium disulphate or “sodium picosulphate” (Compound 15)

This compound is a commercial product and can be purchased, for example, from the company Jai Radhe Sales, Ahmedabad, India, under the name “sodium picosulphate BP/USP” (reference: GAA/20B 8059/21B 8049).

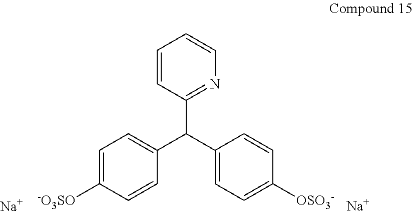

### Example 1o′

### Synthetic Methods for Additional Compounds of the Invention

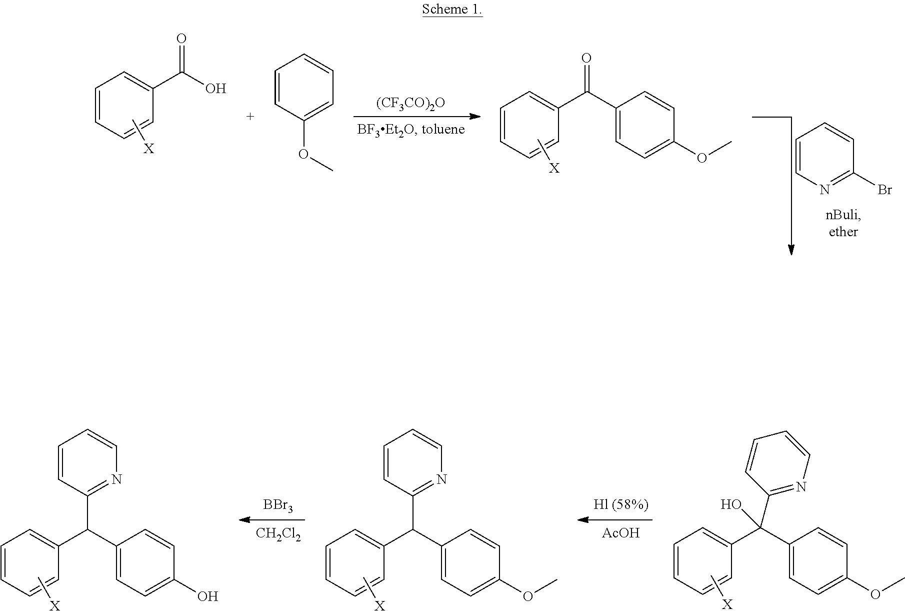

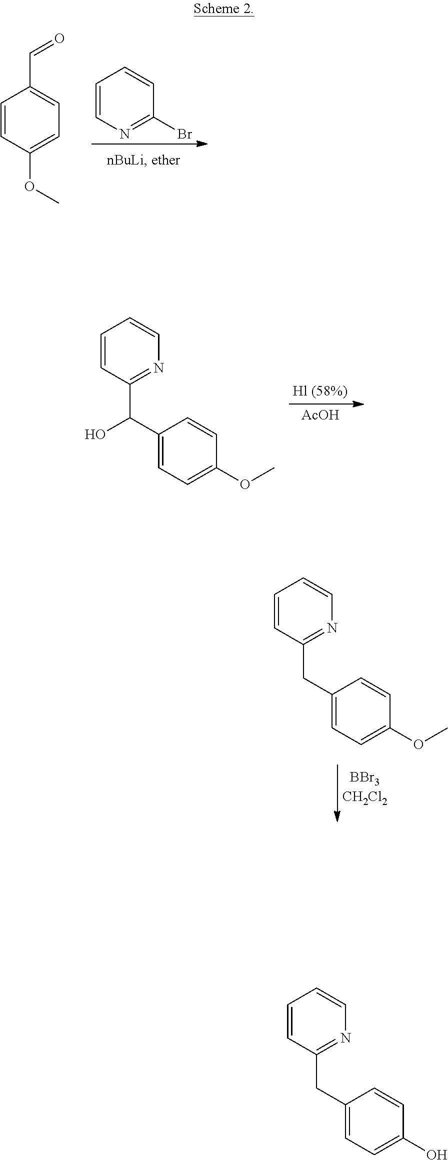

General Procedure for the benzophenone derivative synthesis (Method A)⁽⁷³⁾:

To a slurry of phenylacetic acid 1 derivative (1 equiv.) in anisol (1.3 M) was added TFAA (2 equiv.) and the solution heated to 80° C. After 60 min, BF₃.Et₂O (2%) were added, and the solution was stirred at 80° C. until complete by HPLC analysis. EtOH was added over 20 min, effecting crystallisation. The slurry was held at reflux for 1 h, cooled to 20-25 C over 60 min, then chilled to 0-5° C. The slurry was stirred for 60 min then filtered. The solids were washed with EtOH and dried in vacuo to give benzophenone derivative.

### General Procedure for the Benzophenone Alkylation (Method B)⁽⁷⁴⁾:

An ether (1 M) solution of 2-bromopyridine (1 equiv.) was added, dropwise, at −78° C. to an ether (1 M) solution of nBuLi (1.05 equiv, 1.6 M in hexanes). The solution was stirred below −40° C. (2 h). Then an ether (0.75 M) solution of benzophenone derivative (1 equiv.) was added portionwise. The solution was stirred at room temperature (16 h). A saturated aqueous NH₄Cl solution was added. The volatiles were evaporated and the residue was recrystallized in methanol or purified by column chromatography on SiO₂.

General Procedure for the De-Hydroxylation of (phenyl)(phenyl)(pyridin-2-yl)methanol (Method C)⁽⁷⁵⁾:

A solution of (phenyl)(phenyl)(pyridin-2-yl)methanol derivative (0.3 mmol), aqueous 57% HI (300 μL), and acetic acid (1.5 mL) was heated to 100° C. for 4 hours. The resulting mixture was then cooled to 0° C., basified to pH 9 with aqueous NaOH, diluted with CH₂Cl₂. The layers were separated and the organic layer was washed with a saturated aqueous Na₂S₂O₃. The organic layers were dried over MgSO₄ and the solvent was evaporated. The residue was purified by column chromatography on SiO₂.

### General Procedure for the Demethylation (Method D):

A CH₂Cl₂ solution of BBr₃ was added, dropwise, at −78° C. to a CH₂Cl₂ of methoxyphenyl derivative. The solution was stirred at room temperature (4 h). A saturated aqueous NaHCO₃ solution was added and the layers were separated. The aqueous layer was washed with CH₂Cl₂ (2 times). The organic layers were combined, dried and concentrated under vacuum. The residue was purified if necessary by column chromatography on SiO₂.

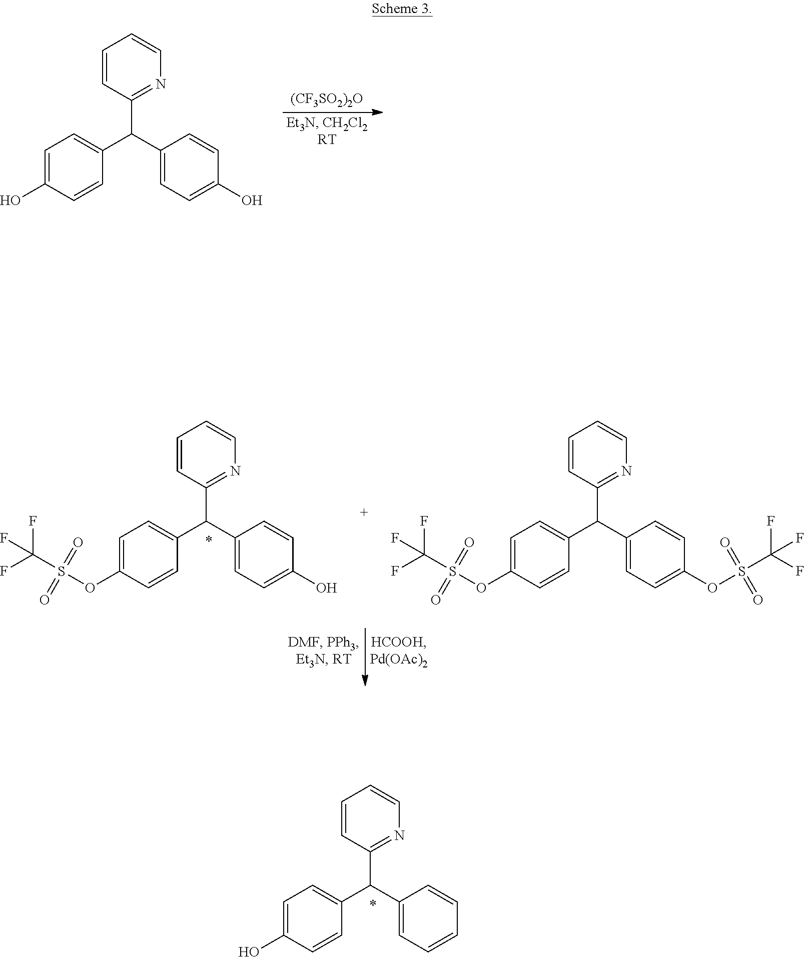

### Example 1p

### Protocol for Determining the Stability of the Test Compounds in the Culture Medium

### Preparation of a Stock Solution at 10 mM

A mass m was weighed with an analytical balance and a volume V of DMSO was added.

Incubation (n=2)

The solution of the compound at 10 mM was diluted to 1/10, still in DMSO.

The latter solution was diluted to 1/100 in medium in order to obtain a final concentration of 10 μM and 1% of DMSO.

After homogenizing (2 min), 150 μl was taken and was mixed with 150 μl of acetonitrile to precipitate the proteins present in the medium, which corresponds to a dilution to ½.

The mixture was vortexed for 1 min and then centrifuged for 10 min at 15000 g.

The supernatant was again diluted to ½ with 1 volume of milliQ water and then injected in HPLC.

The same treatment was carried out at times 2 h, 4 h, 6 h and 24 h.

A reference solution at 10 μM was prepared in water/acetonitrile 1/1 v/v.

### HPLC Analysis

The solutions were analysed in HPLC using a Gilson chain equipped with a diode array detector and of an automatic injector with an injection loop of 20 μl.

The column used was a Kinetex 2.6μ C18 100A 50×4.6 mm.

The chromatograms were recorded during injection of 20 μl of solution.

Elution was performed in gradient mode:

Flow=2 mL/min
A: water 0.1% TFA

### B: CH₃CN

### 0-0.2 min 95% A 5% B

### 2.7-3.2 min 5% A 95% B

### 3.4-6.1 min 95% A 5% B

### Example 2

### Effect of the Compounds According to the Invention on TG1 Cells

Culture of TG1 Cells

The TG1 cells were obtained and characterized as described in C. Patru, L. Romao, P. Varlet, L. Coulombel, E. Raponi, J. Cadusseau, F. Renault-Mihara, C. Thirant, N. Leonard, A. Berhneim, M. Mihalescu-Maingot, J. Haiech, I. Bieche, V. Moura-Neto, C. Daumas-Duport, M. P. Junier, H. Chneiweiss, CD133, CD15/SSEA-1, CD34 or side populations do not resume tumor-initiating properties of long-term cultured cancer stem cells from human malignant glio-neuronal tumors, BMC Cancer (2010) 10:66. and Silvestre D C, Pineda Marti J R, Hoffschir F, Studler J M, Mouthon M A, Pflumio F, Junier M P, Chneiweiss H, Boussin F D. Alternative Lengthening of Telomeres in Human Glioma Stem Cells. Stem Cells. 2011 Jan. 14. Epub ahead of print.

The TG1 proliferating cells correspond to cells for which medium is renewed regularly, twice weekly.

The TG1 cells were put into quiescence by keeping the cells in culture without changing the medium from 9 to 16 days. The state of quiescence was verified by non-incorporation of nucleotides into the DNA of the cells (see example 9).

1. Preparation of the NS34 Culture Medium of the TG1 Cells

DMEM/F12 Solution (1:1) 5×:

10 g of DMEM in powder form (Invitrogen #31600091) and 10 g of F12 in powder form (Invitrogen #21700018) were added to 400 mL of ultrapure water and the solution was filtered after dissolving the compounds in a 500 mL Stéricup®.

Glucose Solution at 15%:

15 g of glucose (Invitrogen #15023021) was dissolved completely in a small volume of ultrapure water (about 50 to 60 mL), if necessary with a magnetic stirrer. It was made up to 100 mL by adding ultrapure water. The solution was finally filtered with a 20 mL syringe equipped with a 0.22 μm filter.

NS34 (For 400 mL of Medium):

The composition of the medium comprises: 80 mL of DMEM/F12 (1:1) 5×, 16 mL of glucose solution at 15%, 4 mL of GlutaMAX-I (Invitrogen #35050038), 2 mL of buffer Hepes 1M (Invitrogen #15630056), 6 mL of sodium bicarbonate 7.5% (Invitrogen #25080060), 400 μl of penicillin/streptomycin (Invitrogen #15140148), 4 mL of N2 (Invitrogen #17502048), 4 mL of G5 (Invitrogen #17503012), 4 mL of B27 (Invitrogen #17504044). Ultrapure water was added to 400 mL and the solution was filtered in a 500 mL Stéricup®.

2. Procedure for Dividing the Cells:

This procedure was performed twice weekly.

The whole culture was collected using a 10-mL pipette in a 50-mL Falcon tube, then centrifuged for 10 minutes at 800 rpm. The supernatant was recovered in another 50-mL Falcon tube, this supernatant constitutes the conditioned medium of the TG1 cells.

Mechanical dissociation was carried out. 1 mL of the conditioned medium was put back in the tube containing the cell pellet. The cell pellet was dissociated mechanically by pipetting up and down 100 times with a P1000 micropipette preset to 400 μl.

For counting, the cells were diluted about 4 times in the conditioned medium. 20 μl of medium with the cells was taken before sedimentation of the cells. 4 μl of Trypan Blue was added and 20 μl of this mixture was put in a counting cell Quick-Read Precision Cell Slide (Globe Scientific Inc.). The number of cells contained in the 18 circles was counted, and the number of cells per mL was determined from the following formula: Number of cells/mL=(N/18)×10³/0.011 where N corresponds to the number of cells counted.

After the calculations has been carried out, the concentration of the cells was adjusted to 2.5×10⁶ cells/mL by adding conditioned medium.

90% of freshly prepared NS34 medium was put in a flask and 10% of conditioned medium containing the cells at 2.5×10⁶ cells/mL was added. Finally the flask was incubated at 37° C. with 5% CO₂ under humid atmosphere (at 95% provided by a reservoir filled with distilled water).

3. Preparation of the Cells for the Test of Response to the Compounds According to the Invention

The proliferating cells were dissociated and put back in fresh medium the previous day, following the above protocol.

The cells (proliferating and quiescent) were centrifuged separately for 10 minutes at 800 rpm. The supernatant of the proliferating and quiescent cells was removed completely.

The proliferating cells were put back in 1 mL of fresh NS34 medium, dissociated mechanically by pipetting up and down 100 times with a P1000 micropipette preset to 400 μl and counted with Trypan Blue, after dilution in fresh NS34 medium, to obtain a final suspension of 6×10⁵ cells/mL.

The quiescent cells were put back in 1 mL of conditioned medium, dissociated mechanically by pipetting up and down 100 times with a P1000 micropipette preset to 400 μl and counted with Trypan Blue, after dilution in conditioned medium, to obtain a final suspension of 8.10⁵ cells/mL.

7 mL of cellular suspension was prepared for each test compound.

4. Protocol for Testing the Dose/Response of the Compounds

50 μl of solution containing cells was deposited per well in a 96-well plate (Greiner #655090).

The test compounds in the form of powder were weighed in 1.5-mL microtubes on a precision balance at the rate of 1 to 3 mg. The exact value was noted and the compound was dissolved in 100% of DMSO (Sigma #154938) to a final concentration of 10 mM.

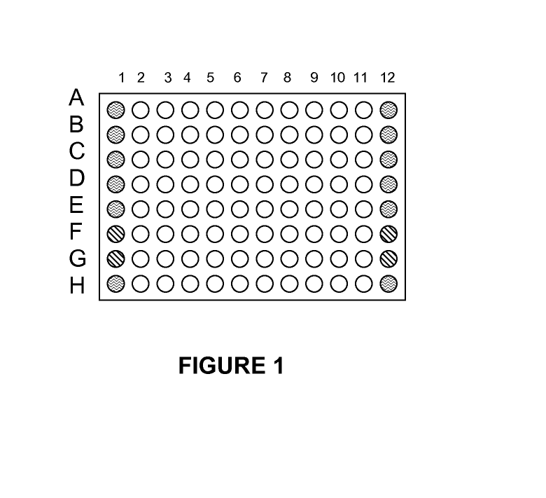

The test compounds, in solution of 100% of DMSO were diluted in culture medium: in the absence of DMSO (medium 0% DMSO) or in the presence of 2% of DMSO (medium 2% DMSO) at concentrations of 200 μM, 100 μM, 80 μM, 60 μM, 40 μM, 20 μM, 10 μM and 2 μM. For all these dilutions the final concentration in DMSO was 2% (see Table 1 below; volumes shown make it possible to construct dose/response curves, in triplicate, for 1 compound on a 96-well plate—FIG. 1)

TABLE 1

| | | | Concentration after |
|---|---|---|---|
| Concentration to be | Volume of | | putting in contact with |
| prepared | compound | Volume of medium | the cells |
| 200 μM; 2% DMSO | 10.8 μl of 10 mM | 529.2 μl of medium 0% DMSO | 100 μM; 1% DMSO |
| 100 μM; 2% DMSO | 110 μl of 200 μM | 110 μl of medium 2% DMSO | 50 μM; 1% DMSO |
| 80 μM; 2% DMSO | 80 μl of 200 μM | 120 μl of medium 2% DMSO | 40 μM; 1% DMSO |
| 60 μM; 2% DMSO | 60 μl of 200 μM | 140 μl of medium 2% DMSO | 30 μM; 1% DMSO |
| 40 μM; 2% DMSO | 40 μl of 200 μM | 160 μl of medium 2% DMSO | 20 μM; 1% DMSO |
| 20 μM; 2% DMSO | 22 μl of 200 μM | 198 μl of medium 2% DMSO | 10 μM; 1% DMSO |
| 10 μM; 2% DMSO | 20 μl of 100 μM | 180 μl of medium 2% DMSO | 5 μM; 1% DMSO |
| 2 μM; 2% DMSO | 20 μl of 20 μM | 180 μl of medium 2% DMSO | 1 μM; 1% DMSO |

The positive control and negative control were prepared respectively with a solution of terfenadine at 100 μM; 2% DMSO in the culture medium and with a solution 2% DMSO in the culture medium.

50 μL of solution of compound was added at the concentrations specified above to 50 μL of cells in the plate (FIG. 1) (Final concentration of compounds in contact with the cells: 100 μM, 50 μM, 40 μM, 30 μM, 20 μM, 10 μM, 5 μM, 1 μM (in 1% of final DMSO). Final concentration of positive control: 50 μM in 1% of final DMSO. Final concentration of negative control: 1% final DMSO).

The cells were incubated for 23 to 24 hours at 37° C. in an incubator in the presence of 5% CO₂ (under humid atmosphere at 95% provided by a reservoir filled with distilled water).

The test of cell viability was carried out using the Cell Titer Glo kit (Promega #G7571). After thawing of the Cell Titer Glo reagent (1 Buffer vial+1 Substrate vial per plate) protected from the light, the contents of the Buffer vial were transferred to the Substrate vial. 100 μl of Cell Titer Glo reagent was deposited on the cells treated with the compounds.

The plates were covered with aluminium and stirred for 2 minutes. After 10 minutes of stabilization of the luminescence, the luminescent signal was measured with a Victor 3 reader.

The results, which were given for each well in strokes per second (sps), were converted to percentage cell survival relative to the control 1% of DMSO, which was fixed at 100%.

The EC₅₀ was determined as the concentration that gives 50% of the response.

5. Results of the Dose/Response Test of the Compounds

See discussion in Examples 10 and 11.

### Example 3

### Effect of the Compounds According to the Invention on OB1 Cells

1. Culture of the OB1 cells
See Example 2.1 to 2.3

2. Protocol of dose/response test of the compounds
See Example 2.4

3. Results of the dose/response test of the compounds

See discussion in Examples 10 and 11.

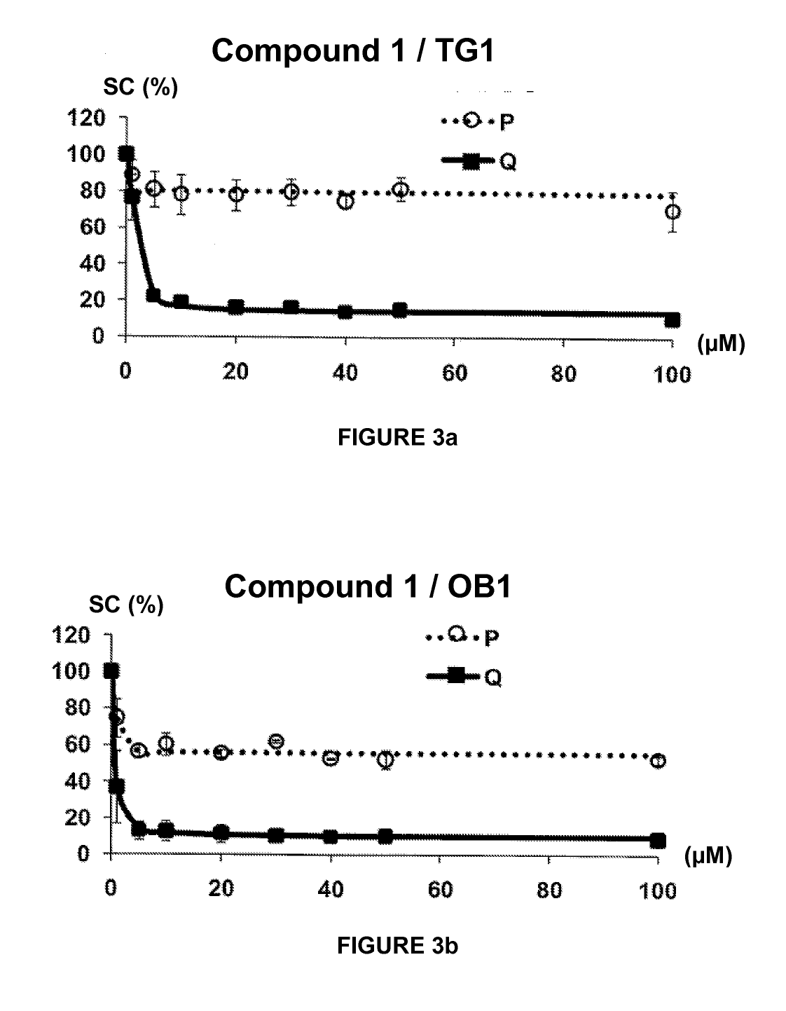

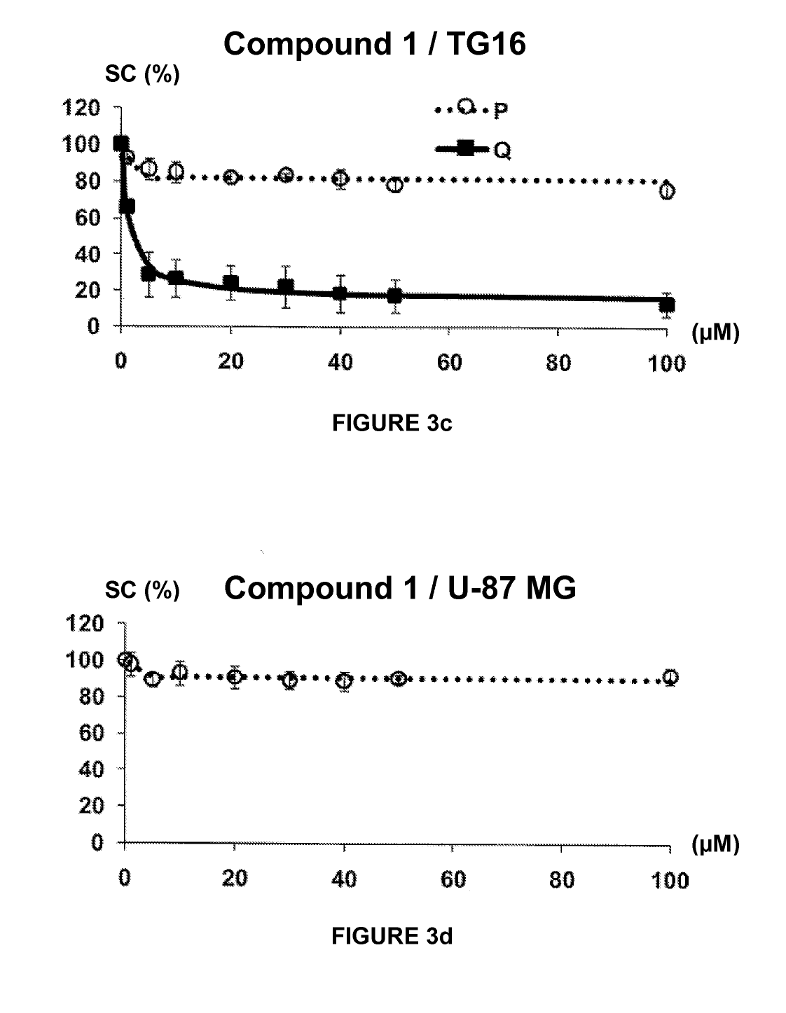

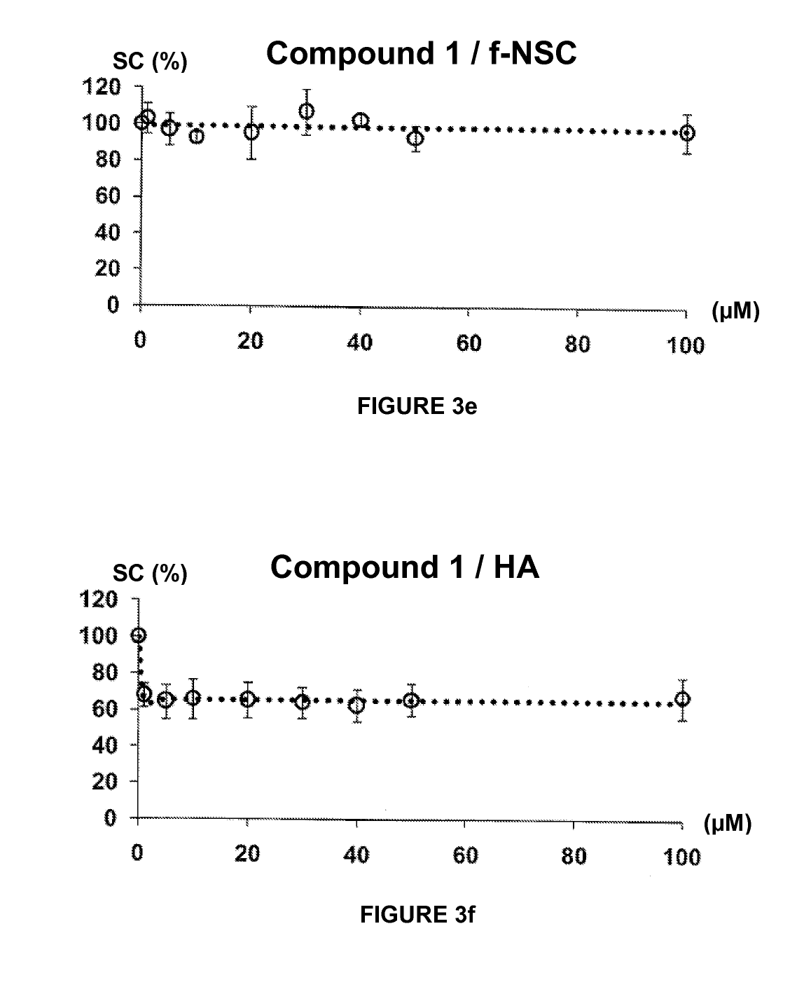

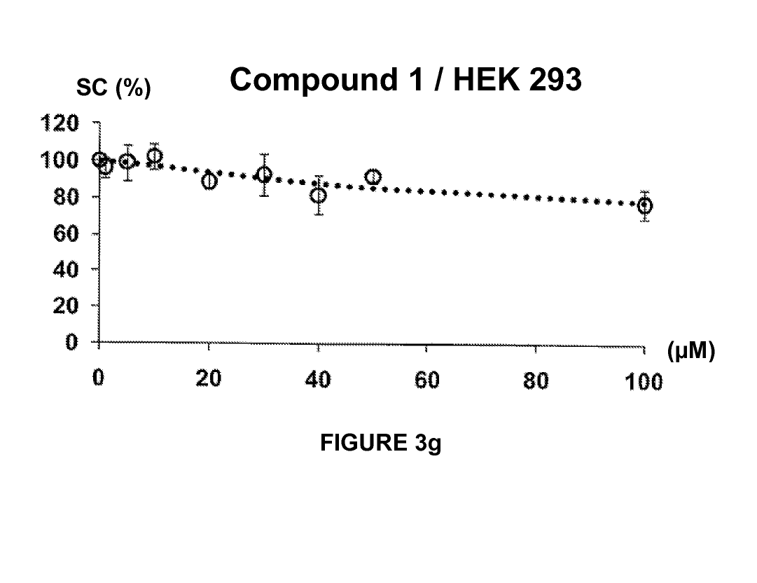

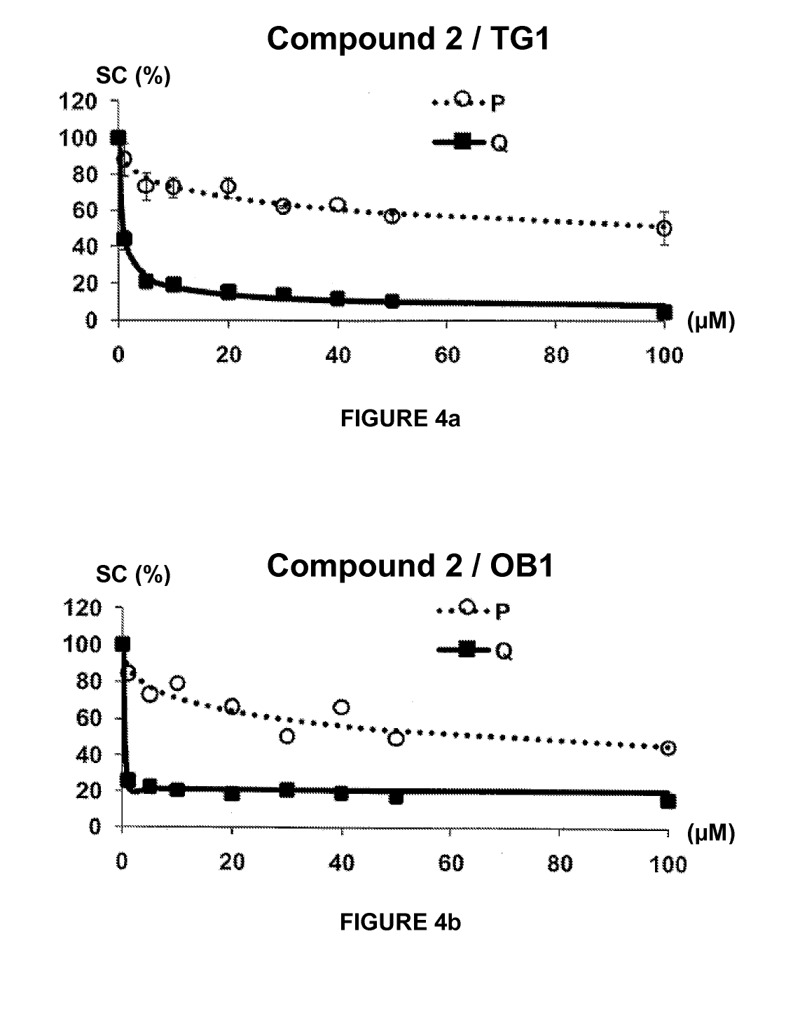

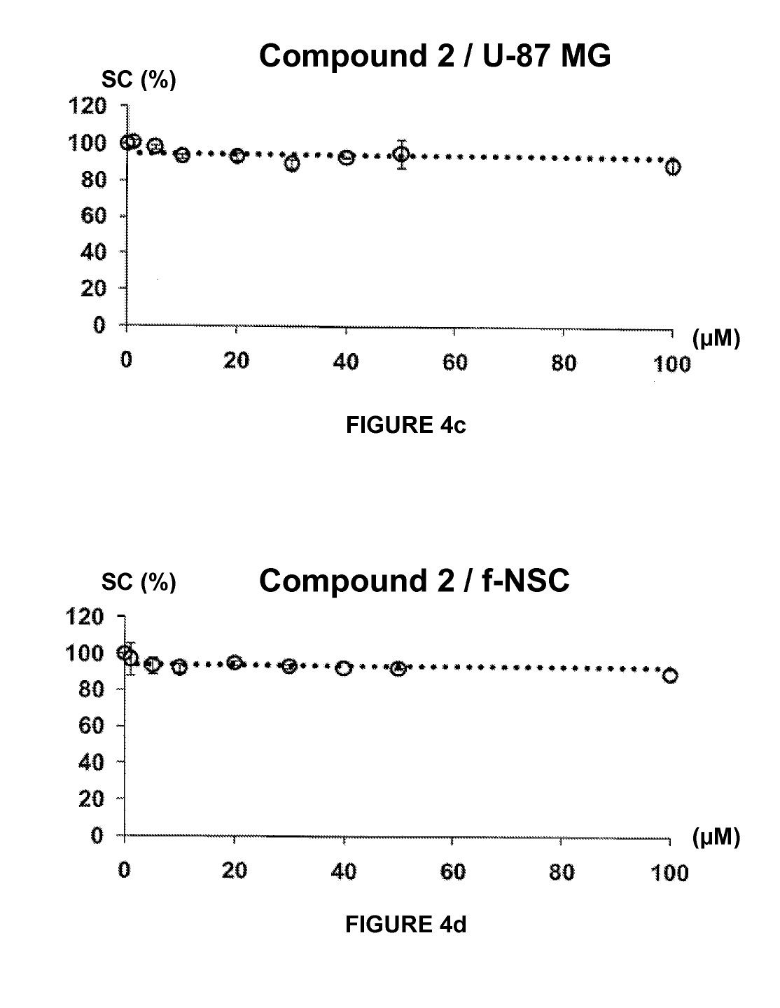

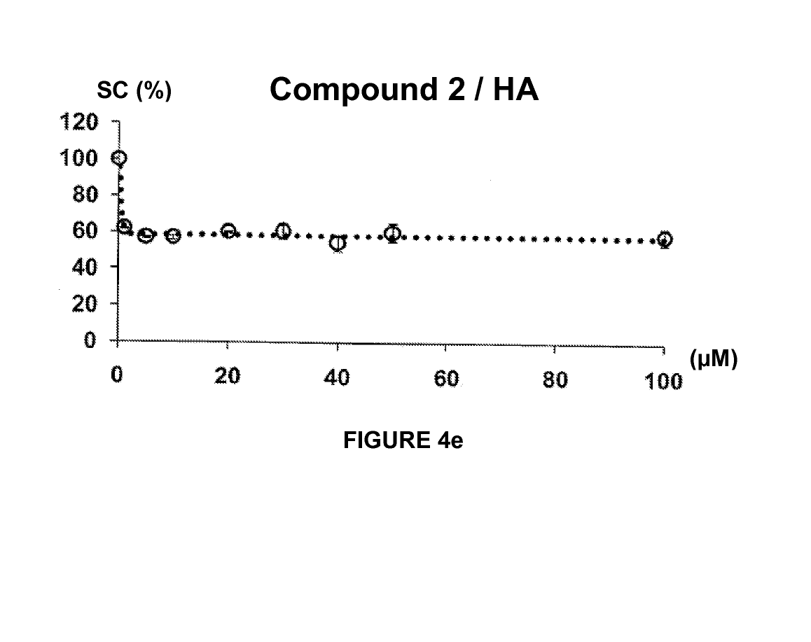

(FIGS. 3b and 4b)

### Example 4

### Effect of the Compounds According to the Invention on TG16 Cells

1. Culture of TG16 cells
See Example 2.1 to 2.3

2. Protocol of dose/response test of the compounds
See Example 2.4

3. Results of the dose/response test of the compounds

See discussion in Examples 10 and 11.
(FIG. 3c)

### Example 5

### Effect of the Compounds According to the Invention on Human Fetal Neural Cells: f-NSC Cells

Culture of the f-NSC Cells

1. Preparation of the Complete Culture Medium of the f-NSC Cells (for 10 mL)

The culture medium is composed of 9 mL of NeuroCult® NSC Basal Medium (Mouse) (Stem Cell Technologies #05700), 1 mL of NeuroCult® NSC Proliferation Supplement (Stem Cell Technologies #05701), 1 μl of Recombinant Human FGF-Basic (Peprotech France #AF-100-18B) at a concentration of 100 μg/mL and 20 μl of Recombinant Human EGF (Peprotech France #AF-100-15) at a concentration of 10 μg/mL.

2. Procedure for Dividing the Cells

This procedure was carried out every 11 days when the cells are in the form of neurospheres of medium size (about 100 cells per sphere):

The whole culture was collected using a 10-mL pipette in a 50-mL Falcon tube, then centrifuged for 10 minutes at 800 rpm. The supernatant was recovered in another 50-mL Falcon tube, this supernatant constitutes the conditioned medium of the NSCs.

Mechanical dissociation, or with the kit: NeuroCult® Chemical Dissociation (Stem cell Technologies #05707) was performed on the cell pellet:

For mechanical dissociation, 1 mL of the conditioned medium was put back in the tube containing the cell pellet. The cell pellet was dissociated mechanically by pipetting up and down 50 to 100 times with a P1000 micropipette preset to 400 μl.

For chemical dissociation with the NeuroCult® Chemical Dissociation kit, 1 mL of solution A (Stem cell Technologies #05707A) was added to the pellet. The cells were resuspended by aspirating and discharging 8 times, then 250 μl of solution B (Stem cell Technologies #05707B) was added. An 8-minute count was performed, at minutes 3 and 7 the cellular suspension was mixed by aspirating and discharging 8 times. At the end of the 8 minutes, 80 μl of solution C (Stem cell Technologies #05707C) was added, and the suspension was once again mixed 8 times. Finally 700 μl of complete culture medium was added for a final volume of 2 mL.

For counting, the cells were diluted by a factor of 3 in conditioned medium. 20 μl of medium with the cells was taken before sedimentation of the cells. 4 μl of Trypan Blue was added and 20 μl of this mixture was put in a counting cell Quick-Read Precision Cell Slide (Globe Scientific Inc.). The number of cells contained in the 18 circles was counted, and number of cells per mL was determined from the following formula: Number of cells/mL=(N/18)×10³/0.011, where N corresponds to the number of cells counted.

Once the calculations had been made, the cells were put at a concentration of 2.5 to 3×10⁶ cells/mL in the Falcon tube by adding conditioned medium.

90% of freshly prepared medium was put in a T75 NUNC flask, and 10% of conditioned medium containing the cells at 2.5 to 3×10⁶ cells/mL was added. Finally the flask was incubated at 37° C. with 5% CO₂ under humid atmosphere at 95% provided by a reservoir filled with distilled water.

3. Preparation of the Cells for Testing the Response to the Compounds

The cells were passed 1 week before the experiment, that very same day, the cells were centrifuged for 10 minutes at 800 rpm. The supernatant was removed and the cells were taken up in complete culture medium.

The cells were dissociated by the chemical method (see above) and sieved in order to obtain a homogeneous cellular suspension.

The cells were counted and diluted to obtain a final suspension of 20×10⁶ cells/mL.

7 mL of cellular suspension at 20×10⁶ cells/mL was prepared for each test compound.

4. Protocol of Dose/Response Test of the Compound

See Example 2.4

5. Results of the Dose/Response Test of the Compound

The effect of bisacodyl (compound 1) was tested on neural stem cells isolated from human fetal brain (f-NSC cells) (FIG. 3e).

It was found that bisacodyl (compound 1) is practically without effect on the fetal neural stem cells with over 90% cell survival, for concentrations of compound up to 100 μM.

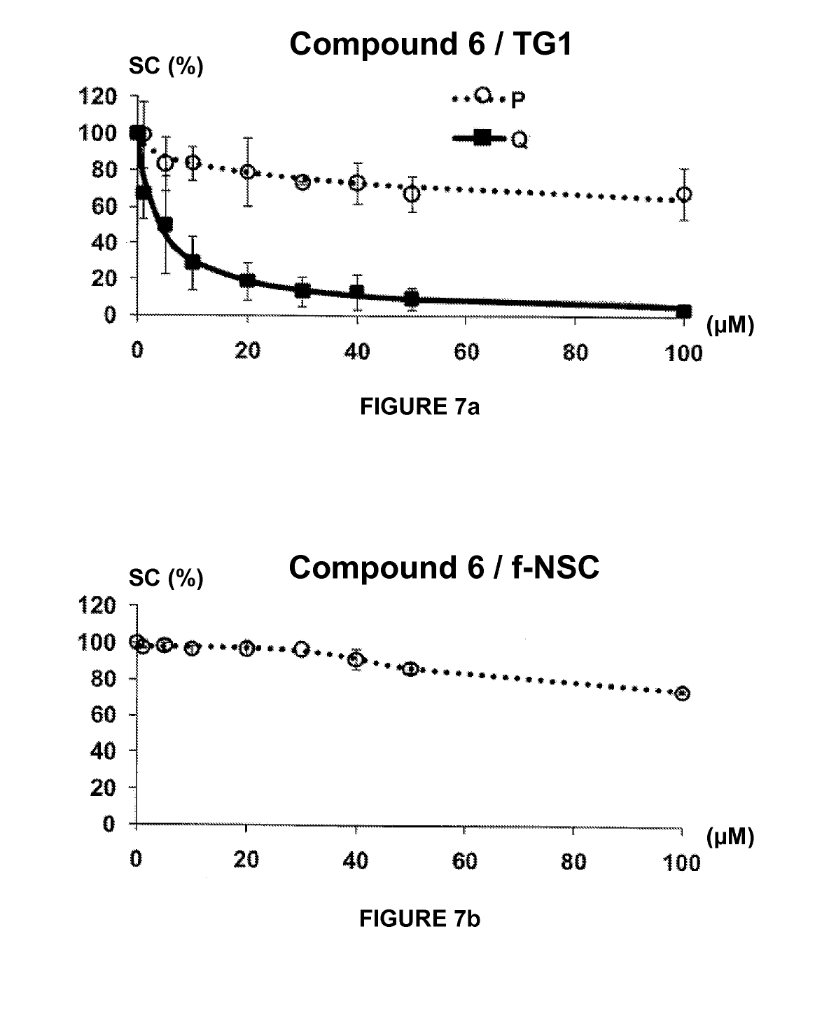

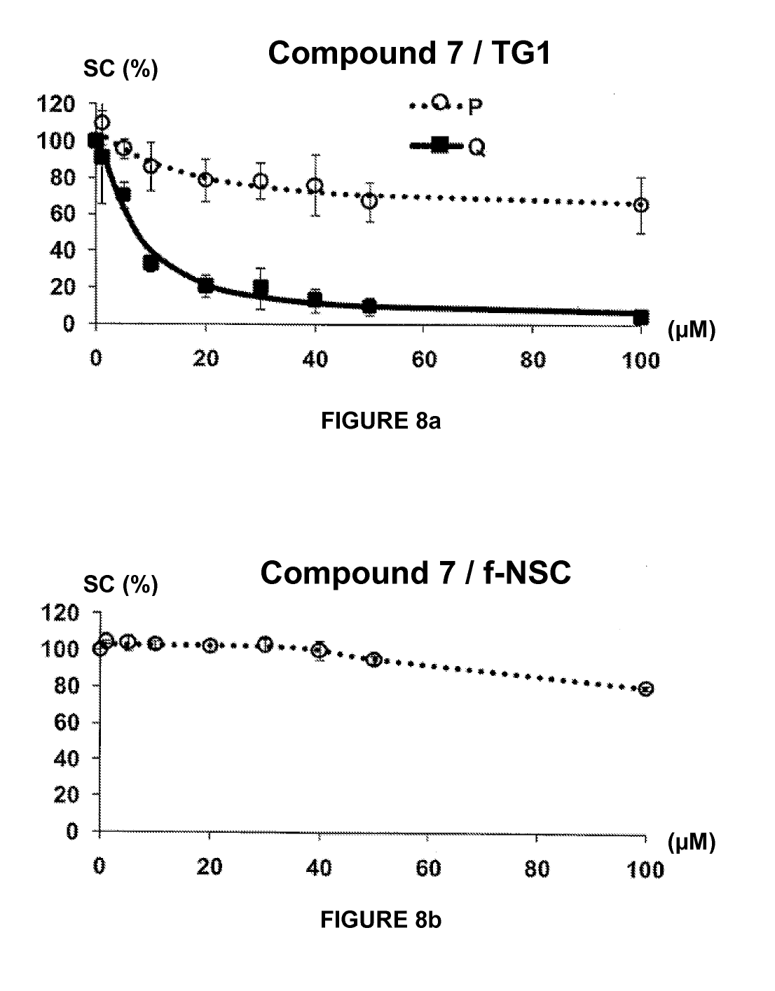

See also discussion in Examples 10 and 11, and FIGS. 4d, 5b, 7b, 8b, 9b and 10b.

### Example 6

### Effect of the Compounds According to the Invention on Human Astrocyte (HA) Cells

Culture of the Human Astrocyte (HA) Cells

1. Preparation of the Human Astrocyte Culture Flask

10 mL of sterile-filtered milliQ water was put in a Falcon T75 culture flask, to which 15 μl of stock solution of poly-L-lysine at 10 mg/mL was added (Poly-L-Lysine reference PLL Innoprot).

The flask was left in the incubator overnight at 37° C.

2. Thawing of the Human Astrocytes

Culture medium was prepared with the Astrocyte Medium culture medium kit from ScienCell (#1801) according to the manufacturer's instructions. The culture medium comprises 500 mL of base medium, 10 mL of fetal calf serum (ScienCell, #0010), 5 mL of growth supplement for astrocytes (ScienCell, #1852) and 5 mL of penicillin/streptomycin solution (ScienCell, #0503).

The poly-L-lysine was removed from the previously prepared flask, and the flask was rinsed twice with sterile-filtered milliQ water. 20 mL of medium was added.

The vial of human astrocyte cells was thawed in a water bath at 37° C. The cell clusters and pellet were resuspended gently and the contents of the vial were transferred to the previously prepared flask. The cells were distributed throughout the flask.

The cells were incubated in an incubator at 37° C.; 5% CO₂; humid atmosphere at 95% provided by a reservoir filled with distilled water.

On the next day, the medium was changed to remove the DMSO and the dead cells from the flask.

The medium was changed every other day until the culture was at 50% confluence, and every day until it was at 80% confluence.

The first passage was effected at 90% confluence.

3. Passaging the Human Astrocyte Cells

The solutions of trypsin-EDTA (Invitrogen, #25200-056), of the culture medium and of phosphate-buffered saline (PBS) with the following composition: 8 g/l of NaCl, 0.2 g/l of KCl, 0.2 g/l of KH₂PO₄ and 1.15 g/l of Na₂HPO₄, 7 H₂O, were preheated to room temperature (25° C.).

The medium was removed from the flasks and the flasks were rinsed with PBS.

The cells were incubated with 1 to 2 mL of trypsin until 80% of the cells are round.

10 mL of culture medium was added to inhibit the trypsin.

The cells were transferred to a 50-mL Falcon tube, and rinsed once with 10 mL of medium.

The cells collected were centrifuged at 1000 rpm for 5 minutes, the medium was discarded and the cell pellet was resuspended in 10 mL of culture medium.

The flasks were seeded with 4×10⁶ cells and incubated at 37° C., 5% CO₂.

The medium was changed every other day until the culture was at 50% confluence, and every day until it was at 80% confluence.

4. Cell Counting Procedure

For counting, 20 μl of medium with the cells was taken before sedimentation of the cells. 4 μl of Trypan Blue was added and 20 μl of this mixture was deposited in a Quick-Read Precision Cell Slide counting cell (Globe Scientific Inc.). The number of cells contained in 18 circles was counted, and the number of cells per mL was determined from the following formula: Number of cells/mL=(N/18)×10³/0.011, where N corresponds to the number of cells counted.

5. Preparation of the Cells for the Test of Response to the Chemical Compounds

The cells were passed 2 or 3 days before the experiment. That very day, the cells are detached with trypsin according to the above protocol, then centrifuged for 10 minutes at 800 rpm.

The supernatant was removed and the cells were resuspended in culture medium, then counted and finally diluted to give a final suspension of 10⁶ cells/mL.

7 mL of cellular suspension at 10⁶ cells/mL was prepared for each test compound.

6. Protocol of Test of Dose/Response to the Compounds

See Example 2.4

7. Results of the Test of Dose/Response to the Compounds

It was found that bisacodyl (compound 1) causes a moderate decrease in survival of human astrocytes (HA) in primary culture with 60% survival for concentrations above 10 μM (FIG. 3f).

See also discussion in Examples 10 and 11 and FIG. 4e.

### Example 7

### Effect of the Compounds According to the Invention on Commercial U-87 MG Cells from Glioblastomas

Culture of the U-87 MG Cells

1. Preparation of the Culture Medium of the U-87 MG Cells

The culture medium was prepared from a 500-mL bottle of Eagle's Minimum Essential Medium (EMEM) (ATCC, #30-2003), to which 50 mL of fetal calf serum (FBS) (Invitrogen, #10270-106) and 5 mL of penicillin/streptomycin (Invitrogen, #15070-063 or Merck-Polylabo, #60703) were added.

The complete medium was stored at 4° C.

2. Procedure for Dividing the Cells

This procedure was carried out once or twice weekly for a T75 flask (Falcon).

The culture medium was removed using a 10-mL pipette and the cell lawn was rinsed with 5 mL of phosphate-buffered saline (PBS) with the following composition: 8 g/l of NaCl, 0.2 g/l of KCl, 0.2 g/l of KH₂PO₄ and 1.15 g/l of Na₂HPO₄, 7H₂O.

10 mL of PBS was added gently, the flask was stirred briefly and gently and then the PBS was removed using a 5-mL pipette.

1 mL of Trypsin-EDTA was added to the cells and incubated for a few minutes at room temperature (25° C.), long enough for the cells to detach from the flask. To prevent the cells aggregating together, the flask must not be shaken.

9 mL of culture medium was added to the flask and solution was homogenized by gently pipetting up and down in a 10-mL pipette.

3-mL aliquots of this suspension were added to new flasks containing 7 mL of culture medium so as to have 10 mL of final volume per flask.

The cells were incubated at 37° C. with 5% CO₂ under humid atmosphere at 95% provided by a reservoir filled with distilled water.

3. Preparation of the Cells for the Test of Response to the Compounds

The cells were passed 2 or 3 days before the experiment. On that very day, the cells are detached with trypsin according to the above protocol, then centrifuged for 10 minutes at 800 rpm.

The supernatant was removed and the cells were resuspended in culture medium, then counted and finally diluted to give a final suspension of 10⁶ cells/mL.

7 mL of cellular suspension at 10⁶ cells/mL was prepared for each test compound.

4. Protocol for the Test of Dose/Response to the Compounds

See Example 2.4

5. Results of the Test of Dose/Response to the Compounds

It was found that the U-87 MG cells of the human glioblastoma cell line, corresponding to proliferating cancer cells, are only very slightly affected by bisacodyl (compound 1) with 90% survival for concentrations between 10 and 100 μM (FIG. 3d).

See also discussion in Examples 10 and 11, and FIG. 4c.

### Example 8

### Effect of the Compounds According to the Invention on HEK 293 Cells

Culture of the HEK 293 Cells

1. Preparation of the Culture Medium of the HEK 293 Cells

The following were added to a 500-mL bottle of Minimum Essential Medium (MEM) (Invitrogen #32561-037): 50 mL of solution of fetal calf serum (FBS) (Invitrogen #10270-106) and 5 mL of penicillin/streptomycin solution (Invitrogen #15070-063).

2. Procedure for Dividing the Cells:

This procedure was carried out once or twice weekly for a T75 flask (Falcon), the time for generating the HEK 293 cells is from 3 to 4 days.

The culture medium was removed using a 10-mL pipette and the cell lawn was rinsed with 5 mL of phosphate-buffered saline (PBS) with the following composition: 8 g/l of NaCl, 0.2 g/l of KCl, 0.2 g/l of KH₂PO₄ and 1.15 g/l of Na₂HPO₄, 7 H₂O.

10 mL of PBS was added gently, the flask was stirred briefly and gently and then the PBS was removed using a 5-mL pipette.

1 mL of Trypsin-EDTA was added to the cells and incubated for a few minutes at ambient temperature of 25° C., long enough for the cells to detach from the flask. To prevent the cells aggregating together, the flask must not be shaken.

9 mL of culture medium was added to the flask and the solution was homogenized by gently pipetting up and down in a 10-mL pipette.

3-mL aliquots of this suspension were added to new flasks containing 7 mL of culture medium so as to have 10 mL of final volume per flask.

The cells were incubated at 37° C. with 5% CO₂ under humid atmosphere at 95% provided by a reservoir filled with distilled water.

3. Preparation of the Cells for the Test of Response to the Compounds

The cells were passed 2 or 3 days before the experiment. On that very day, the cells are detached with trypsin according to the above protocol, then centrifuged for 10 minutes at 800 rpm.

The supernatant was removed and the cells were resuspended in culture medium, then counted and finally diluted to give a final suspension of 10⁶ cells/mL.

7 mL of cellular suspension at 10⁶ cells/mL was prepared for each test compound.

4. Protocol for Testing Dose/Response to the Compounds

See Example 2.4

5. Results of the Test of Dose/Response to the Compounds

The effect of bisacodyl (compound 1) was tested on lines of human embryo kidney cells in culture (HEK293 cells) (FIG. 3g).

At a concentration of 10 μM, bisacodyl (compound 1) has no effect on the HEK293 cells (100% survival) and its effect is not very pronounced at higher concentrations (80% survival at 100 μM).

See also discussion in Examples 10 and 11.

### Example 9

### Test of Cellular Proliferation by Incorporation of 5-Ethynyl-2′-deoxyuridine (EdU)

Preparation of the Cells for Incubation with EdU

For each condition (quiescence and proliferation), 2 samples between 3 and 5 mL of cellular suspension were taken and put in two T25 flasks.

10 μM of EdU was added to one of the two flasks; the other flask being the control flask without EdU.

Therefore the following were obtained:
one T25 Proliferating Cells+10 μM EdU
one T25 Proliferating Cells
one T25 Quiescent Cells+10 μM EdU
one T25 Quiescent Cells

The flasks were incubated for 24 hours at 37° C. with 5% CO₂, under humid atmosphere at 95% provided by a reservoir filled with distilled water.

Labelling of the Cells:

The cells that had been incubated with EdU were recovered in two 14-mL tubes.

The cells were centrifuged at 1500 rpm for 5 min and the supernatant was removed.

The cells were washed once with 5 mL of PBS solution containing 1% of BSA, first dissociating them in 1 mL.

The cells were centrifuged at 1500 rpm for 5 min and the supernatant was removed.

The cells were fixed with 50 μL of fixative (component D of the kit Click-iT EdU Flow Cytometry Alexa Fluor® 488 Azide Invitrogen), the pellet was dissociated and the homogeneity of the cellular suspension was verified.

The cellular suspension was incubated for 15 min at ambient temperature of 25° C. in the dark.

The cells were washed once with 3 mL of PBS solution containing 1% of BSA.

The cells were centrifuged at 1500 rpm for 5 min and the supernatant was removed.

100 μL of permeabilization solution (component E of the kit Click-iT EdU Flow Cytometry Alexa Fluor® 488 Azide Invitrogen) was added and the suspension was mixed so as to obtain a homogeneous suspension.

500 μL of reaction cocktail prepared extemporaneously was added with, for 1 reaction:

TABLE 2

| reagent | Volumes (μL) |
|---|---|
| 1x Click-iT ™ reaction buffer | 438 |
| (component G) | |
| CuSO₄ (component H) | 10 |
| Azide fluorescent dye (component B) | 2.5 |
| Additive of reaction buffer (component | 50 |
| I) | |
| Total volume | 500 |

The suspension was incubated for 30 min at ambient temperature of 25° C. in the dark.

The cells were washed once with 3 mL of washing and permeabilization solution (component E of the kit Click-iT EdU Flow Cytometry Alexa Fluor® 488 Azide Invitrogen).

The cells were centrifuged at 1500 rpm for 5 min and the supernatant was removed.

500 μL of washing and permeabilization solution (component E of the kit Click-iT EdU Flow Cytometry Alexa Fluor® 488 Azide Invitrogen) was added.

The cells were put in 96-well plates suitable for the Guava capillary cytometer: 200 μL per well in duplicate.

2 μL of 7-AAD (present in the kit Click-iT EdU Flow Cytometry Alexa Fluor® 488 Azide Invitrogen) was added to each well, changing the cone each time.

Viability of the Cells:

In parallel with the 30-min incubation of the cells that had incorporated EdU, the viability of the cells was tested by incorporation of 7-AAD.

The proliferating and quiescent cells not labelled with EdU were recovered in two 14-mL tubes.

The cells were centrifuged at 1500 rpm for 5 min and the supernatant was removed.

The cells were washed once with 5 mL of PBS solution containing 1% of BSA, first dissociating them in 1 mL.

The cells were centrifuged at 1500 rpm for 5 min and the supernatant was removed.

The cells were taken up in 500 μL of PBS solution containing 1% of BSA.

The cells were put in 96-well plates suitable for the Guava capillary cytometer: 200 μL per well in duplicate.

2 μL of 7-AAD (present in the kit) was added to each well, changing the cone each time.

Reading of the Cells in the Guava Capillary Cytometer:

The CytoSoft software and the Express Pro program were used
Excitation: 448 nm
Emission at 530/30 nm for Alexa Fluor 488
Emission at 660/20 nm for CellCycle 488-red (7-AAD)

### Example 10

### Effect of Bisacodyl and of 4,4′-Dihydroxy-Diphenyl-(2-Pyridyl)-Methane (DDPM) (or Compound 2 (GSC-002)) on the Various Cellular Types Mentioned Above Apart from TG16

### Bisacodyl: Compound 1

Bisacodyl, or 4,4′-diacetoxydiphenyl)(2-pyridyl)methane or acetic acid 4-[(4-acetoxy-phenyl)-pyridin-2-yl-methyl]-phenyl ester (DAMP), corresponds to the following formula:

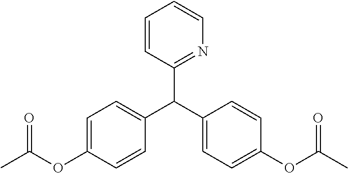 [SMILES: CC(=O)Oc1ccc(C(c2ccc(OC(C)=O)cc2)c2ccccn2)cc1]

This compound is used in therapeutics for its laxative properties by the oral route or by the rectal route. The molecule has no known toxicity.

Bisacodyl (compound 1) has two esterified phenol groups. Data from the literature indicate that bisacodyl (compound 1) would be a prodrug, the active compound being 4,4′-dihydroxy-diphenyl-(2-pyridyl)-methane or DDPM.

The stability of bisacodyl (compound 1) in the culture medium has been tested (example 1bis).

It was shown that bisacodyl (compound 1) (Compound 1 (GSC-001)) was decomposed to a metabolite with a half-life of 2 hours in the medium of the proliferating cells and of 4 hours in the medium of the quiescent cells.

FIG. 3a shows the effect of bisacodyl on cancer stem cells isolated from a patient (TG1 cells).

The cells are cultivated in DMEM:F12 (1:1) medium in the presence of supplements N2, G5 and B27 from Invitrogen. The proliferating cells correspond to cells for which the medium is renewed regularly. The TG1 cells are put into quiescence by keeping the cells in culture without changing the medium for 16 days. The state of quiescence is verified by non-incorporation of nucleotides into the DNA of the cells.

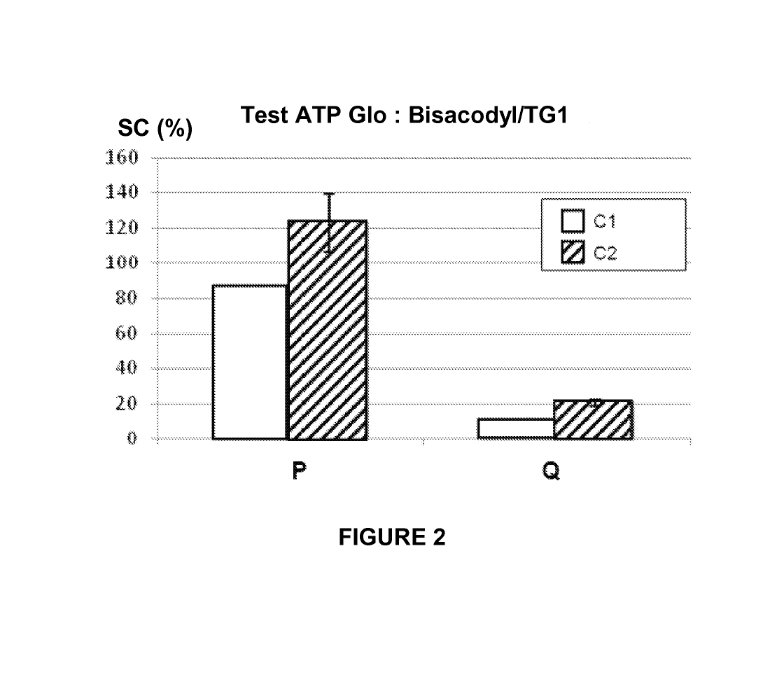

Bisacodyl (compound 1) is only active on TG1 cells in quiescence (and not on TG1 cells proliferating continuously), i.e. cells that do not enter the cell cycle in phase S, but remain blocked in a phase called G0/G1 but are still alive (FIGS. 2 and 3a). The value of the concentration of bisacodyl (compound 1) leading to 50% of effect (EC₅₀) is 1 μM.

The effects of bisacodyl on the TG1 stem cells were also found on cancer stem cells isolated from glioblastomas of two other patients (TG16 and OB1 cells).

The effect of bisacodyl was also tested on neural stem cells isolated from human fetal brain (f-NSC cells), on human primary astrocytes (HA cells) and on lines of human embryonic kidney cells in culture (HEK293 cells). Bisacodyl does not display any toxicity for these three types of cells.

The current standard treatment for glioblastoma is TEMODAL (temozolomide or TMZ), which is an alkylating agent of DNA. Its use permits an increase in patient survival by about 2 months. TMZ has an antiproliferative action but is unable to remove all the cancer cells. Cancer stem cells are resistant to the treatment.

Bisacodyl is the first molecule that acts on quiescent cancer stem cells.

Thus, it has been demonstrated in the present invention that bisacodyl very markedly decreases the survival of isolated CSCs that are in a quiescent state, notably of CSCs isolated from glioblastomas.

### Compound 2

The dihydrolysed compound of bisacodyl (4,4′-dihydroxy-diphenyl-(2-pyridyl)-methane (DDPM)) is commercially available and it was also synthesized in the context of the present study (see example 1 compound 2 (GSC-002)).

This compound (of commercial origin and resynthesized) was tested on the survival of the cancer stem cells TG1 and OB1 in proliferation and in quiescence, on the survival of U-87 MG cells, on the survival of fetal neural stem cells and on the survival of human astrocytes in the same conditions as those used for bisacodyl (compound 1) and using the same ATP-Glo test (Examples 2.4).

Compound 2 (GSC-002) has an effect comparable to bisacodyl (compound 1) on the 5 cellular types tested (FIGS. 4a, b, c, d and e). Its effect on proliferating cancer stem cells seems a little more marked than that of bisacodyl (compound 1).

### Example 11

### Effects of Analogues of DDPM/Compound 2 (GSC-002) on TG1 Cancer Stem Cells and f-NSC Human Fetal Neural Stem Cells

Various analogues were synthesized in order to evaluate the structure-activity relation, see Example 1.

They are the following compounds:
4-((4-Hydroxyphenyl)(pyridin-2-yl)methyl)phenyl acetate (Compound 3 (GSC-006))
(Pyridin-2-ylmethylene)bis(4,1-phenylene)bis(2,2,2-trifluoroacetate) (Compound 5 (GSC-012))
4-((4-Methoxyphenyl)(pyridin-2-yl)methyl)phenol (Compound 6 (GSC-018))
4-((4-Methoxyphenyl)(pyridin-2-yl)methyl)phenyl acetate (Compound 7 (GSC-019))
4-((4-(Prop-2-yn-1-yloxy)phenyl)(pyridin-2-yl)methyl)phenol (Compound 9 (GSC-028))

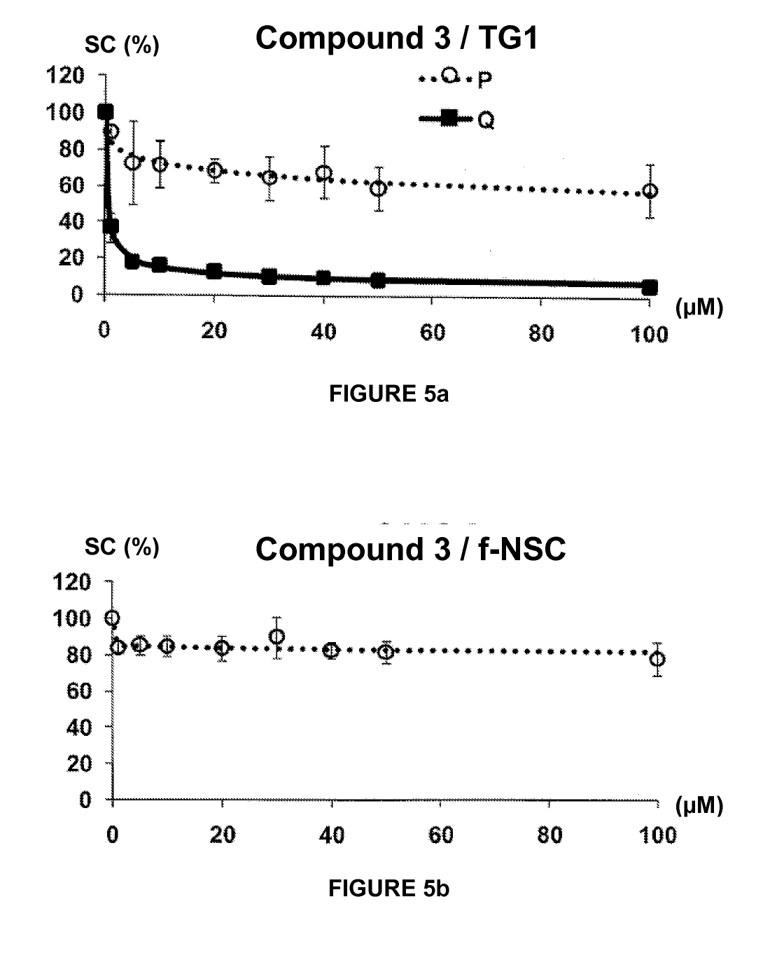

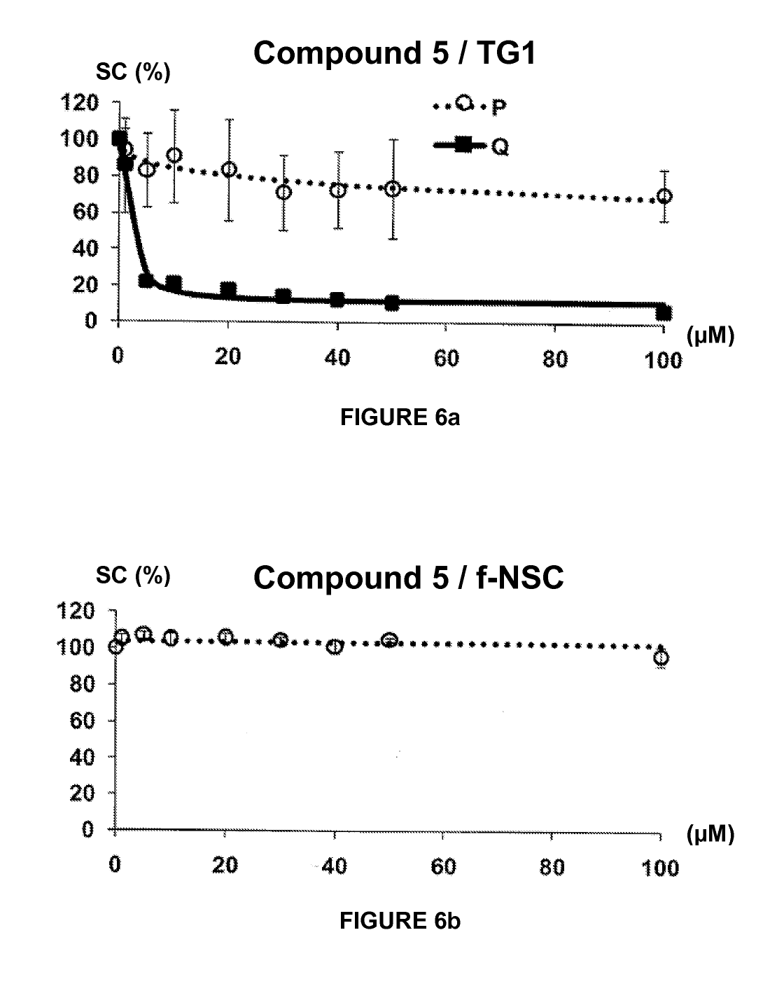

Compounds 3 (GSC-06), 5 (GSC-012), 6 (GSC-018), 7 (GSC-019) showed an effect similar to that described for bisacodyl (compound 1) and the dihydrolysed compound (DDPM/compound 2 (GSC-002)). These molecules showed little or no effect on survival of fetal neural stem cells (fNSC) (FIGS. 5a,b, 6a,b, 7a,b and 8a,b).

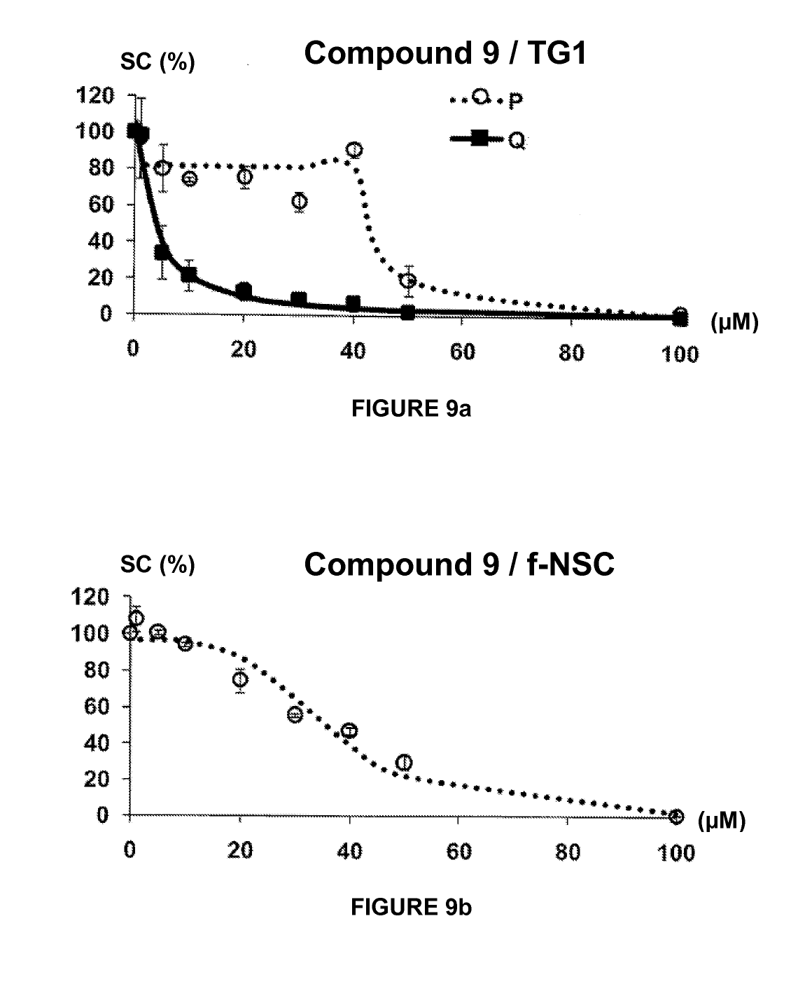

It was noted that compound 9 (GSC-028) is active on the quiescent cells, but also on the proliferating cells. In fact compound 9 (GSC-028) caused a considerable decrease in survival of proliferating cells at concentrations above 40 μM. Moreover, it was noticed that the compound also causes a large decrease in survival of NSC cells starting from 40 μM, although the effect is less cooperative than that observed for the proliferating TG1 cells (FIGS. 9a and b).

### CONCLUSION

Taken together, these results show that bisacodyl and its analogues possessing the same pharmacophore are extremely promising candidates for treating tumours having CSCs capable of entering quiescence, notably glioblastomas.

### Example 12

### High Throughput Screening Identifies Bisacodyl as a Potent Cytotoxic Compound Towards Glioblastoma Stem Cells in an Acidic pH Environment

With the aim of tracking chemical compounds featuring the above mentioned properties, we screened the Prestwick Chemical Library composed of 1180 compounds of known toxicity, on human glioblastoma stem cells. Toxicity of the compounds was evaluated both on proliferating cells and under conditions that favor the quiescent state. Most hit compounds were active under both conditions. One molecule, bisacodyl, showed the greatest specificity towards cells grown under conditions favoring quiescence. Further investigation of the factors that sustain the activity of this compound, pointed to the acidity of the medium. Bisacodyl thus appears as a particularly attractive compound as it acts, in vitro, on proliferating and quiescent glioblastoma stem cells, solely under the acidic conditions at pH values in the range of those found within tumors.

### Materials and Methods

### Materials

Bisacodyl and DDPM were purchased from Sigma-Aldrich. The compound may alternatively be purchased from Ambinter, or otherwise been prepared according to known synthetic methods.

### Cell Culture

Glioblastoma (WHO grade IV glioma) stem cells (TG1, TG16 and OB1) were derived from tumor samples of 3 patients at Sainte Anne Hospital (Paris, France), as previously described (48) and expanded as neurosphere cultures. In continuously proliferating cultures, neurospheres were mechanically dissociated into a single-cell suspension twice a week with 90% renewal of their culture medium. Quiescent glioblastoma stem cells were obtained by non-renewal of the culture medium for 9-16 days following cell seeding. The non-proliferating state was assessed by decreased ability of the cells to incorporate EdU (5-ethynyl-2′-deoxyuridine) using the Click-iT™ EdU Flow Cytometry Assay Kit (Invitrogen, France) according to the manufacturer's instructions.

The glioblastoma U-87 MG cell line was purchased from ATCC and expanded in Eagle's Minimum Essential Medium (EMEM) (ATCC) supplemented with 10% fetal bovine serum (FBS) (Invitrogen, France) and 1% penicillin/streptomycin (Invitrogen, France).

Human fetal neural stem cells were isolated as previously described (49) and kindly provided by Dr Junier (INSERM UMR894, Paris, France). They were cultured as floating spheres in “Neurocult”® NSC Basal Medium (StemCell Technologies) supplemented with NeuroCult® Proliferation Supplement (StemCell Technologies), 10 ng/ml of bFGF and 20 ng/ml of EGF (both from Peprotech, Rocky Hill, N.J.) and mechanically dissociated in a single-cell suspension once every two weeks with 90% renewal of their culture medium.

Primary human astrocytes (HA cells) were expanded in AM Medium (both from ScienCell Research Laboratories, Carlsbad Calif.) according to the manufacturer's instructions.

Human Embryonic Kidney 293 cells (HEK 293 cells) were expanded in Minimum Essential Medium (MEM) with 2 mM L-Glutamine, 100 UI/ml-100 μg/ml Penicillin/Streptomycin and 10% FBS.

Master and working cell banks were established for all cell types and cells were used at defined ranges of cell passages.

### Primary and Secondary Chemical Screens

Chemical screening was performed at the Integrated Chemical Biology Platform (PCBIS; UMS 3286) in Strasbourg, France. Proliferating or quiescent TG1 glioblastoma stem cells were seeded in 50 μl of their respective media containing, respectively, 30 000 and 40 000 cells per well into 96-well opaque bottom plates (Greiner, Courtaboeuf, France) using the Biomek FX robot (Beckman Coulter). 50 μl of each compound (100 μM, 2% DMSO in culture medium) of the Prestwick Chemical Library (Prestwick Chemical, Illkirch, France) were added to the cells. The final concentration of chemical compounds in each well was 50 μM (1% DMSO) and, in the primary screen, each molecule was tested once. Negative control wells (12/96 on each assay plate) contained cells treated with 1% DMSO and positive control wells (4/96 on each assay plate) contained cells treated with 50 μM of the calmodulin inhibitor Ophiobolin A (Sigma Aldrich, Lyon, France). Assay plates were incubated for 24 hours at 37%, 5% CO₂. Cell viability was then assayed using the CellTiter Glo reagent (Promega) according to the manufacturer's instructions. Luminescence, reflecting the amount of ATP in the cells in each well was measured with the Victor™3 multilabel plate reader (PerkinElmer).

Prior to hit selection, positive and negative control wells were used to calculate the Z′ factor evaluating the signal to noise ratio and data dispersion which reflect the quality of the assay (50). The results were taken into account only if Z′ was above 0.5.

Cell viability in each well was determined by calculating the percentage of luminescent signal in the well with respect to the average signal measured in negative control wells (cells treated only with 1% DMSO). A chemical compound was considered as a hit if the ATP level (expressed as % of untreated control under the same conditions) in the respective well was less than 5% and/or if the corresponding luminescent signal was lower than the mean signal of negative control wells minus 5 times the standard deviation from this value.

Primary screen hit compounds were further tested in duplicate and at two different concentrations (50 μM and 5 μM) in a secondary round of screening on proliferating and quiescent TG1 cells. Cell plating and compound treatment conditions were as described for the primary screen. Hit selection/confirmation criteria for quiescent cells were as described above. Due to a higher variability observed on assay plates of proliferating cells, compounds were considered as hits if the corresponding luminescent signal was lower than the mean signal of negative control wells minus only 3 times the standard deviation from this value.

### Hit Validation and EC50 Calculations

Confirmed hit compounds from the primary and secondary screens were purchased from Prestwick Chemical and dissolved in DMSO to obtain 10 mM concentrated stock solutions. Hit compound validation was obtained through dose-response curves on the viability of TG1 glioblastoma stem cells under the same experimental conditions (cell density, treatment duration) as described for the primary and secondary screens. Each compound was tested in triplicate in at least three independent experiments on proliferating and quiescent glioblastoma stem TG1 cells.

Dose response curves and EC50 calculations for compounds identified as being bioactive on TG1 cells, were performed, under the same conditions, on proliferating and quiescent TG16 and OB1 cells. Compound selectivity was evaluated by similar experiments performed on normal fetal neural stem cells (100 000 cells per well), U87-MG glioblastoma cells, HEK293 cells and primary human astrocytes (50 000 cells per well for the last three cell types).

### Curve Fitting for EC50 Calculations

The EC50 value was determined for each compound by fitting the data points according to the following equation:

γ=(S↓max+S↓min x [symbol] (1/ [symbol] EC [symbol] ↓50) [symbol] ↑n)/(1+ [symbol] (1/ [symbol] EC [symbol] ↓50) [symbol] ↑n)

where y represents the expected response (expressed as a percentage of cell viability), x is the chemical compound concentration, Sₘₐₓ and Sₘᵢₙ are the maximum and minimum viability responses recorded, respectively, and n is the Hill coefficient. Curve fitting was performed using the Microsoft Excel Solver component.

### Stability Measurements

10 μM solutions of Bisacodyl and DDPM were prepared in proliferating or quiescent glioblastoma stem cell culture medium in the presence of 1% DMSO. Following protein precipitation (with acetonitrile), samples were analyzed by HPLC after 0, 2, 4, 6 and 24 h of incubation at 37° C.

### Effect of the Culture Medium on Lead Compound Activity

Proliferating TG1 cells were pelleted through centrifugation (220 g) and suspended in conditioned medium from quiescent TG1 cells after 9 days in culture. Conversely, quiescent TG1 cells were suspended in freshly prepared medium. Cells were then seeded into 96-well opaque bottom plates (Greiner, Courtaboeuf, France) (30 000 and 40 000 cells/well, respectively; volume per well: 50 μl). 50 μl of chemical compound solutions at various concentrations (in triplicate) were added and the plates were incubated at 37° C., 5% CO₂ for 24 h. Cell viability was then assayed using the CellTiter-Glo viability assay (Promega). Proliferating TG1 cells in freshly prepared culture medium and quiescent TG1 cells in their usual conditioned medium were treated in the same experimental conditions and used as controls.

### Effect of pH on Lead Compound Activity

HEPES and BisTris buffers were added to the freshly prepared TG1 cell culture medium to a final concentration of 20 mM. The pH of the medium solution was then adjusted to values varying between 7.4 and 6.0 with 1M HCl or a sodium acetate buffer (0.1 M pH=4). The culture media were placed in a CO₂ cell culture incubator settled to 5% CO₂ and 37° C. for 1 hour. The pH was measured again and re-adjusted to the expected values if necessary and culture media were filter-sterilized (0.22 μm filter) prior to their use. The toxicity of DDPM (10 or 100 μM) on proliferating and quiescent TG1 cells was then assessed under the same conditions as those described in the previous section. pH dependency of lead compounds was also assessed for the glioblastoma U-87 MG cell line and on normal fetal neural stem cells and primary human astrocytes in their respective culture media (adjusted to pH 7.4 or 6.2) with HCl (1M) or a sodium acetate buffer (0.1 M pH=4).

### Apoptosis Measurements

Proliferating (30 000 cells/well) and quiescent (40 000 cells/well) TG1 cells were suspended in freshly prepared culture medium (pH=7.3) or acidified freshly prepared culture medium (pH=6.4) and treated with increasing concentrations of the lead compounds for 24 hours. Following treatment, apoptosis was measured with the Apo-ONE Homogeneous Caspase-3/7 Assay from Promega according to the manufacturer's instructions. Staurosporine (Sigma, Aldrich) and culture medium were used as positive and negative controls, respectively.

### Results

### Quiescence of Glioblatoma Stem Cells In Vitro

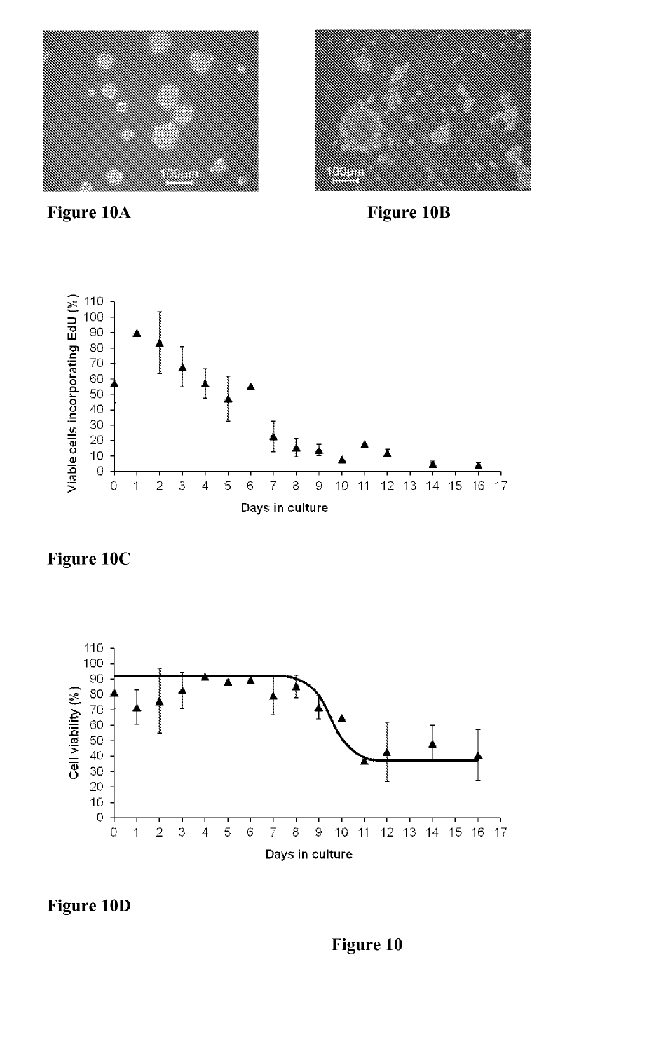

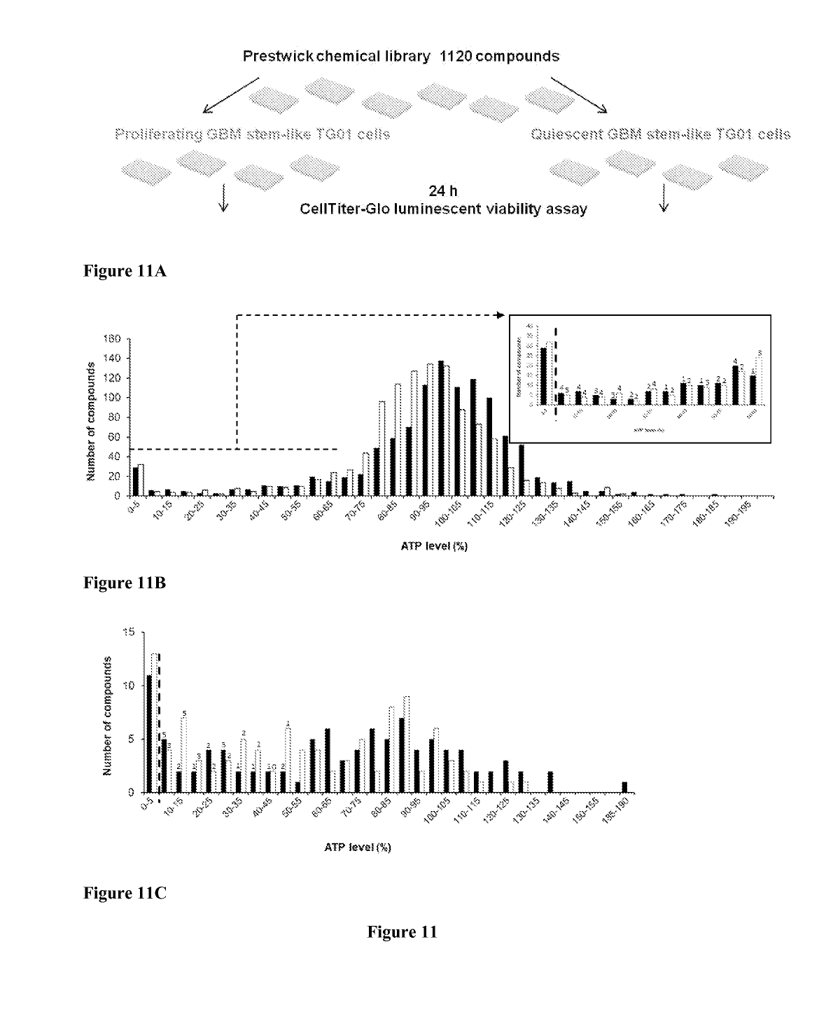

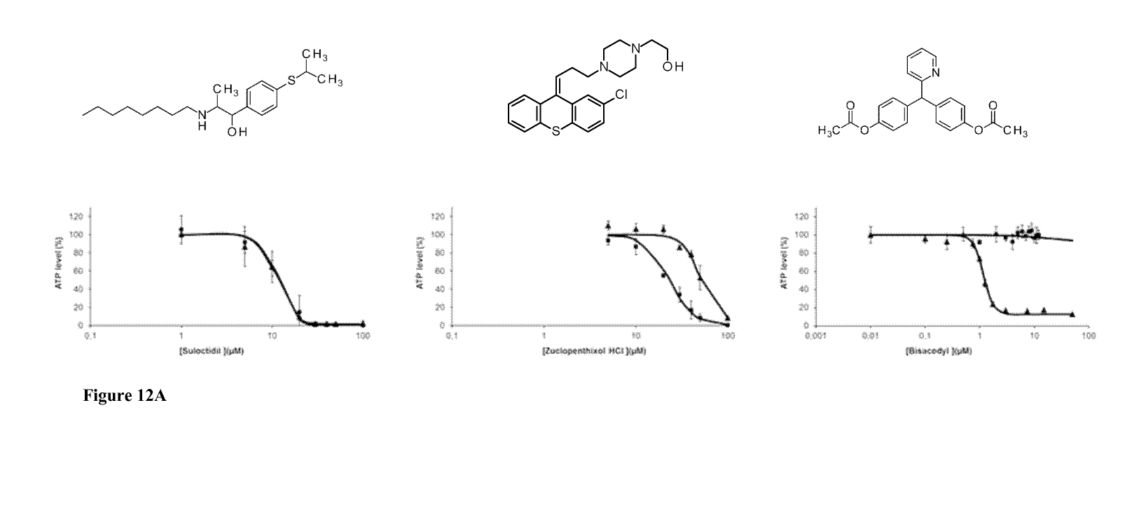

Proliferating glioblastoma stem cells designed as TG1 cells (FIG. 10A) were previously selected, expanded in culture through the neurosphere assay and extensively characterized for their long-term self-renewal and clonal properties as well as for their ability to initiate tumor formation in vivo (48). As mentioned previously, tumor stem cells are likely to persist within the tumor bulk in vivo in a slow-cycling state and this relative quiescence was designed as one of the mechanisms underlying their resistance to current chemotherapeutic agents (38). To achieve quiescence of TG1 cells in vitro, proliferating glioblastoma stem cells were seeded as described in the experimental section (day 0) and left without culture medium renewal for 16 days. From day 1 to day 16, EdU (5-ethyl-2′ deoxyuridine) incorporation was assayed to determine the average proliferating activity of the cells and 7-AAD (7-aminoactinomycine D) staining was used to assess cell viability at each time point. Cell viability data were taken into account for the calculation of the percentage of cells incorporating EdU during the 24 hours of the experiment. As shown in FIG. 10B, glioblastoma stem cells maintained in culture for 16 days without medium renewal are morphologically similar to their proliferating counterparts although the neurospheres appear less numerous and more loose. At day 0, just after cell passaging and during the 24 hours of the experiment, 50-60% of the cells are able to incorporate EdU. The percentage of cells going through the S phase increases significantly at day 1 and 2 and returns to initial levels (50-60% of the cells) by day 4 in culture (FIG. 11C). No significant variations are observed from day 4 to day 6 whereas a marked decrease is observed at day 7. The percentage of cells incorporating EdU reaches very low levels by day 8 and remains at similar values (approximately 15% or less of proliferating cells) until day 16 (FIG. 10C). Cell viability measurements at the same time points indicate that, until day 9, cell viability does not seem to be significantly affected whereas the number of cells incorporating EdU at this time point is markedly affected (see FIGS. 10C and D). At later time points the number of viable cells decreases drastically (FIG. 10D). Finally, we have shown that viable cells after 16 days in these culture conditions are able to re-enter the cell cycle and proliferate again following medium renewal (data not shown). Altogether, these data indicate that quiescent non-proliferating (or at least slow growing) but viable glioblastoma stem cells can be obtained in vitro following growth factor deprivation for at least 8 days. Based on these results, quiescent glioblastoma stem cells were used for further experiments either at 9 or at 16 days after culture medium change.

Identification of Compounds with Toxicity Towards Proliferating and/or Quiescent Glioblastoma Stem Cells by a High-Throughput Screening Approach

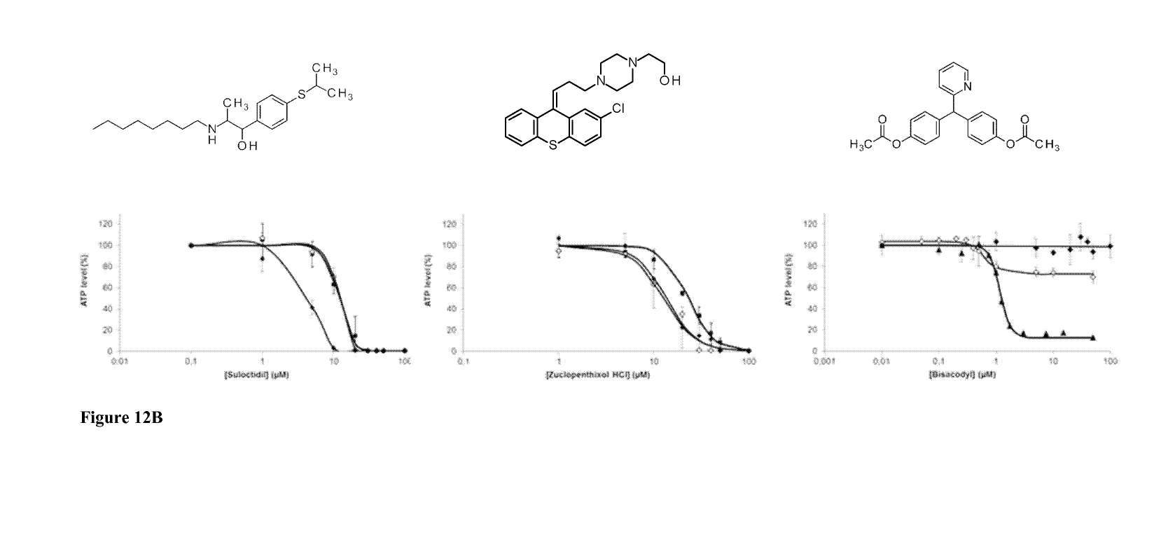

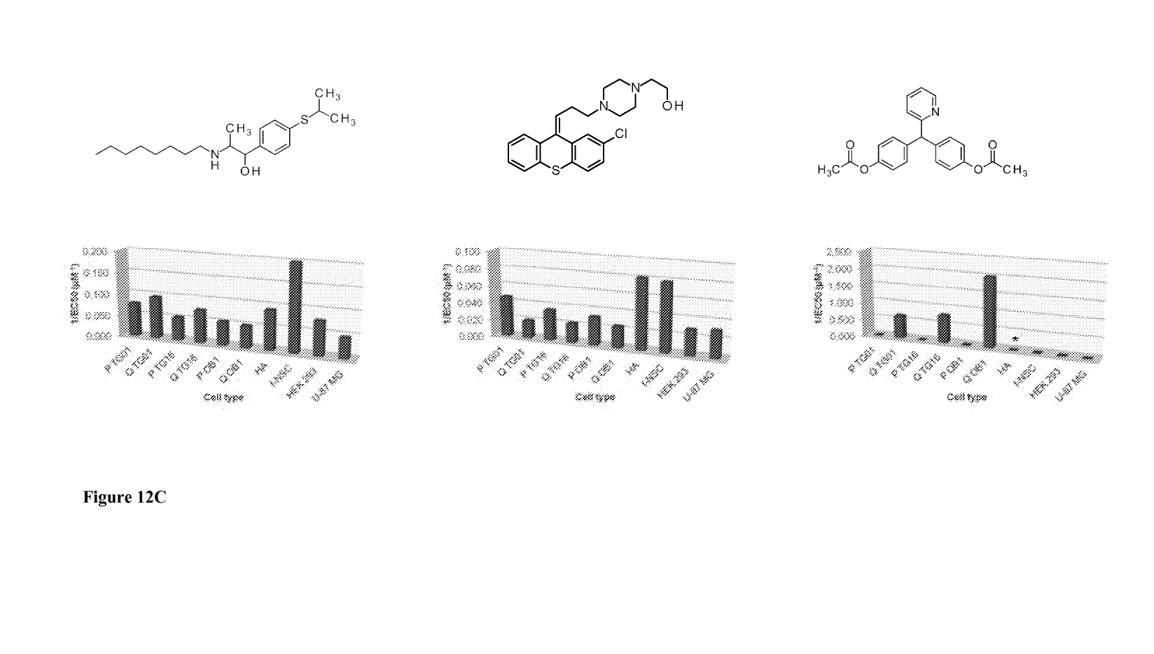

The Prestwick chemical library was screened on TG1 glioblastoma stem cells, with the aim of finding chemical compounds with known toxicity in man and able to interfere with chemo- and radio-resistant glioblastoma cells even in their non-proliferative state. The Prestwick Chemical Library is a commercial collection of 1180 small molecules. Most of the molecules in the library are either marketed drugs or at least drugs that have undergone a phase I clinical trial. According to the manufacturer, the molecules were chosen for their high chemical and pharmacological diversity and their known toxicity and bioavailability in humans. In the primary screen, TG1 cells, grown under proliferative or quiescent conditions were challenged with the different compounds of the library at a 50 μM final concentration. Their ATP-level, which was correlated to cell viability, was measured after 24 hours (FIG. 11A and Experimental section). As indicated under Materials and Methods, hits were selected for their ability to reduce ATP levels significantly (to less than 5% that of the negative controls and/or with a luminescent signal decrease corresponding to a least 5 times the standard deviation measured for these negative controls. As shown in FIG. 12B, in the first screen, about 5% of the test compounds reduce significantly the metabolic activity of glioblastoma stem cells grown under proliferative or quiescent conditions. Of the 1180 compounds, 57 are active on proliferating cells and 69 on quiescent cells), with 40 compounds exhibiting a similar effect in either of the two conditions Seventeen compounds appeared to trigger an increase of ATP levels (FIG. 11B and data not shown). To confirm hits from the primary screen, the 86 compounds which reduced ATP levels and 16/17 molecules that increased ATP levels were retested, in duplicate, at two concentrations (50 μM and 5 μM) on the cells under the two culture conditions. Due to a higher variability observed for the assay plates of proliferating cells, hits of the secondary screen for these cells were selected based on their ability i) to reduce ATP levels to less than 5% of the ones observed in control wells as previously and/or ii) to produce viability signal that was lower than the mean signal of negative control wells minus only 3 times the standard deviation from this value.

The secondary screen confirmed the activity of approximately 50% of the compounds that lowered ATP levels (29/57 compounds for cells under proliferative conditions and 33/69 for cells grown under quiescent conditions) (FIG. 11C). 23 of the confirmed hits are active on both proliferating and quiescent glioblastoma stem cells. Of the 16 compounds tested for the potential to increase the glioblastoma cells ATP level, only one compound was confirmed (FIG. 11C).

The reliability of the primary and the secondary screen was evaluated by calculating the Z′ factor (50) for each assay plate. The median Z′ factor was of 0.615 for the primary screen and of 0.68 for the secondary screen Results from a plate were taken into account only if the Z′ value was higher than 0.5.

Subsequently, dose-response curves were generated on both proliferating and quiescent glioblastoma stem cells (TG1 cells) for 27 out of the 39 active compounds selected in the primary and secondary screens. Representative results of the activity profiles of selected molecules are shown on FIG. 12A. Suloctidil (left panel) was representative of compounds showing overlapping cytotoxic activity towards both proliferating and quiescent glioblastoma stem cells. Other selected hits, including Zuclopenthixol HCl (middle panel) and bisacodyl (right panel) had more selective effects on proliferating or quiescent glioblastoma stem cells, respectively (FIG. 12A). Dose-response curve results were used to determine the effective concentration needed to reduce cell viability by 50% (EC₅₀) (see Experimental section). Table 2 summarizes the results obtained for the 24 compounds that retain activity either on proliferating and/or on quiescent TG1 glioblastoma stem cells.

The activity of these 24 molecules were further tested on two other glioblastoma stem cell types (TG16 and OB1) derived, under conditions similar to those used for TG1 cells, from tumors of two distinct patients. Dose-response curves were also established on both proliferating and quiescent cells. The activity profiles of the 24 selected compounds were similar to the ones observed for TG1 glioblastoma stem cells (FIG. 12C and Table 3).

The selectivity and potency of each selected compound towards glioblastoma stem cells was then assayed by performing dose-response curves on normal human primary astrocytes, normal human fetal neural stem cells, the HEK293 human embryonic kidney cell line and the U87 MG glioblastoma cell line. The majority of the 24 compounds are cytotoxic for all the cell types tested (FIGS. 12B and C, Table 2 and data not shown); Suloctidil (left panel) and Zuclopenthixol HCl (middle panel) are presented as examples of this cytotoxicity. Nevertheless, one molecule, bisacodyl showed a unique activity profile, as its cytotoxicity seems to occur specifically under conditions that trigger glioblastoma stem cells' quiescence. Bisacodyl showed no or little activity on control cell types (FIGS. 12B and C and Table 2). In addition, the compound exhibited highly potent cytotoxic activity on glioblastoma-stem like cells, cultured under quiescent conditions, with an EC₅₀ of 1 μM (FIGS. 12B and C, Table 2). Trypan blue and 7-AAD staining were associated to the ATP-Glo assay to confirm that the ATP level decrease induced by bisacodyl is related to glioblastoma stem cell death and does not correspond to a change in the ATP metabolism of the cells. Under the experimental conditions tested, no staining was observed for HA cells treated with bisacodyl suggesting that the observed decrease in the ATP level (FIG. 12B) was related to metabolic changes and not to cell death (data not shown).

Altogether, these data, pointed out bisacodyl as a highly potent and selective inhibitor of glioblastoma stem cell survival. Due to this unique activity profile (selectivity and potency towards glioblastoma stem cells), Bisacodyl([4-[(4-acetyloxyphenyl)-pyridin-2-ylmethyl]phenyl]acetate) was chosen for further investigation.

In the Culture Medium, Bisacodyl is Hydrolyzed into DDPM, its Known Active Metabolite.

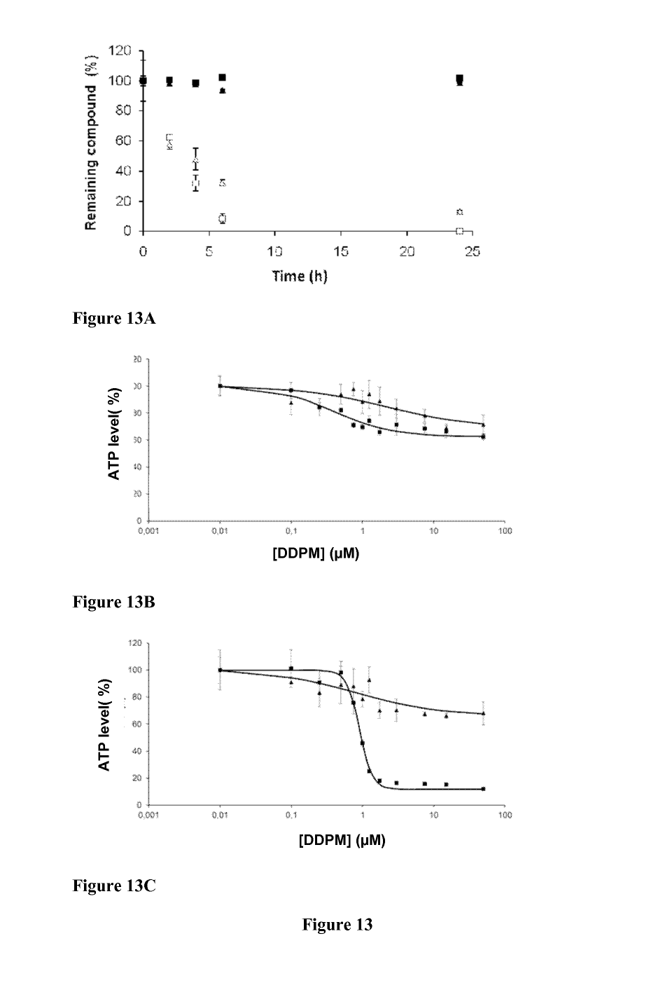

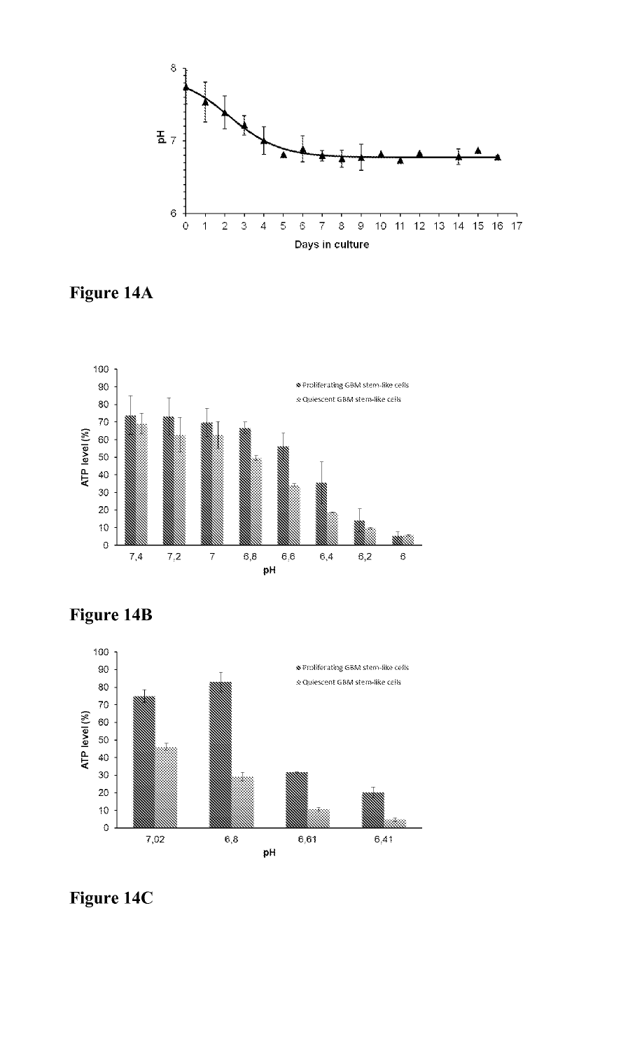

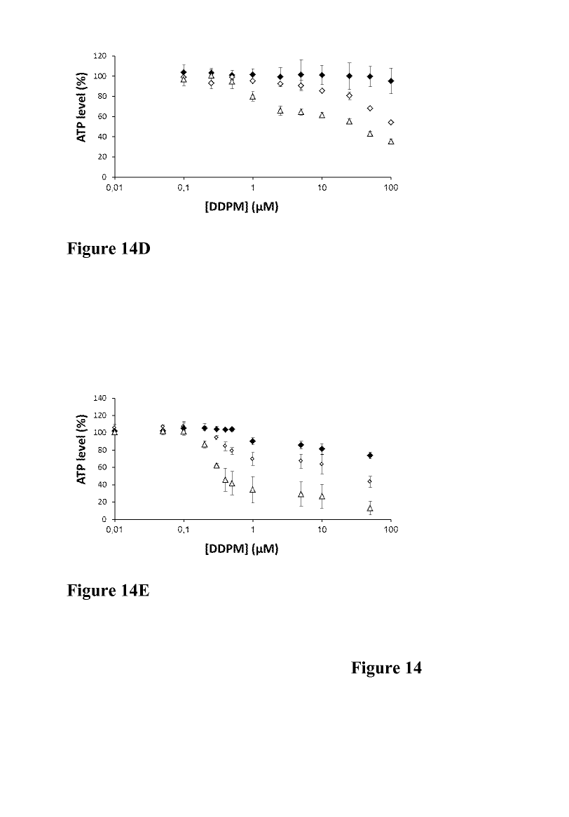

Bisacodyl, like most of the test compounds of the Prestwick Chemical Library used in our screen assay, is a marketed drug currently used as a stimulant laxative for the treatment of constipation and for bowel evacuation before examination procedures in surgery. Bisacodyl is known as a pro-drug which is rapidly converted to the active metabolite 4,4′(dihydroxy-diphenyl)(2-pyridyl)methane (DDPM) (Reynolds, 1993). To test whether biascodyl could be hydrolyzed in vitro under the cell culture conditions, we analyzed its stability in proliferating and quiescent glioblastoma stem cell culture medium. As shown in FIG. 13A, the amount of bisacodyl (expressed as a percentage of the initial concentration) decreases rapidly in both culture media (half-life of approximately 2 hours). No change in DDPM concentration was observed in similar conditions pointing to a stability of the compound in the culture media. Furthermore, using HPLC, we identified DDPM as being the final metabolite of bisacodyl in the culture medium. The effect of DDPM on proliferating and quiescent glioblastoma stem cell viability was then tested. Results presented in FIGS. 14B and 14C indicate that DDPM has the same activity profile as bisacodyl, with a minor effect on proliferating cells (FIG. 13B), and a high cytotoxicity on quiescent glioblastoma stem cells (FIG. 13C). Also, the EC₅₀ value obtained for DDPM is comparable to the one of bisacodyl (EC₅₀≈1 μM). These results reinforce the idea that bisacodyl's action on quiescent glioblastoma stem cells is mediated by DDPM. As a consequence, DDPM, instead of bisacodyl, was used in subsequent studies.

### Influence of the Culture Medium on the Activity of DDPM

Like bisacodyl, DDPM shows preferential activity on quiescent versus proliferating glioblastoma stem cells. We thus asked whether this activity profile was related to the cell status (proliferating versus quiescent) or whether a component present in the conditioned culture medium of quiescent cells could play a role. To answer this question, the effect of DDPM on TG1 glioblatoma cells was measured after media exchange i.e. proliferating glioblastoma stem cells were put into contact with the quiescent cell conditioned medium (9 days in culture without medium renewal) on one hand, and quiescent cells were put into contact with freshly prepared proliferating cell culture medium. DDPM was added for 24-hours and cell survival was measured. The results obtained under these conditions were compared to the effect of DDPM on proliferating and quiescent cells treated in their respective culture media. As shown in FIGS. 13B and C, DDPM has a more pronounced cytotoxic activity on proliferating TG1 cells when these cells are treated in conditioned quiescent cell medium whereas, its effect on quiescent cells is significantly reduced when the treatment is performed in freshly prepared non-conditioned medium. Altogether, these results suggested that the cytotoxic activity of DDPM (and of bisacodyl) on glioblastoma stem cells involves at least one component present in the culture medium of the cells and that it does not only rely on their quiescent state.

### The Cytotoxic Activity of DDPM is pH Dependent

Although multiple differences may exist between the freshly prepared proliferating glioblastoma stem cell medium and conditioned medium from these cells after 9 days in culture, an evident dissimilarity is their pH. Indeed, the pH of freshly prepared medium is close to the value of physiological pH (≈7.7) (see FIG. 14A). When cells are left in culture, without medium renewal, the pH decreases progressively and reaches a value of approximately 6.7 after 9 days (FIG. 14A). Thus, to test whether the pH of the culture medium could influence the cytotoxic activity of DDPM, proliferating and quiescent TG1 cells were treated with this compound at 10 μM for 24 h in freshly prepared culture medium set at pH values varying from 7.4 to 6.0. As shown in FIG. 14B, DDPM is cytotoxic to quiescent TG1 cells at slightly acidic conditions whereas little or no effect of this compound is observed at pH values above 7. Survival of proliferating TG1 cells to DDPM treatment is also decreased in the acidic medium. Nevertheless, quiescent TG1 cells are still more sensitive to DDPM compared to proliferating cells. Similar experiments were performed on TG1 cells in the presence of 100 μM of DDPM under conditions where the acidification of the culture medium was performed with a sodium acetate buffer instead of HCl as in FIG. 14B (see Materials and Methods). As shown in FIG. 14C, under these conditions, the effect of DDPM is more pronounced both on proliferating and quiescent cells compared to the effect of the same compound following acidification of the medium with HCl. Altogether these data suggested that the cytotoxic activity of DDPM on glioblastoma stem cells is pH dependent and that the quiescent state and/or the presence of acetate potentiate the effect this compound.

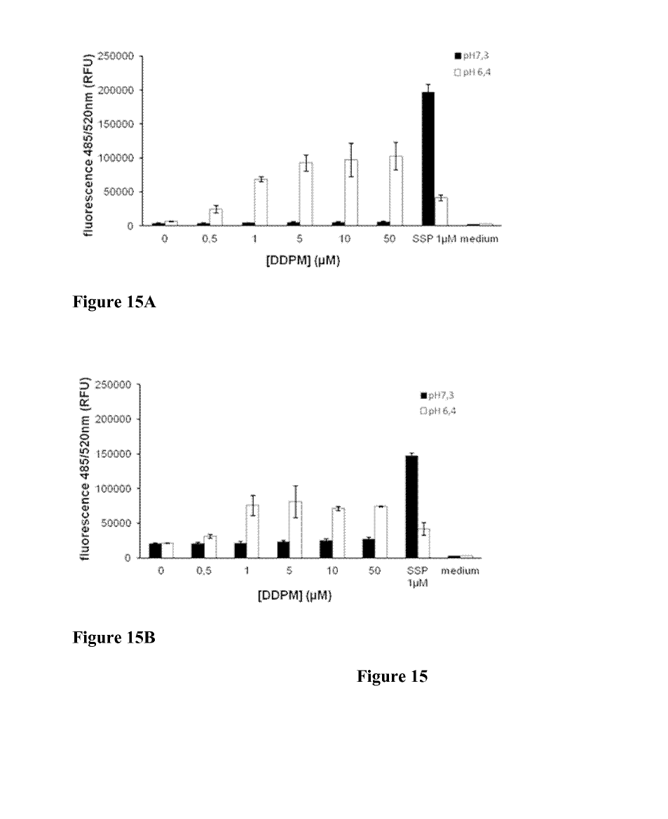

In order to determine whether at a pH around 6.2, DDPM showed specificity towards glioblastoma stem cells compared to other cell types, the cell viability assay was performed on U-87 MG glioblastoma cells, primary human astrocytes (HA cells) and human fetal neural stem cells (f-NSC cells) treated for 24 h with this compound in their respective culture media at a pH set to 6.2. Similar assays were performed on these cell types at physiological pH. The results of these experiments are shown on FIGS. 14D and 15E. DDPM had no cytotoxic effect on U-87 MG glioblastoma cells at physiological pH whereas this effect was significantly higher at pH 6.2 when the pH was set with a 1M HCl solution. The percentage of U-87 cells cell death was even higher in acidic conditions when the pH was adjusted with a sodium acetate buffer (FIG. 14D). Cell death was also assessed using trypan blue staining A pH dependent reduction of ATP levels, related to cell death (observed by trypan blue staining), was also observed for human astrocytes, and this effect was more pronounced in the presence of acetate (FIG. 14E). For f-NSC, acidification of the culture medium was toxic per se (90% of cell death at pH 6.6) even in the absence of DDPM (data not shown).

Altogether, these data first point to a difference of sensitivity among the cell types investigated. Cancer cells and astrocytes are resistant to acidification whereas f-NSC are not. Second, at acidic pH, DDPM is active on all the cell-types tested that survive the acidic extracellular conditions, with a greater sensitivity when the experiments were performed in the presence of acetate

DDPM Stimulates Apoptotic Pathways in Glioblastoma Stem Cells in a pH-Dependent Manner

Bisacodyl's active metabolite DDPM has cytotoxic effects on glioblastoma stem cells. In order to determine if this compound stimulates apoptotic pathways in these cells, we measured the activity of the apoptotic effectors caspase 3 and 7 in proliferating and quiescent glioblastoma stem cells as a function of increasing concentrations of DDPM at two distinct pH values, one close to physiological pH value and the second at a more acidic value (pH=6.4) where DDPM was shown to be the most active. As shown in FIGS. 15A and 15B, at pH=7.3 caspase 3/7 activity was low in proliferating TG1 cells, and slightly higher in quiescent cells. Acidification of the medium does not change caspase activity in either of the two states. Addition of DDPM stimulates caspase 3/7 activity in a dose-dependent manner in both proliferating and quiescent TG1 cells but only at pH 6.4. This pro-apoptotic effect of DDPM is not observed at physiological pH (pH 7.3) (FIGS. 15A and B).

### Discussion

The presence within tumors of cells endowed with properties of radio- and chemo-resistance, able to oscillate between quiescent and proliferating states and to propagate the tumor of origin, shed new light on tumor physiopathology and stressed out the necessity to find new therapeutics and new molecules able to target these cells. Various terms have been coined to cells endowed with these properties (for recent discussion see (51)). As presented in the introduction of this paper, we used the term stem cells for the glioblastoma cells presenting these properties and which were used in the present study.

Screening the Prestwick chemical library for molecules able to interfere with the energy metabolism of these cells and possibly to induce cell death, led to the identification of 24 molecules that were confirmed by dose-response curves. Most molecules were acting on both proliferating and quiescent glioblatoma stem cells. Only 3 showed specificity towards glioblastoma stem cells grown in quiescent conditions, the most specific one being bisacodyl. Further investigation indicated that bisacodyl and its metabolite DDPM exhibited cell toxicity in an acidic pH. Indeed, a gradual increase in cytotoxicity was observed between pH 7 and 6.0, which correspond to pH values at which the tested cells, but f-NSC, were viable.

Glioblastoma is characterized, like many human cancers, by the presence of numerous hypoxic regions. This was attributed to the abnormal and poorly organized tumor vasculature leading to insufficient blood supply (52, 53). There is now extensive evidence indicating that hypoxia plays pivotal roles in tumorigenesis by contributing to increased resistance to radiation and chemotherapy, cell invasion potential and metastasis (53). One of the major phenomena underlying hypoxia induced tumorigenesis is a durable switch to a glycolytic metabolism for hypoxic cells mediated mainly through HIF1, a hypoxia-inducible transcription factor (54) (55). This metabolic switch, which in cancer cells is present event in the absence of hypoxia, results in increased production of lactic and carbonic acids which, when excreted, cause extracellular pH acidification. The acidic environment of tumor cells was also shown to contribute to chemo-resistance by decreasing the cellular uptake of weakly basic drugs or by increasing the activity of ABC-family transporters such as P-glycoprotein (56-59). This property of intratumor microenvironments was also linked to increased tumor invasiveness (60).

Acidic extracellular pH (in the range of 5.6 to 6.8) related to hypoxia, but not exclusively, is a hallmark of intra-tumor microenvironments compared to normal tissue (61) (62) and as such, it is becoming attractive to take advantage of this specificity of tumors for future therapies. Indeed, molecules such as resveratrol and cis-urocanic acid, that show pH-dependent cytotoxic activity towards pancreatic cancer cell lines and human bladder carcinoma cells, respectively, were proposed as new alternatives to cancer treatment (63, 64) and clinical trials with a pro-drug whose proton pump inhibitor activity is revealed in acidic environments are underway (65). Finally, pH-sensitive tumor-targeting nanocarriers are in development (66).

Bisacodyl, and its active metabolite DDPM, affect cell survival at pH values that are found in intratumor microenvironments whereas no cytotoxic activity is observed at physiological pH levels. More importantly, at low pH, these compounds show strong cytotoxic activity not only on glioblastoma derived cell lines but also on cancer stem cells derived from the tumors of three glioblastoma patients and which were shown to resist to TMZ, the actual standard of care for this type of cancer (48). Interestingly, the tumors from which these cancer stem cells have been derived have distinct molecular signatures, indicating, that bisacodyl and DDPM cause cell death through a general mechanism which is not dependent on distinct molecular alterations found in glioblastoma patients. Because glioblastoma stem cells and in general, cancer stem cells are more resistant to conventional treatments compared to the cells of the tumor mass (6) and acquire a more undifferentiated and aggressive phenotype in hypoxic tumor niches (67-70), the ability of bisacodyl to kill cancer cells with stem cell properties only at low pH environments is a major advantage of this compound compared to other pH-sensitive cytotoxic molecules already described. In addition, bisacodyl shows an even higher cytotoxic activity towards slow-cycling “quiescent” glioblastoma stem cells, a cellular state which is favored in hypoxic conditions (71) whereas most chemotherapeutic drugs as well as compounds shown to have pH-sensitive cytotoxic activity target preferentially proliferating cells. To our knowledge, this is the first report of a small molecule with cytotoxic activity towards slow-growing cancer stem cells. Also, in comparison to other molecules targeting tumor cells in their acidic intratumor microenvironment, such as the proton pump inhibitors or urocanic acid, bisacodyl and DDPM exhibit much lower EC₅₀ values.

The origin of tumors, but also the interaction with their environment, their maintenance and propagation are complex and imply contribution of both genetic and epigenetic factors (51). Within a tumor, the state of the cells (or certain cells) may change as a function of their environment, localization, genotoxic and oxidative stresses. Thus, tumor cell plasticity may be another factor rendering tumor eradication a difficult task. Molecules such as bisacodyl, in association with other more conventional therapies appear of invaluable importance as they may allow targeting tumor cells in various possible states or during transition from one state to another.

### Example 13

### Effect of TMZ (Standard of Care for Glioblastoma) and/or DDPM on TG1 Glioblastoma Cancer Stem Cells

Bisacodyl/DDPM decreases the survival of TG1 glioblastoma cancer stem cells, whereas TMZ, the standard of care used in the treatment of glioblastoma is ineffective.

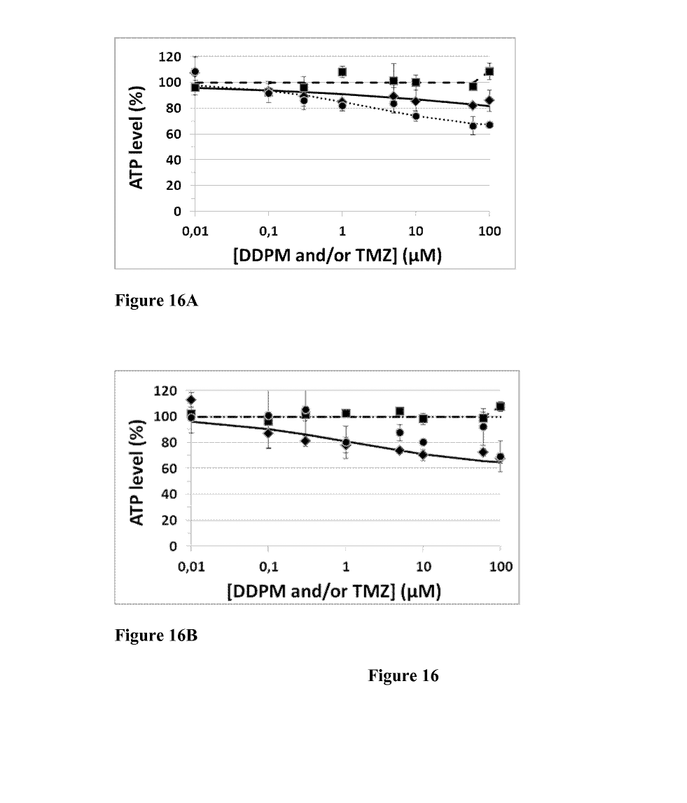

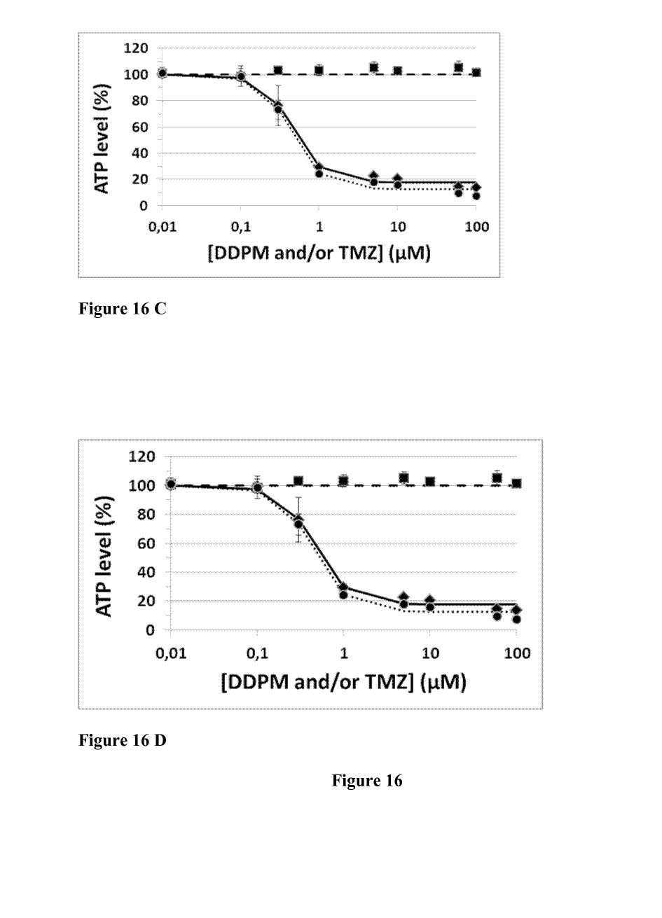

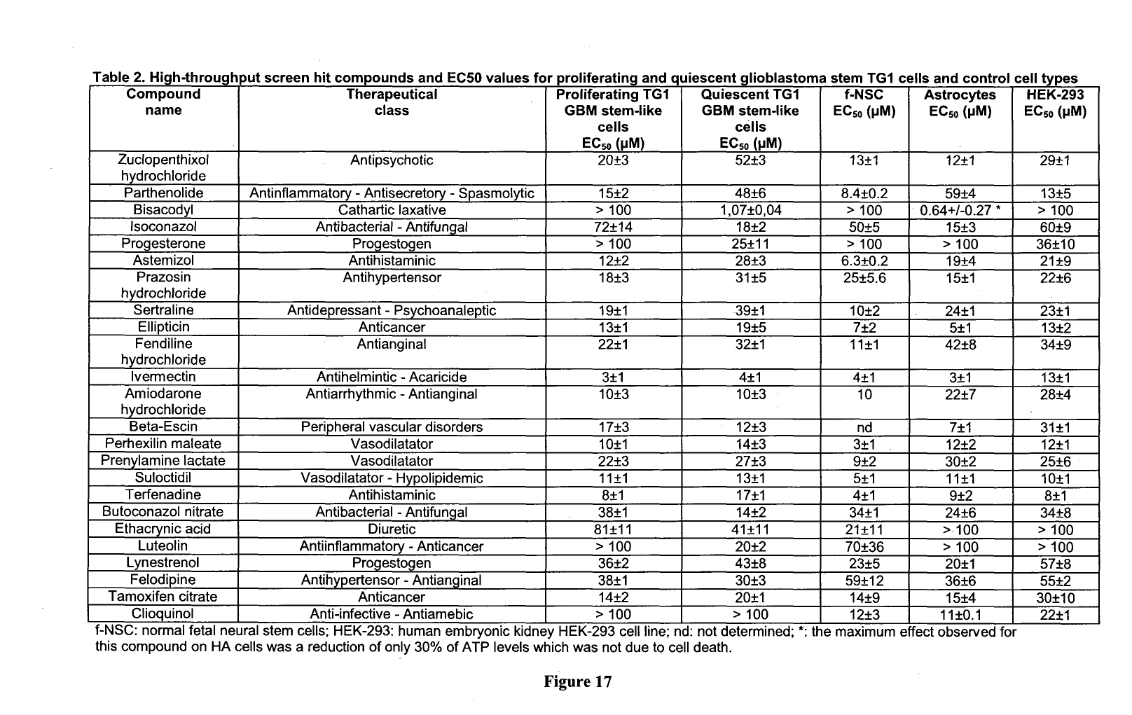

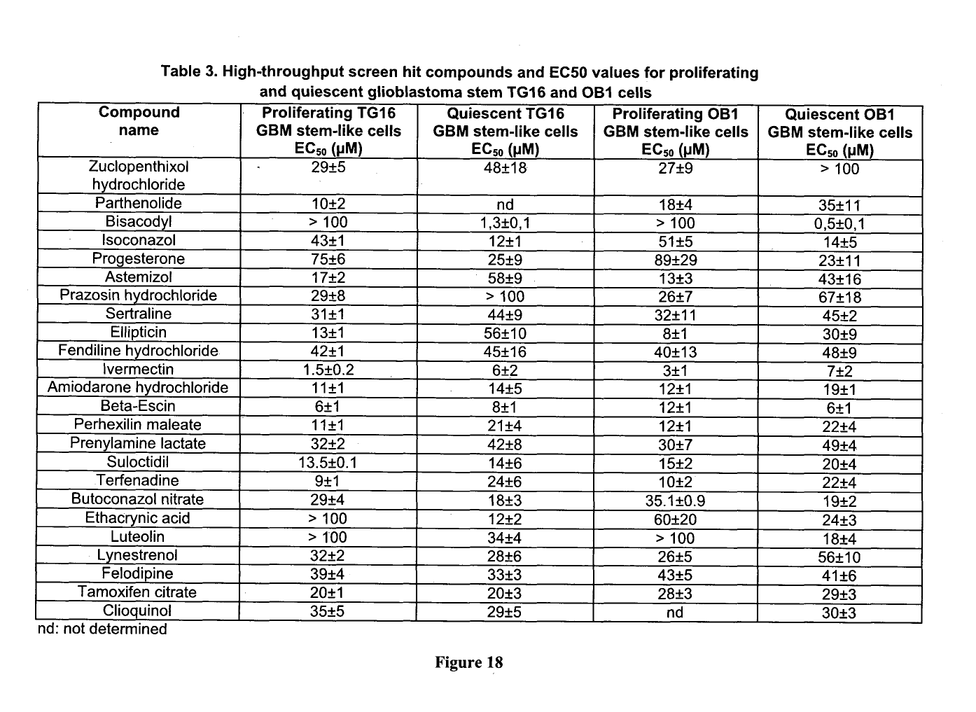

TG1 glioblastoma cancer stem cells were dissociated and cultured in NS34 culture medium at pH 7.35 in the presence of 20 mM Bis-Tris. Cells were exposed to either TMZ (▪), DDPM (♦) and DDPM in the presence of 60 μM TMZ () for 24 hours at 37° C. in the presence of 5% CO2. Cell viability was then assayed using the CellTiter Glo reagent (Promega) according to the manufacturer's instructions. Luminescence, reflecting the amount of ATP in the cells in each well was measured with the Victor™3 multilabel plate reader (PerkinElmer). After 24 hours the level of ATP was measured using the ATP cell titer Glo (Promega). The ATP level expressed as percent compared to untreated cells cultured under the same conditions is reported as a function of TMZ, DDPM and DDPM in the presence of 60 μM TMZ concentrations (FIG. 16A).

TG1 glioblastoma cancer stem cells were dissociated and cultured in NS34 culture medium at pH 7.35 in the presence of 20 mM Bis-Tris. Cells were exposed to either TMZ (▪), DDPM (♦) and DDPM in the presence of 60 μM TMZ () for 72 hours at 37° C. in the presence of 5% CO2. Cell viability was then assayed using the CellTiter Glo reagent (Promega) according to the manufacturer's instructions. Luminescence, reflecting the amount of ATP in the cells in each well was measured with the Victor™3 multilabel plate reader (PerkinElmer). After 72 hours the level of ATP was measured using the ATP cell titer Glo (Promega). The ATP level expressed as percent compared to untreated cells cultured under the same conditions is reported as a function of TMZ, DDPM and DDPM in the presence of 60 μM TMZ concentrations (FIG. 16B).

TG1 glioblastoma cancer stem cells were dissociated and cultured in NS34 culture medium at pH 6.2 in the presence of 20 mM Bis-Tris. Cells were exposed to either TMZ (▪), DDPM (♦) and DDPM in the presence of 60 μM TMZ () for 24 hours at 37° C. in the presence of 5% CO2. Cell viability was then assayed using the CellTiter Glo reagent (Promega) according to the manufacturer's instructions. Luminescence, reflecting the amount of ATP in the cells in each well was measured with the Victor™3 multilabel plate reader (PerkinElmer). After 24 hours the level of ATP was measured using the ATP cell titer Glo (Promega). The ATP level expressed as percent compared to untreated cells cultured under the same conditions is reported as a function of TMZ, DDPM and DDPM in the presence of 60 μM TMZ concentrations. Similar curves were obtained when experiments were performed on TG1 glioblastoma cancer stem cells under quiescent conditions (FIG. 16C). TG1 glioblastoma cancer stem cells were dissociated and cultured in NS34 culture medium at pH 6.2 in the presence of 20 mM Bis-Tris. Cells were exposed to either TMZ (▪), DDPM (♦) and DDPM in the presence of 60 μM TMZ () for 72 hours at 37° C. in the presence of 5% CO2. Cell viability was then assayed using the CellTiter Glo reagent (Promega) according to the manufacturer's instructions. Luminescence, reflecting the amount of ATP in the cells in each well was measured with the Victor™3 multilabel plate reader (PerkinElmer). After 72 hours the level of ATP was measured using the ATP cell titer Glo (Promega). The ATP level expressed as percent compared to untreated cells cultured under the same conditions is reported as a function of TMZ, DDPM and DDPM in the presence of 60 μM TMZ concentrations. Similar curves were obtained when experiments were performed on TG1 glioblastoma cancer stem cells under quiescent conditions (FIG. 16D).

TABLE 4
Effect of tested compounds on TG1-A P and Q, according to the experimental protocol of Example 12

| | EC50 | | EC50 | |
|---|---|---|---|---|
| Structure | Proliferating TG1 | | Quiescent TG1 | |
| 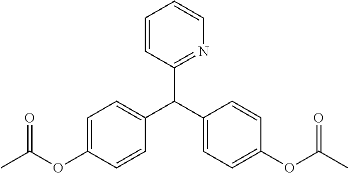 [SMILES: CC(=O)Oc1ccc(C(c2ccc(OC(C)=O)cc2)c2ccccn2)cc1] | >100 | μM | 1.6 ± 0.3 | μM |
| 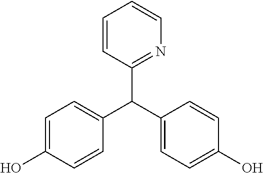 [SMILES: Oc1ccc(C(c2ccc(O)cc2)c2ccccn2)cc1] | >100 | μM | 1.0 ± 0.5 | μM |
| 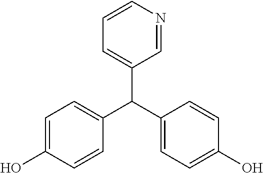 [SMILES: Oc1ccc(C(c2ccc(O)cc2)c2cccnc2)cc1] | >100 | μM | 8 ± 3 | μM |
| 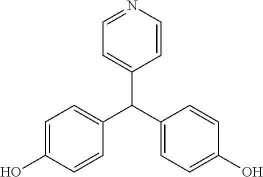 [SMILES: Oc1ccc(C(c2ccncc2)c2ccc(O)cc2)cc1] | >100 | μM | 39 ± 2 | μM |
| 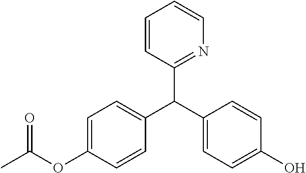 [SMILES: CC(=O)Oc1ccc(C(c2ccc(O)cc2)c2ccccn2)cc1] | >100 | μM | 1.0 ± 0.5 | μM |
| 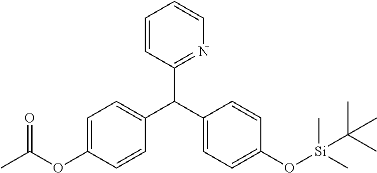 [SMILES: CC(=O)Oc1ccc(C(c2ccc(O[Si](C)(C)C(C)(C)C)cc2)c2ccccn2)cc1] | 12.0 ± 0.1 | μM | 12.1 ± 0.6 | μM |
| 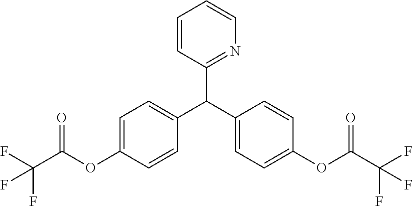 [SMILES: O=C(Oc1ccc(C(c2ccc(OC(=O)C(F)(F)F)cc2)c2ccccn2)cc1)C(F)(F)F] | >100 | μM | 2 ± 1 | μM |
| 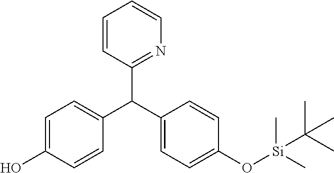 [SMILES: CC(C)(C)[Si](C)(C)Oc1ccc(C(c2ccc(O)cc2)c2ccccn2)cc1] | 13 ± 5 | μM | 13 ± 3 | μM |
| 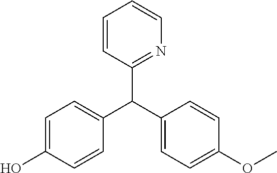 [SMILES: COc1ccc(C(c2ccc(O)cc2)c2ccccn2)cc1] | >100 | μM | 8 ± 6 | μM |
| 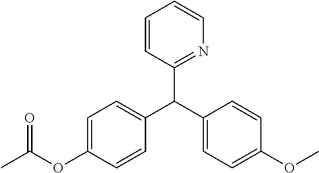 [SMILES: COc1ccc(C(c2ccc(OC(C)=O)cc2)c2ccccn2)cc1] | >100 | μM | 9 ± 4 | μM |
| 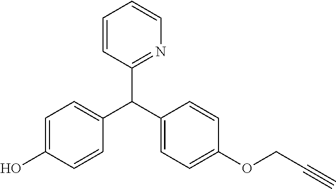 [SMILES: C#CCOc1ccc(C(c2ccc(O)cc2)c2ccccn2)cc1] | 39 ± 6 | μM | 3 ± 1 | μM |
| 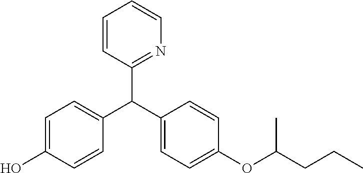 [SMILES: CCCC(C)Oc1ccc(C(c2ccc(O)cc2)c2ccccn2)cc1] | 55 ± 8 | μM | 40 ± 10 | μM |
| 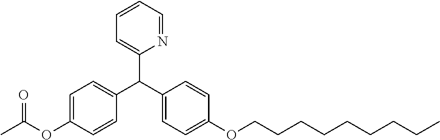 [SMILES: CCCCCCCCCOc1ccc(C(c2ccc(OC(C)=O)cc2)c2ccccn2)cc1] | 25.5 ± 0.6 | μM | 22 ± 2 | μM |
| 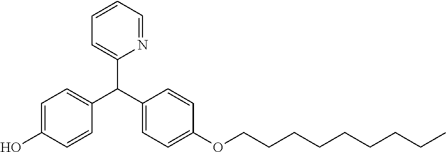 [SMILES: CCCCCCCCCOc1ccc(C(c2ccc(O)cc2)c2ccccn2)cc1] | 25 ± 4 | μM | 22 ± 3 | μM |
| 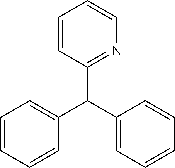 [SMILES: c1ccc(C(c2ccccc2)c2ccccn2)cc1] | >100 | μM | 81.9 | μM |
| 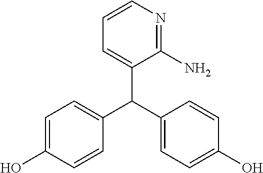 [SMILES: Nc1ncccc1C(c1ccc(O)cc1)c1ccc(O)cc1] | >100 | μM | 32 ± 2 | μM |
| 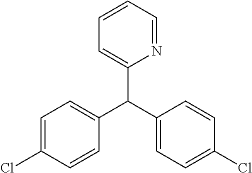 [SMILES: Clc1ccc(C(c2ccc(Cl)cc2)c2ccccn2)cc1] | 34 ± 3 | μM | 35.5 ± 4.5 | μM |
| 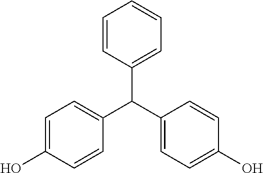 [SMILES: Oc1ccc(C(c2ccccc2)c2ccc(O)cc2)cc1] | 60.8 ± 10.4 | μM | 14.2 ± 8.8 | μM |
| 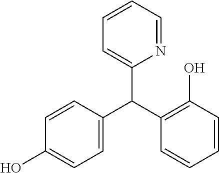 [SMILES: Oc1ccc(C(c2ccccn2)c2ccccc2O)cc1] | >100 | μM | 57.6 ± 0.7 | μM |
| 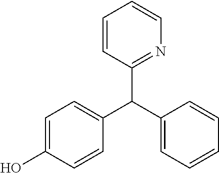 [SMILES: Oc1ccc(C(c2ccccc2)c2ccccn2)cc1] | >100 | μM | 17.1 ± 6.3 | μM |
| 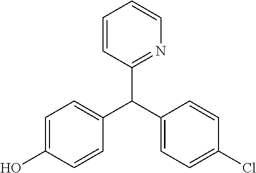 [SMILES: Oc1ccc(C(c2ccc(Cl)cc2)c2ccccn2)cc1] | >100 | μM | 4 ± 1.4 | μM |
| 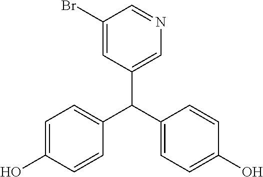 [SMILES: Oc1ccc(C(c2ccc(O)cc2)c2cncc(Br)c2)cc1] | >100 | μM | 5 | μM |
| 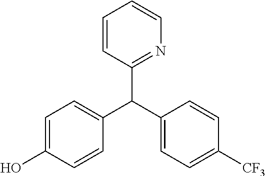 [SMILES: Oc1ccc(C(c2ccc(C(F)(F)F)cc2)c2ccccn2)cc1] | >100 | μM | 17.3 | μM |

### LIST OF REFERENCES

### Indicated Between Brackets in the Text

[1] Patru, C., Romao, L., Varlet, P., Coulombel, L., Raponi, E., Cadusseau, J., Renault-Mihara, F., Thirant, C., Leonard, N., Berhneim, A., Mihalescu-Maingot, M., Haiech, J., Bieche, I., Moura-Neto, V., Daumas Duport, C., Junier, M. P., and Chneiweiss, H. (2010) CD133, CD15/SSEA-1, C034 or side populations do not resume tumor-initiating properties of long-term cultured cancer stem cells from human malignant glio-neuronal tumors. BMC Cancer 10, 66.
[2] Singh, S. K., Clarke, I. D., Terasaki, M., Bonn, V. E., Hawkins, C., Squire, J., and Dirks, P. B. (2003). Identification of a cancer stem cell in human brain tumors. Cancer Res 63, 5821-5828.
[3] Thirant C, Bessette B, Varlet P, Puget S, Cadusseau J, Dos Reis Tavares S, Studler J M, Silvestre D C, Susini A, Villa C, Miguel C, Bogeas A, Surena A L, Dias-Morais A, Léonard N, Pflumio F, Bièche I, Boussin F D, Sainte-Rose C, Grill J, Daumas-Duport C, Chneiweiss H, Junier M P. Clinical relevance of tumor cells with stem-like properties in pediatric brain tumors. PLoS One. 2011 Jan. 28; 6(1):e16375.
[4] Galan-Moya E M, Le Guelte A, Lima-Fernandes E, Thirant C, Dwyer J, Bidere N, Couraud P O, Scott M, Junier M P, Chneiweiss H, Gavard J. Brain endothelial cells maintain glioblastoma stem-like cell expansion through the mTOR pathway. EMBO Report 2011, 12, 470-471.
[5] Silvestre D C, Pineda Marti J R, Hoffschir F, Studler J M, Mouthon M A, Pflumio F, Junier M P, Chneiweiss H, Boussin F D. Alternative Lengthening of Telomeres in Human Glioma Stem Cells. Stem Cells. 2011 Jan. 14.
[6] Schatton, T., Murphy, G. F., Frank, N. Y., Yamaura, K., Waaga-Gasser, A. M., Gasser, M., Zhan, Q., Jordan, S., Duncan, L. M., Weishaupt, C., Fuhlbrigge, R. C., Kupper, T. S., Sayegh, M. H., and Frank, M. H. (2008). Identification of cells initiating human melanomas. Nature 451, 345-349.
[7] Reya, T., Morrison, S. J., Clarke, M. F., and Weissman, I. L. (2001). Stem cells, cancer, and cancer stem cells. Nature 414, 105-111.
[8] Rosen, J. M., and Jordan, C. T. (2009). The increasing complexity of the cancer stem cell paradigm. Science 324, 1670-1673.
[9] European Medicines Agency (2009) “European Public Assessment Report (EPAR) Temodal”—EPAR summary for the public
[10] Stoll, R. E., Blanchard, K. T., Stoltz, J. H., Majeska, J. B., Furst, S., Lilly, P. O., and Mennear, J. H. (2006). Phenolphthalein and bisacodyl: assessment of genotoxic and carcinogenic responses in heterozygous p53 (+/−) mice and syrian hamster embryo (SHE) assay. Toxicol Sci 90, 440-450.
[11] Pala et al. (1968). A New Synthesis of 4,4′-dihydroxydiphenyl-(2-pyridyl)methane. Tetrahedron, 24(2), pp. 619-624.
[12] Al-Hajj, M., Wicha, M. S., Benito-Hernandez, A., Morrison, S. J., and Clarke, M. F. (2003). Prospective identification of tumorigenic breast cancer cells. Proc Natl Acad Sci USA 100, 3983-3988.
[13] Bonnet, D., and Dick, J. E. (1997). Human acute myeloid leukemia is organized as a hierarchy that originates from a primitive hematopoietic cell. Nat Med 3, 730-737.
[14] Chan, K. S., Espinosa, I., Chao, M., Wong, D., Ailles, L., Diehn, M., Gill, H., Presti, J., Jr., Chang, H. Y., van de Rijn, M., Shortliffe, L., and Weissman, I. L. (2009). Identification, molecular characterization, clinical prognosis, and therapeutic targeting of human bladder tumor-initiating cells. Proc Natl Acad Sci USA 106, 14016-14021.
[15] Collins, A. T., Berry, P. A., Hyde, C., Stower, M. J., and Maitland, N. J. (2005). Prospective identification of tumorigenic prostate cancer stem cells. Cancer Res 65, 10946-10951.
[16] Dalerba, P., Dylla, S. J., Park, I. K., Liu, R., Wang, X., Cho, R. W., Hoey, T., Gurney, A., Huang, E. H., Simeone, D. M., Shelton, A. A., Parmiani, G., Castelli, C., and Clarke, M. F. (2007). Phenotypic characterization of human colorectal cancer stem cells. Proc Natl Acad Sci USA 104, 10158-10163.
[17] Eramo, A., Lotti, F., Sette, G., Pilozzi, E., Biffoni, M., Di Virgilio, A., Conticello, C., Ruco, L., Peschle, C., and De Maria, R. (2008). Identification and expansion of the tumorigenic lung cancer stem cell population. Cell Death Differ 15, 504-514.
[18] Fang, D., Nguyen, T. K., Leishear, K., Finko, R., Kulp, A. N., Hotz, S., Van Belle, P. A., Xu, X., Elder, D. E., and Herlyn, M. (2005). A tumorigenic subpopulation with stem cell properties in melanomas. Cancer Res 65, 9328-9337.
[19] Galli, R., Binda, E., Orfanelli, U., Cipelletti, B., Gritti, A., De Vitis, S., Fiocco, R., Foroni, C., Dimeco, F., and Vescovi, A. (2004). Isolation and characterization of tumorigenic, stem-like neural precursors from human glioblastoma. Cancer Res 64, 7011-7021.
[20] Ginestier, C., Hur, M. H., Charafe-Jauffret, E., Monville, F., Dutcher, J., Brown, M., Jacquemier, J., Viens, P., Kleer, C. G., Liu, S., Schott, A., Hayes, D., Birnbaum, D., Wicha, M. S., and Dontu, G. (2007). ALDH1 is a marker of normal and malignant human mammary stem cells and a predictor of poor clinical outcome. Cell Stem Cell 1, 555-567.
[21] Hemmati, H. D., Nakano, I., Lazareff, J. A., Masterman-Smith, M., Geschwind, D. H., Bronner-Fraser, M., and Kornblum, H. I. (2003). Cancerous stem cells can arise from pediatric brain tumors. Proc Natl Acad Sci USA 100, 15178-15183.
[22] Hermann, P. C., Huber, S. L., Herrler, T., Aicher, A., Ellwart, J. W., Guba, M., Bruns, C. J., and Heeschen, C. (2007). Distinct populations of cancer stem cells determine tumor growth and metastatic activity in human pancreatic cancer. Cell Stem Cell 1, 313-323.
[23] Kim, C. F., Jackson, E. L., Woolfenden, A. E., Lawrence, S., Babar, I., Vogel, S., Crowley, D., Bronson, R. T., and Jacks, T. (2005). Identification of bronchioalveolar stem cells in normal lung and lung cancer. Cell 121, 823-835.
[24] Li, C., Heidt, D. G., Dalerba, P., Burant, C. F., Zhang, L., Adsay, V., Wicha, M., Clarke, M. F., and Simeone, D. M. (2007). Identification of pancreatic cancer stem cells. Cancer Res 67, 1030-1037.
[25] Matsui, W., Huff, C. A., Wang, Q., Malehorn, M. T., Barber, J., Tanhehco, Y., Smith, B. D., Civin, C. I., and Jones, R. J. (2004). Characterization of clonogenic multiple myeloma cells. Blood 103, 2332-2336.
[26] O'Brien, C. A., Pollett, A., Gallinger, S., and Dick, J. E. (2007). A human colon cancer cell capable of initiating tumour growth in immunodeficient mice. Nature 445, 106-110.
[27] Prince, M. E., Sivanandan, R., Kaczorowski, A., Wolf, G. T., Kaplan, M. J., Dalerba, P., Weissman, I. L., Clarke, M. F., and Ailles, L. E. (2007). Identification of a subpopulation of cells with cancer stem cell properties in head and neck squamous cell carcinoma. Proc Natl Acad Sci USA 104, 973-978.
[28] Quintana, E., Shackleton, M., Sabel, M. S., Fullen, D. R., Johnson, T. M., and Morrison, S. J. (2008). Efficient tumour formation by single human melanoma cells. Nature 456, 593-598.
[29] Ricci-Vitiani, L., Lombardi, D. G., Pilozzi, E., Biffoni, M., Todaro, M., Peschle, C., and De Maria, R. (2007). Identification and expansion of human colon-cancer-initiating cells. Nature 445, 111-115.
[30] Schatton, T., Murphy, G. F., Frank, N. Y., Yamaura, K., Waaga-Gasser, A. M., Gasser, M., Zhan, Q., Jordan, S., Duncan, L. M., Weishaupt, C., Fuhlbrigge, R. C., Kupper, T. S., Sayegh, M. H., and Frank, M. H. (2008). Identification of cells initiating human melanomas. Nature 451, 345-349.
[31] Singh, S. K., Hawkins, C., Clarke, I. D., Squire, J. A., Bayani, J., Hide, T., Henkelman, R. M., Cusimano, M. D., and Dirks, P. B. (2004). Identification of human brain tumour initiating cells. Nature 432, 396-401.
[32] Szotek, P. P., Pieretti-Vanmarcke, R., Masiakos, P. T., Dinulescu, D. M., Connolly, D., Foster, R., Dombkowski, D., Preffer, F., Maclaughlin, D. T., and Donahoe, P. K. (2006). Ovarian cancer side population defines cells with stem cell-like characteristics and Mullerian Inhibiting Substance responsiveness. Proc Natl Acad Sci USA 103, 11154-11159.
[33] Taylor, M. D., Poppleton, H., Fuller, C., Su, X., Liu, Y., Jensen, P., Magdaleno, S., Dalton, J., Calabrese, C., Board, J., Macdonald, T., Rutka, J., Guha, A., Gajjar, A., Curran, T., and Gilbertson, R. J. (2005). Radial glia cells are candidate stem cells of ependymoma. Cancer Cell 8, 323-335.
[34] Yang, Z. F., Ho, D. W., Ng, M. N., Lau, C. K., Yu, W. C., Ngai, P., Chu, P. W., Lam, C. T., Poon, R. T., and Fan, S. T. (2008). Significance of CD90+ cancer stem cells in human liver cancer. Cancer Cell 13, 153-166.
[35] Yuan, X., Curtin, J., Xiong, Y., Liu, G., Waschsmann-Hogiu, S., Farkas, D. L., Black, K. L., and Yu, J. S. (2004). Isolation of cancer stem cells from adult glioblastoma multiforme. Oncogene 23, 9392-9400.
[36] Zhang, S., Balch, C., Chan, M. W., Lai, H. C., Matei, D., Schilder, J. M., Yan, P. S., Huang, T. H., and Nephew, K. P. (2008). Identification and characterization of ovarian cancer-initiating cells from primary human tumors. Cancer Res 68, 4311-4320.

### OTHER REFERENCES

### Indicated Between Parentheses in the Text

1. Furnari F B, Fenton T, Bachoo R M, et al. Malignant astrocytic glioma: genetics, biology, and paths to treatment. Genes Dev 2007; 21(21):2683-710.
2. Stupp R, Mason W P, van den Bent M J, et al. Radiotherapy plus concomitant and adjuvant temozolomide for glioblastoma. N Engl J Med 2005; 352(10):987-96.
3. Vescovi A L, Galli R, Reynolds B A. Brain tumour stem cells. Nat Rev Cancer 2006; 6(6):425-36.
4. Hanahan D, Weinberg R A. Hallmarks of cancer: the next generation. Cell 2011; 144(5):646-74.
5. Frank N Y, Schatton T, Frank M H. The therapeutic promise of the cancer stem cell concept. J Clin Invest 2011; 120(1):41-50.
6. Sengupta A, Cancelas J A. Cancer stem cells: a stride towards cancer cure? J Cell Physiol 2010; 225(1):7-14.
7. Bonnet D, Dick J E. Human acute myeloid leukemia is organized as a hierarchy that originates from a primitive hematopoietic cell. Nat Med 1997; 3(7):730-7.
8. Lapidot T, Sirard C, Vormoor J, et al. A cell initiating human acute myeloid leukaemia after transplantation into SCID mice. Nature 1994; 367(6464):645-8.
9. Galli R, Binda E, Orfanelli U, et al. Isolation and characterization of tumorigenic, stem-like neural precursors from human glioblastoma. Cancer Res 2004; 64(19):7011-21.
10. Hemmati H D, Nakano I, Lazareff J A, et al. Cancerous stem cells can arise from pediatric brain tumors. Proc Natl Acad Sci USA 2003; 100(25):15178-83.
11. Singh S K, Clarke I D, Terasaki M, et al. Identification of a cancer stem cell in human brain tumors. Cancer Res 2003; 63(18):5821-8.
12. Singh S K, Hawkins C, Clarke I D, et al. Identification of human brain tumour initiating cells. Nature 2004; 432(7015):396-401.
13. Yuan X, Curtin J, Xiong Y, et al. Isolation of cancer stem cells from adult glioblastoma multiforme. Oncogene 2004; 23(58):9392-400.
14. Al-Hajj M, Wicha M S, Benito-Hernandez A, Morrison S J, Clarke M F. Prospective identification of tumorigenic breast cancer cells. Proc Natl Acad Sci USA 2003; 100(7):3983-8.
15. Dalerba P, Dylla S J, Park I K, et al. Phenotypic characterization of human colorectal cancer stem cells. Proc Natl Acad Sci USA 2007; 104(24):10158-63.
16. O'Brien C A, Pollett A, Gallinger S, Dick J E. A human colon cancer cell capable of initiating tumour growth in immunodeficient mice. Nature 2007; 445(7123):106-10.
17. Ricci-Vitiani L, Lombardi D G, Pilozzi E, et al. Identification and expansion of human colon-cancer-initiating cells. Nature 2007; 445(7123):111-5.
18. Fang D, Nguyen T K, Leishear K, et al. A tumorigenic subpopulation with stem cell properties in melanomas. Cancer Res 2005; 65(20):9328-37.
19. Schatton T, Murphy G F, Frank N Y, et al. Identification of cells initiating human melanomas. Nature 2008; 451(7176):345-9.
20. Reya T, Morrison S J, Clarke M F, Weissman I L. Stem cells, cancer, and cancer stem cells. Nature 2001; 414(6859):105-11.
21. Bao S, Wu Q, McLendon R E, et al. Glioma stem cells promote radioresistance by preferential activation of the DNA damage response. Nature 2006; 444(7120):756-60.
22. Hirschmann-Jax C, Foster A E, Wulf G G, et al. A distinct “side population” of cells with high drug efflux capacity in human tumor cells. Proc Natl Acad Sci USA 2004; 101(39):14228-33.
23. Liu G, Yuan X, Zeng Z, et al. Analysis of gene expression and chemoresistance of CD133+ cancer stem cells in glioblastoma. Mol Cancer 2006; 5:67.
24. Capper D, Gaiser T, Hartmann C, et al. Stem-cell-like glioma cells are resistant to TRAIL/Apo2L and exhibit down-regulation of caspase-8 by promoter methylation. Acta Neuropathol 2009; 117(4):445-56.
25. Folkins C, Shaked Y, Man S, et al. Glioma tumor stem-like cells promote tumor angiogenesis and vasculogenesis via vascular endothelial growth factor and stromal-derived factor 1. Cancer Res 2009; 69(18):7243-51.
26. Fan X, Khaki L, Zhu T S, et al. NOTCH pathway blockade depletes CD133-positive glioblastoma cells and inhibits growth of tumor neurospheres and xenografts. Stem Cells; 28(1):5-16.
27. Gallia G L, Tyler B M, Hann C L, et al. Inhibition of Akt inhibits growth of glioblastoma and glioblastoma stem-like cells. Mol Cancer Ther 2009; 8(2):386-93.
28. Mazzoleni S, Politi L S, Pala M, et al. Epidermal growth factor receptor expression identifies functionally and molecularly distinct tumor-initiating cells in human glioblastoma multiforme and is required for gliomagenesis. Cancer Res 2010; 70(19):7500-13.
29. Piccirillo S G, Reynolds B A, Zanetti N, et al. Bone morphogenetic proteins inhibit the tumorigenic potential of human brain tumour-initiating cells. Nature 2006; 444(7120):761-5.
30. Wang J, Wakeman T P, Lathia J D, et al. Notch promotes radioresistance of glioma stem cells. Stem Cells 2010; 28(1):17-28.
31. Bao S, Wu Q, Sathomsumetee S, et al. Stem cell-like glioma cells promote tumor angiogenesis through vascular endothelial growth factor. Cancer Res 2006; 66(16):7843-8.
32. Calabrese C, Poppleton H, Kocak M, et al. A perivascular niche for brain tumor stem cells. Cancer Cell 2007; 11(1):69-82.
33. Folkins C, Man S, Xu P, Shaked Y, Hicklin D J, Kerbel R S. Anticancer therapies combining antiangiogenic and tumor cell cytotoxic effects reduce the tumor stem-like cell fraction in glioma xenograft tumors. Cancer Res 2007; 67(8):3560-4.
34. Fareh M, Turchi L, Virolle V, et al. The miR 302-367 cluster drastically affects self-renewal and infiltration properties of glioma-initiating cells through CXCR4 repression and consequent disruption of the SHH-GLI-NANOG network. Cell Death Differentiation 2012; 19(2):232-44.
35. Gupta P B, Onder T T, Jiang G, et al. Identification of selective inhibitors of cancer stem cells by high-throughput screening. Cell 2009; 138(4):645-59.
36. Pollard S M, Yoshikawa K, Clarke I D, et al. Glioma stem cell lines expanded in adherent culture have tumor-specific phenotypes and are suitable for chemical and genetic screens. Cell Stem Cell 2009; 4(6):568-80.
37. Visnyei K, Onodera H, Damoiseaux R, et al. A molecular screening approach to identify and characterize inhibitors of glioblastoma stem cells. Mol Cancer Ther 2011; 10(10):1818-28.
38. Moore N, Lyle S. Quiescent, slow-cycling stem cell populations in cancer: a review of the evidence and discussion of significance. J Oncol 2011; 2011.
39. Gao M Q, Choi Y P, Kang S, Youn J H, Cho N H. CD24+ cells from hierarchically organized ovarian cancer are enriched in cancer stem cells. Oncogene 2010; 29(18):2672-80.
40. Haraguchi N, Ishii H, Mimori K, et al. CD13 is a therapeutic target in human liver cancer stem cells. J Clin Invest 2010; 120(9):3326-39.
41. Roesch A, Fukunaga-Kalabis M, Schmidt E C, et al. A temporarily distinct subpopulation of slow-cycling melanoma cells is required for continuous tumor growth. Cell 2010; 141(4):583-94.
42. Pece S, Tosoni D, Confalonieri S, et al. Biological and molecular heterogeneity of breast cancers correlates with their cancer stem cell content. Cell; 140(1):62-73.
43. Dembinski J L, Krauss S. Characterization and functional analysis of a slow cycling stem cell-like subpopulation in pancreas adenocarcinoma. Clin Exp Metastasis 2009; 26(7):611-23.
44. Guan Y, Gerhard B, Hogge D E. Detection, isolation, and stimulation of quiescent primitive leukemic progenitor cells from patients with acute myeloid leukemia (AML). Blood 2003; 101(8):3142-9.
45. Holyoake T, Jiang X, Eaves C, Eaves A. Isolation of a highly quiescent subpopulation of primitive leukemic cells in chronic myeloid leukemia. Blood 1999; 94(6):2056-64.
46. Meng S, Tripathy D, Frenkel E P, et al. Circulating tumor cells in patients with breast cancer dormancy. Clin Cancer Res 2004; 10(24):8152-62.
47. Naumov G N, Townson J L, MacDonald I C, et al. Ineffectiveness of doxorubicin treatment on solitary dormant mammary carcinoma cells or late-developing metastases. Breast Cancer Res Treat 2003; 82(3):199-206.
48. Patru C, Romao L, Varlet P, et al. CD133, CD15/SSEA-1, CD34 or side populations do not resume tumor-initiating properties of long-term cultured cancer stem cells from human malignant glio-neuronal tumors. BMC Cancer 2010; 10:66.
49. Thirant C, Bessette B, Varlet P, et al. Clinical relevance of tumor cells with stem-like properties in pediatric brain tumors. PLoS One 2011; 6(1):e16375.
50. Zhang J H, Chung T D, Oldenburg K R. A Simple Statistical Parameter for Use in Evaluation and Validation of High Throughput Screening Assays. J Biomol Screen 1999; 4(2):67-73.
51. Visvader J E. Cells of origin in cancer. Nature 2011; 469(7330):314-22.
52. Bar E E. Glioblastoma, cancer stem cells and hypoxia. Brain Pathol 2011; 21(2):119-29.
53. Jensen R L. Brain tumor hypoxia: tumorigenesis, angiogenesis, imaging, pseudoprogression, and as a therapeutic target. J Neurooncol 2009; 92(3):317-35.
54. Porporato P E, Dhup S, Dadhich R K, Copetti T, Sonveaux P. Anticancer targets in the glycolytic metabolism of tumors: a comprehensive review. Front Pharmacol 2011; 2:49.
55. Chiche J, Brahimi-Horn M C, Pouyssegur J. Tumour hypoxia induces a metabolic shift causing acidosis: a common feature in cancer. J Cell Mol Med; 14(4):771-94.
56. Adams D J. The impact of tumor physiology on camptothecin-based drug development. Curr Med Chem Anticancer Agents 2005; 5(1):1-13.
57. Thews O, Gassner B, Kelleher D K, Schwerdt G, Gekle M. Impact of extracellular acidity on the activity of P-glycoprotein and the cytotoxicity of chemotherapeutic drugs. Neoplasia 2006; 8(2):143-52.
58. Tredan O, Galmarini C M, Patel K, Tannock I F. Drug resistance and the solid tumor microenvironment. J Natl Cancer Inst 2007; 99(19):1441-54.
59. Vukovic V, Tannock I F. Influence of low pH on cytotoxicity of paclitaxel, mitoxantrone and topotecan. Br J Cancer 1997; 75(8):1167-72.
60. Gatenby R A, Gawlinski E T, Gmitro A F, Kaylor B, Gillies R J. Acid-mediated tumor invasion: a multidisciplinary study. Cancer Res 2006; 66(10):5216-23.
61. Lindner D, Raghavan D. Intra-tumoural extra-cellular pH: a useful parameter of response to chemotherapy in syngeneic tumour lines. Br J Cancer 2009; 100(8):1287-91.
62. Marie S K, Shinjo S M. Metabolism and brain cancer. Clinics (Sao Paulo) 2011; 66 Suppl 1:33-43.
63. Peuhu E, Kaunisto A, Laihia J K, Leino L, Eriksson J E. Molecular targets for the protodynamic action of cis-urocanic acid in human bladder carcinoma cells. BMC Cancer 2010; 10:521.
64. Shamim U, Hanif S, Albanyan A, et al. Resveratrol-induced apoptosis is enhanced in low pH environments associated with cancer. J Cell Physiol 2012; 227(4): 1493-500.
65. De Milito A, Canese R, Marino M L, et al. pH-dependent antitumor activity of proton pump inhibitors against human melanoma is mediated by inhibition of tumor acidity. Int J Cancer 2010; 127(1):207-19.
66. Lee E S, Gao Z, Bae Y H. Recent progress in tumor pH targeting nanotechnology. J Control Release 2008; 132(3):164-70.
67. Heddleston J M, Li Z, Lathia J D, Bao S, Hjelmeland A B, Rich J N. Hypoxia inducible factors in cancer stem cells. Br J Cancer 2010; 102(5):789-95.
68. Heddleston J M, Li Z, McLendon R E, Hjelmeland A B, Rich J N. The hypoxic microenvironment maintains glioblastoma stem cells and promotes reprogramming towards a cancer stem cell phenotype. Cell Cycle 2009; 8(20):3274-84.
69. Seidel S, Garvalov B K, Wirta V, et al. A hypoxic niche regulates glioblastoma stem cells through hypoxia inducible factor 2 alpha. Brain 2010; 133 (Pt 4):983-95.
70. Soeda A, Park M, Lee D, et al. Hypoxia promotes expansion of the CD133-positive glioma stem cells through activation of HIF-1alpha. Oncogene 2009; 28(45):3949-59.
71. Ljungkvist A S, Bussink J, Rijken P F, Kaanders J H, van der Kogel A J, Denekamp J. Vascular architecture, hypoxia, and proliferation in first-generation xenografts of human head-and-neck squamous cell carcinomas. Int J Radiat Oncol Biol Phys 2002; 54(1):215-28.
72. Song et al., Cancer Drug Discovery and Development: Cancer Drug resistance”, Chapter 2, pp. 21-42 (2006).
73. Wilkinson, M. C.; Saez, F.; Hon, W. L. Synlett 2006, 7, 1063-1066.
74. Mameri
75. Shi, B-F.; Maugel, N.; Zhang, Y-H. Yu J-Q. Angew. Chem. Int. Ed. 2008, 47, 4882-4886.

1. Compound of the following formula (A): 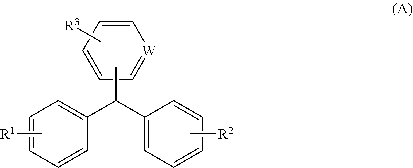
a pharmaceutically acceptable salt thereof, wherein:
R¹ and R² represent independently —H; a linear or branched Ci_{—6} alkyl group —OH; F; CI; Br; I; —NR^{a}R^{b} where R^{a} and R^{b} represent independently H or a linear, branched or cyclic Ci_{—6} alkyl group and where R^{a} and R^{b} can form, together with the nitrogen atom to which they are attached, a heterocycle with 5 or 6 ring members; —OR; —C(0)-NH—R; -0-C(0)-R; —NH—C(0)-R; —NH—S0₂—R; —OSiR°3 where each occurrence of R^{c} represents, independently of the other occurrences of R^{c}, a linear, branched or cyclic Ci_{—6} alkyl group, —OSO₃″;
OPO₃^{2″}; —OSO₃H; —OPθ₃¾; where each occurrence of R represents, independently of the other occurrences of R, a hydrogen atom or an optionally substituted linear, branched or cyclic Ci_{—6} alkyl, C₂_{_{—}}₆ alkene, C₂_{_{—}}₆ alkyne or Ci_{—6} haloalkyl group; and where at least one of the radicals R¹ and R² is different from H;
—W represents C(—R³), N or N⁺(—R⁴) in which R³ represents H; —OH; F; CI; Br; I;
—NR^{a}R^{b} where R^{a} and R^{b} represent independently H or a linear, branched or cyclic Ci_{—}6 alkyl group and where R^{a} and R^{b} can form, together with the nitrogen atom to which they are attached, a heterocycle with 5 or 6 ring members; —OR where R represents an optionally substituted linear, branched or cyclic Ci_{—6} alkyl, C₂_{_{—}}₆ alkene, C₂_{_{—}}₆ alkyne or Ci_{—6} haloalkyl group; or —C(0)OR^{d} where R^{d} represents H or a linear, branched or cyclic Ci_{—6} alkyl group; and R⁴ represents a linear, branched or cyclic Ci_{—6} alkyl group; and
—R⁵ represents a hydrogen atom; a linear or branched Ci_{—6} alkyl group; —OH; F; CI; Br; I; —CF₃; —N0₂; —OR′ wherein R′ represents a hydrogen atom or an optionally substituted linear, branched or cyclic Ci_{—6} alkyl or Ci_{—6} haloalkyl group; or —NR^{c}R^{d} wherein R^{c} et R^{d} independently represent H, a linear, branched or cyclic Ci_{—6} alkyl group, R^{a} et R^{b} and where R^{c} and R^{d} can form, together with the nitrogen atom to which they are attached, a heterocycle with 5 or 6 ring members;
with the proviso that when W is N and R⁵ is hydrogen or methyl, then R¹ and R² are not independently hydrogen, methyl, C1-C3 alkoxy, halogen, nitro, or trifluoromethyl;
for use as a medicinal product in the treatment of cancers containing cancer stem cells and tumour initiating cells.

2. Compound as recited in claim 1, wherein the compound has one of the following structures: 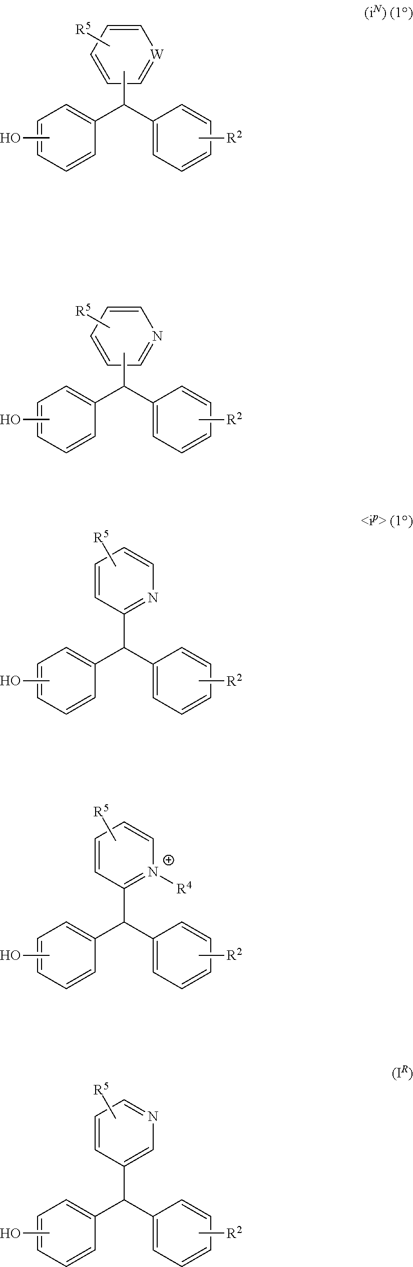 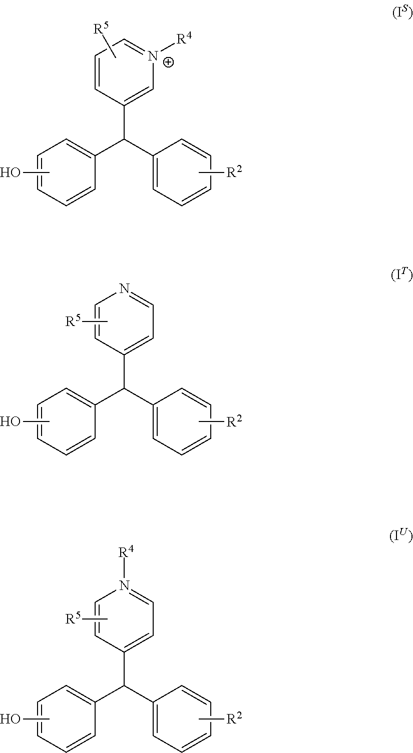
or pharmaceutically acceptable salt thereof;
wherein for each of the structures I^{N} to I^{u}, W, R², R⁴ and R⁵ are as defined in claim 1.

3. Compound as recited in claim 1, wherein the compound has one of the following structures: 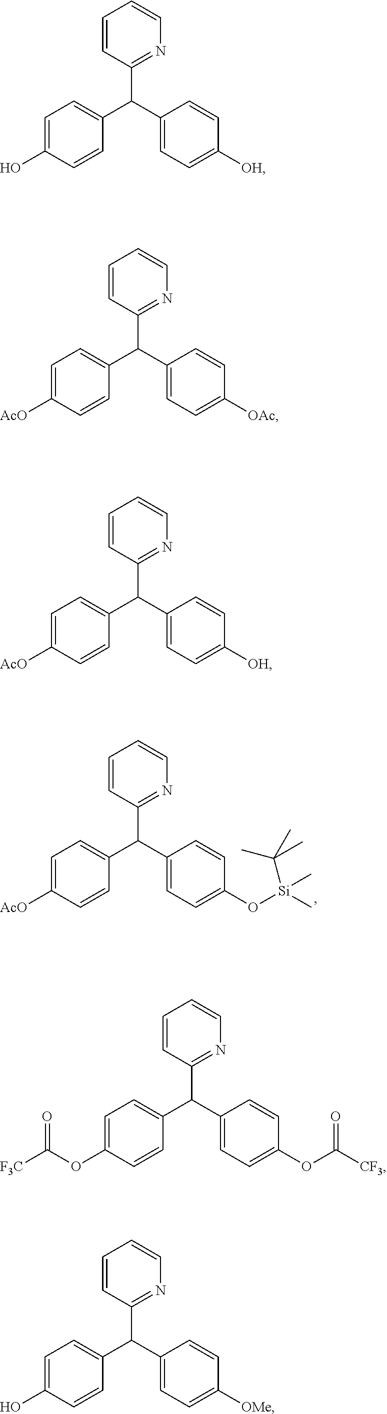 [SMILES: Oc1ccc(C(c2ccc(O)cc2)c2ccccn2)cc1 ; CC(=O)Oc1ccc(C(c2ccc(OC(C)=O)cc2)c2ccccn2)cc1 ; CC(=O)Oc1ccc(C(c2ccc(O)cc2)c2ccccn2)cc1 ; CC(=O)Oc1ccc(C(c2ccc(O[Si](C)(C)C(C)(C)C)cc2)c2ccccn2)cc1 ; O=C(Oc1ccc(C(c2ccc(OC(=O)C(F)(F)F)cc2)c2ccccn2)cc1)C(F)(F)F ; COc1ccc(C(c2ccc(O)cc2)c2ccccn2)cc1] 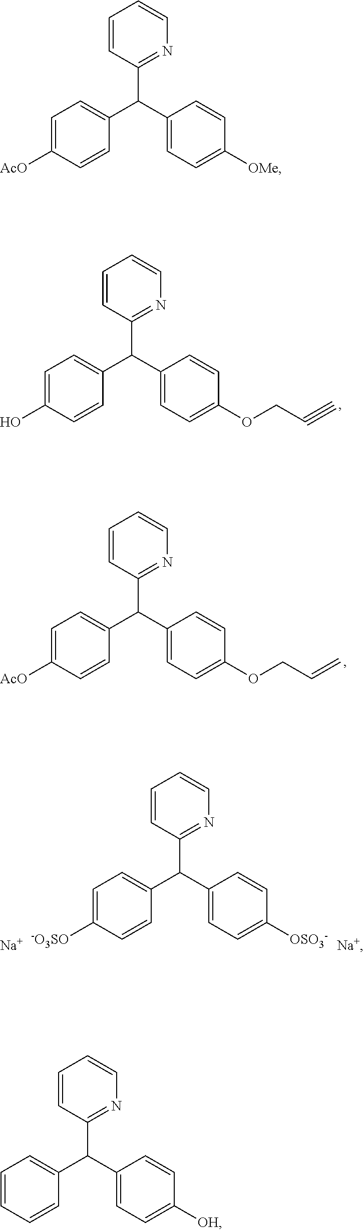 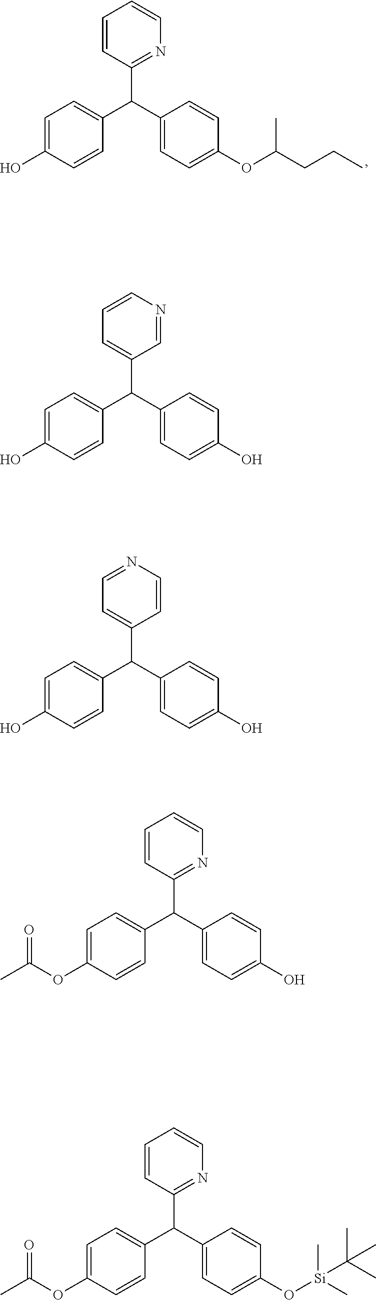 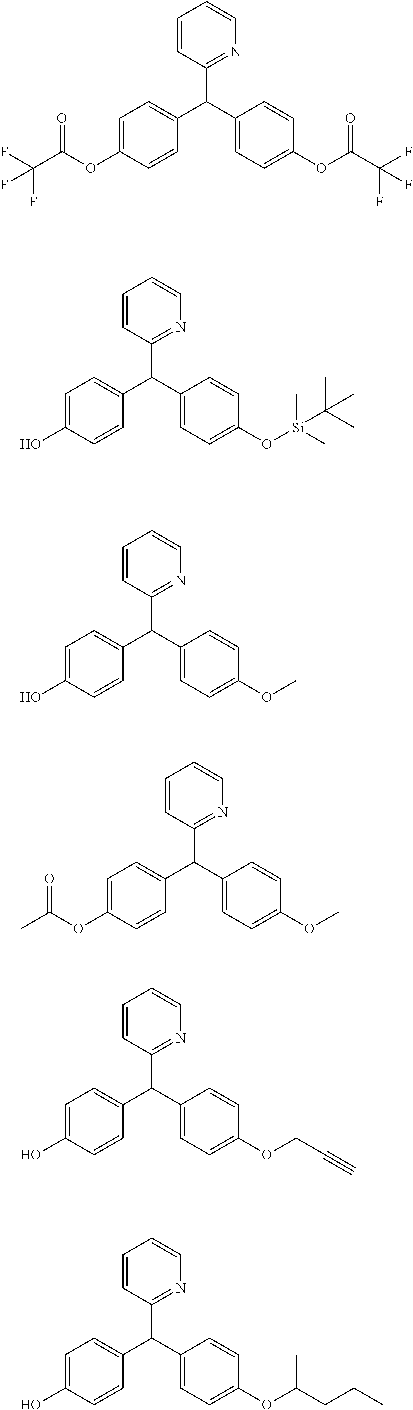 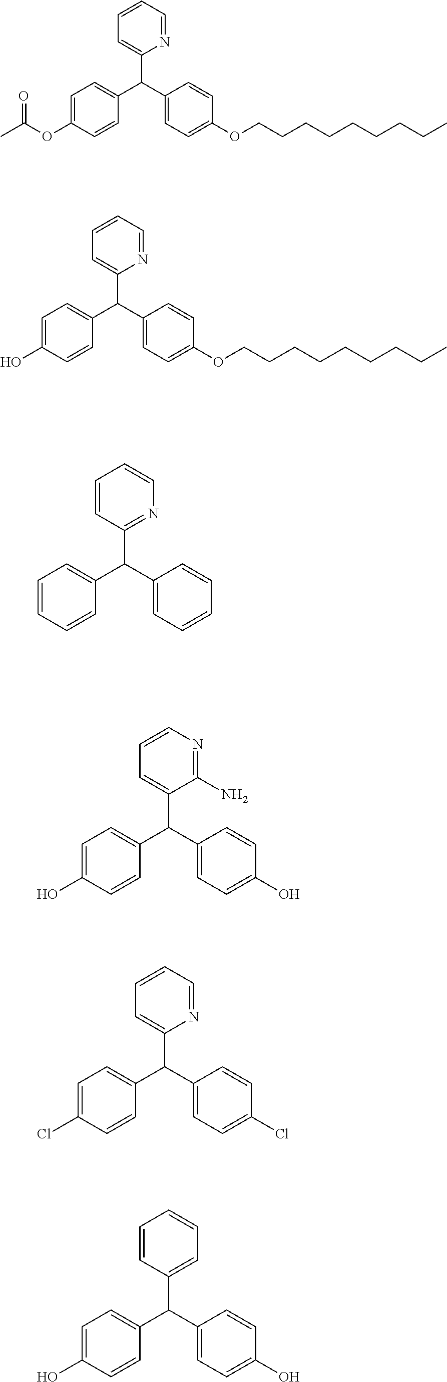 [SMILES: CCCCCCCCCOc1ccc(C(c2ccc(OC(C)=O)cc2)c2ccccn2)cc1 ; CCCCCCCCCOc1ccc(C(c2ccc(O)cc2)c2ccccn2)cc1 ; c1ccc(C(c2ccccc2)c2ccccn2)cc1 ; Nc1ncccc1C(c1ccc(O)cc1)c1ccc(O)cc1 ; Clc1ccc(C(c2ccc(Cl)cc2)c2ccccn2)cc1 ; Oc1ccc(C(c2ccccc2)c2ccc(O)cc2)cc1] 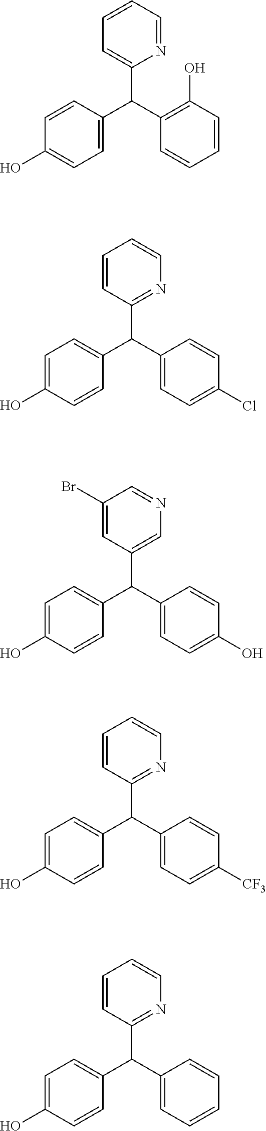 [SMILES: Oc1ccc(C(c2ccccn2)c2ccccc2O)cc1 ; Oc1ccc(C(c2ccc(Cl)cc2)c2ccccn2)cc1 ; Oc1ccc(C(c2ccc(O)cc2)c2cncc(Br)c2)cc1 ; Oc1ccc(C(c2ccc(C(F)(F)F)cc2)c2ccccn2)cc1 ; Oc1ccc(C(c2ccccc2)c2ccccn2)cc1]
or a pharmaceutically acceptable salt thereof.

4. Compound as recited in claim 1, wherein the compound has the following structure: 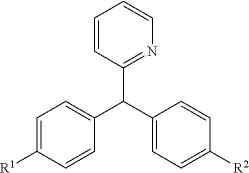
wherein at least one of R¹ or R² represents —OH; an optionally substituted linear, branched or cyclic Ci_{—6}alkyl, C₂_{_{—}}₆alkenyl, C₂_{_{—}}₆alkynyl or Ci_{—6}haloalkyl radical; —OR; -0-C(0)-R; —OSiR°3 wherein each occurrence of R^{c} independently represents a linear, branched or cyclic Ci_{—6}alkyl radical; —OSO₃″; —OPO₃^{2″}; —OSO₃H; —OP03¾; —OP03R₂; wherein each occurrence of R independently represents a hydrogen atom, or an optionally substituted linear, branched or cyclic Ci_{—6}alkyl, C₂_{_{—}}₆alkenyl, C₂_{_{—}}₆alkynyl or Ci_{—6}haloalkyl radical; with the proviso that R¹ and R² may not be independently hydrogen, methyl, C1-C3 alkoxy, halogen, nitro, or trifluoromethyl.

5. Compound as recited in claim 1, wherein the compound has one of the following structures:

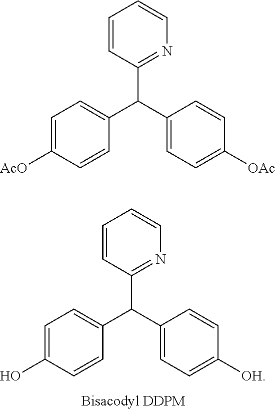 [SMILES: CC(=O)Oc1ccc(C(c2ccc(OC(C)=O)cc2)c2ccccn2)cc1 ; Oc1ccc(C(c2ccc(O)cc2)c2ccccn2)cc1]

6. Compound as recited in claim 1, wherein the compound is in an amount to detectably exhibit cytotoxic activity towards proliferating and/or quiescent cancer stem cells.

7. Compound as recited in claim 1 wherein when said compound possesses cytotoxicity activity towards quiescent cancer stem cells.

8. Compound according to claim 1 for use as a medicinal product intended for treating cancers comprising cancer stem cells and tumour initiating cells present in the group of tumours comprising glioblastomas, melanomas, mammary tumours, tumours of the colon, prostate, kidney, pancreas, lung, or bones.

9. Compound as recited in claim 1, for use in combination with a therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating destructive bone disorders, an agent for treating liver disease, an anti-viral agent, an agent for treating blood disorders, an agent for treating diabetes, or an agent for treating immunodeficiency disorders.

10. Compound as recited in claim 9, wherein the additional therapeutic agent is an anti-proliferative agent.

11. Compound as recited in claim 1 for use in exhibiting cytotoxic activity in proliferating and/or quiescent cancer stem cells.

12. Compound as recited in claim 1 for use in treating primary mammalian tumor sites and/or metastatic sites in a subject.

13. Compound as recited in claim 1 for use in treating chemo- and/or radio-resistant cancer in a subject.

14. Compound as recited in claim 1 for use in preventing or lessening the recurrence of cancer in a subject.

15. Compound as recited in claim 1 for use in treating an aggressive cancer in a subject.

16. Compound as recited in claim 1 for use in preventing cancer in a subject genetically predisposed to cancer, wherein said cancer is associated with cancer stem cells in quiescent state.

17. A screening method for a compound having cytotoxic activity towards proliferating and/or quiescent cancer stem cells, comprising the steps of: (a) providing proliferating and/or quiescent cancer stem cells; (b) contacting the cells with a test compound; (c) determining the cytotoxicity of the test compound to the cells.

18. The method according to claim 17, wherein the step of determining the cytotoxicity comprises measuring the cell ATP-levels.

19. The method according to claim 17, wherein the step of determining the cytotoxicity comprises comparing the cell ATP-levels between test-compound-treated and untreated proliferating and/or quiescent cancer stem cells.
